(19) United States
(12) Patent Application Publication
(10) Pub. No.: US 2022/0255017 A1
(43) Pub. Date: Aug. 11, 2022

(54) ORGANIC ELECTROLUMINESCENT COMPOUND AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Hyo-Jung LEE, Gyeonggi-do (KR); Jin-Ri HONG, Gyeonggi-do (KR); Hyo-Soon PARK, Gyeonggi-do (KR); Sang-Hee CHO, Gyeonggi-do (KR); Dong-Hyung LEE, Gyeonggi-do (KR); Tae-Jun HAN, Gyeonggi-do (KR); Ye-Jin JEON, Gyeonggi-do (KR)

(21) Appl. No.: 17/612,155

(22) PCT Filed: Jun. 16, 2020
(86) PCT No.: PCT/KR2020/007766

§ 371 Date: Nov. 17, 2021

(30) Foreign Application Priority Data
Jun. 18, 2019 (KR) 10-2019-0072170
Jun. 11, 2020 (KR) 10-2020-0070845

(51) Int. Cl. H01L 51/00; C07D 401/04; C09K 11/06; C07D 401/10; C07D 209/94

(52) U.S. Cl. CPC H01L 51/0072; C07D 401/04; C09K 11/06; H01L 51/5072; H01L 51/0067; C07D 209/94; C07D 401/10

(57) ABSTRACT

The present disclosure relates to an organic electroluminescent compound and an organic electroluminescent device comprising the same. By comprising the organic electroluminescent compound according to the present disclosure, it is possible to provide an organic electroluminescent device having good thermal stability, low driving voltage, high luminous efficiency and/or improved lifetime properties.

### TECHNICAL FIELD

The present disclosure relates to an organic electroluminescent compound and an organic electroluminescent device comprising the same.

### BACKGROUND ART

An electroluminescent (EL) device is a self-light-emitting display device which has advantages in that it provides a wider viewing angle, a greater contrast ratio, and a faster response time. The first organic electroluminescent device was developed by Eastman Kodak in 1987, by using small aromatic diamine molecules and aluminum complexes as materials for forming a light-emitting layer (see Appl. Phys. Lett. 51, 913, 1987).

An organic electroluminescent device (OLED) changes electric energy into light by applying electricity to an organic electroluminescent material, and commonly comprises an anode, a cathode, and an organic layer formed between the two electrodes. The organic layer of the OLED may comprise a hole injection layer, a hole transport layer, a hole auxiliary layer, a light-emitting auxiliary layer, an electron blocking layer, a light-emitting layer, an electron buffer layer, a hole blocking layer, an electron transport layer, an electron injection layer, etc. The materials used in the organic layer can be classified into a hole injection material, a hole transport material, a hole auxiliary material, a light-emitting auxiliary material, an electron blocking material, a light-emitting material (including a host material and a dopant material), an electron buffer material, a hole blocking material, an electron transport material, an electron injection material, etc., depending on their functions. In the OLED, holes from the anode and electrons from the cathode are injected into a light-emitting layer by the application of electric voltage, and excitons having high energy are produced by the recombination of the holes and electrons. The organic light-emitting compound moves into an excited state by the energy and emits light from energy when the organic light-emitting compound returns to the ground state from the excited state.

The most important factor determining luminous efficiency in an OLED is the light-emitting materials. The light-emitting materials are required to have the following features: high quantum efficiency, high mobility of an electron and a hole, and uniformity and stability of the formed light-emitting material layer. The light-emitting material is classified into blue, green, or red light-emitting materials according to the light-emitting color, and further includes yellow or orange light-emitting materials. Furthermore, the light-emitting material may be classified into a host material and a dopant material in a functional aspect. Recently, an urgent task is the development of an OLED having high efficiency and long lifetime. In particular, the development of highly excellent light-emitting material over conventional light-emitting materials is urgently required, considering the EL properties necessary for medium- and large-sized OLED panels. For this, as a solvent in a solid state and an energy transmitter, a host material should preferably have high purity and a suitable molecular weight in order to be deposited under vacuum. Furthermore, a material is required to have high glass transition temperature and pyrolysis temperature to achieve thermal stability, high electrochemical stability to achieve a long lifetime, easy formability of an amorphous thin film, good adhesion with adjacent layers, and no movement between the layers.

In addition, the development of a material having an excellent thermal stability and capable of improving the performance of an organic electroluminescent device, such as driving voltage, luminous efficiency, and lifetime properties, in a hole transport layer, a buffer layer, an electron transport layer, etc., is required.

### DISCLOSURE OF INVENTION

### Technical Problem

The objective of the present disclosure is to provide an organic electroluminescent compound effective for producing an organic electroluminescent device having excellent thermal stability, low driving voltage, high luminous efficiency, and/or improved lifetime properties.

### Solution to Problem

A compound having a low glass transition temperature (Tg) may induce morphological changes even at low temperatures to reduce charge mobility in a thin film and degrade the performance of the OLED. As a result of intensive studies, the present inventors found that highly fused ring compounds according to present disclosure have a high glass transition temperature (Tg) despite a relatively low molecular weight, thereby resulting in low driving voltage, high luminous efficiency and/or improved lifetime properties, while providing good morphological stability.

Specifically, the present inventors found that the above objective can be achieved by an organic electroluminescent compound represented by the following formula 1:

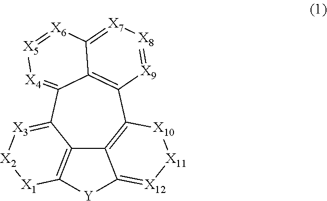

wherein

Y represents —N(R₁)—, —C(R₂)(R₃)—, —O—or —S—;

X₁ to X₁₂,each independently, represent N or CR₄;

R₁ represents —L—(Ar)ₐ;

L represents a single bond, a substituted or unsubstituted (C1-C30)alkylene, a substituted or unsubstituted (C6-C30)arylene, a substituted or unsubstituted (3- to 30-membered) heteroarylene, or a substituted or unsubstituted (C3-C30)cycloalkylene;

Ar, each independently, represents hydrogen, deuterium, a halogen, a cyano a substituted or unsubstituted (C1-C30)alkyl, a substituted or unsubstituted (C6-C30) aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30) alkyl(C6-C30)arylsilyl, a substituted or unsubstituted (C1-C30)alkyldi(C6-C30) arylsilyl, a substituted or unsubstituted tri(C6-C30)arylsilyl, a substituted or unsubstituted mono- or di- (C1-C30)alkylamino, a substituted or unsubstituted mono- or di- (C6-C30)arylamino, a substituted or unsubstituted (C1-C30)alkyl(C6-C30) arylamino, or a substituted or unsubstituted (C6-C30)aryl(3- to 30-membered) heteroarylamino;

R₂ to R₄, each independently, represent hydrogen, deuterium, a halogen, a cyano, a substituted or unsubstituted (C1-C30)alkyl, a substituted or unsubstituted (C6-C30) aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30) alkyl(C6-C30)arylsilyl, a substituted or unsubstituted (C1-C30)alkyldi(C6-C30) arylsilyl, a substituted or unsubstituted tri(C6-C30)arylsilyl, a substituted or unsubstituted mono- or di- (C1-C30)alkylamino, a substituted or unsubstituted mono- or di- (C6-C30)arylamino, or a substituted or unsubstituted (C1-C30)alkyl(C6-C30) arylamino; or R₂ and R₃ may be linked to each other to form a ring(s), or at least two of adjacent R4′s may be linked to each other to form a ring(s); R₂ and R₃ may be the same or different, and each of R₄ may be the same or different;

with the proviso that when both X₉ and X₁₀ represent CR₄, R₄′s in X₉ and X₁₀ are not fused with each other to form a pyrrole ring, a thiophene ring, or a furan ring;

the heteroaryl(ene) contains at least one heteroatom selected from B, N, O, S, Si, and P; and

a represents an integer of 1 or 2; where a is an integer of 2, each of Ar may be the same or different.

### Advantageous Effects of Invention

The organic electroluminescent compound according to the present disclosure can provide an organic electroluminescent device having low driving voltage, high luminous efficiency, and/or improved lifetime properties. In addition or alternatively, the organic electroluminescent compound according to the present disclosure has excellent thermal stability compared to other organic electroluminescent compounds having similar molecular weights.

### Mode for the invention

Hereinafter, the present disclosure will be described in detail, However, the following description is intended to explain the present disclosure, and is not meant in any way to restrict the scope of the present disclosure.

The term “organic electroluminescent compound” in the present disclosure means a compound that may be used in an organic electroluminescent device, and may be comprised in any layer constituting an organic electroluminescent device, as necessary.

The term “organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material, an electron buffer material, a hole blocking material, an electron transport material, or an electron injection material, etc.

The organic electroluminescent material of the present disclosure may comprise at least one compound represented by formula 1, The compound of formula 1 may be included in the light-emitting layer, but is not limited thereto. In this case, the compound of formula 1 may be included as a host. Also, the compound of formula 1 may be included in the electron transport zone. The compound of formula 1 may be included in the electron buffer layer, but is not limited thereto.

Herein, the term “(C1-C30)alkyl(ene)” is meant to be a linear or branched alkyl(ene) having 1 to 30 carbon atoms constituting the chain, in which the number of carbon atoms is preferably 1 to 10, and more preferably 1 to 6. The above alkyl may include methyl, ethyl, n-propyl, iso-propyl, n-butyl, iso-butyl, tert-butyl, etc. The term “(C3-C30) cycloalkyl” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, etc. The term “(3- to 7-membered)heterocycloalkyl” is meant to be a cycloalkyl having 3 to 7 ring backbone atoms, and including at least one heteroatom selected from the group consisting of B, N, O, S, Si, and P, and preferably the group consisting of O, S, and N. The above heterocycloalkyl may include tetrahydrofuran, pyrrolidine, thiolan, tetrahydropyran, etc, The term “(C6-C30) aryl(ene)” is meant to be a monocyclic or fused ring radical derived from an aromatic hydrocarbon having 6 to 30 ring backbone carbon atoms. The above aryl(ene) may be partially saturated, and may comprise a Spiro structure. The above aryl may include phenyl, biphenyl, terphenyl, naphthyl, binaphthyl, phenylnaphthyl, naphthylphenyl, fluorenyl, phenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, spirobifluorenyl, spiro[fluorene-benzofluoren]yl, etc. More specifically, the aryl may include phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzofluorenyl, dibenzofluorenyl, 2-biphenylyl, 3-biphenylyl, 4-biphenylyl, o-terphenyl, m-terphenyl-4-yl, m-terphenyl-3-yl, m-terphenyl-2-yl, p-terphenyl-4-yl, p-terphenyl -3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-tent-butylphenyl, p-(2-phenylpropyl)phenyl, 4″-methylbiphenylyl, 4″-tert-butyl-p-terphenyl-4-yl, 9,9-dimethyl -1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, etc.

The term “(3- to 30-membered)heteroaryl(ene)” is an aryl or an arylene having 3 to 30 ring backbone atoms, preferably 5 to 30 ring backbone atoms, and including at least one, preferably 1 to 4 heteroatoms selected from the group consisting of B, N, O, S, Si, and P, The above heteroaryl(ene) may be a monocyclic ring, or a fused ring condensed with at least one benzene ring; may be partially saturated; may be one formed by linking at least one heteroaryl or aryl group to a heteroaryl group via a single bond(s); and may comprise a Spiro structure, The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl such as benzofuranyl, benzothiophenyl, isobenzofuranyl, dibenzofuranyl, dibenzothiophenyl, benzonaphthothiophenyl, benzimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, quinoxalinyl, benzoquinoxalinyl, carbazolyl, benzocarbazolyl, dibenzocarbazolyl, phenoxazinyl, phenanthridinyl, benzodioxolyl, naphthyridinyl, benzofuropyrimidinyl, benzothienopyrimidinyl, indolopyrimidinyl, indenopyrimidinyl, etc. More specifically, the heteroaryl may include 1-pyrrolyl, 2-pyrrolyl, 3-pyrrolyl, pyrazinyl, 2-pyridinyl, 2-pyrimidinyl, 4-pyrimidinyl, 5-pyrimidinyl, 6-pyrimidinyl, 1,2,3-triazin-4-yl, 1,2,4-triazin-3-yl, 1 ,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridinyl, 3-imidazopyridinyl, 5-imidazopyridinyl, 6-imidazopyridinyl, 7-imidazopyridinyl, 8-imidazopyridinyl, 3-pyridinyl, 4-pyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-fu 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazolyl-1-yl, azacarbazolyl-2-yl, azacarbazolyl-3-yl, azacarbazolyl-4-yl, azacarbazolyl-5-yl, azacarbazolyl-6-yl, azacarbazolyl-7-yl, azacarbazolyl-8-yl, azacarbazolyl-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol -1-yl, 2-methylpyrrol-3-yl, 2-methylpyrrol-4-yl, 2-methylpyrrol-5-yl, 3-methylpyrrol -1-yl, 3-methylpyrrol-2-yl, 3-methylpyrrol-4-yl, 3-methylpyrrol-5-yl, 2-tert-butylpyrrol -4-yl, 3-(2-phenylpropyl)pyrrol-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert -butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, etc. “Halogen” includes F, Cl, Br, and I.

In addition, “ortho (o-),” “meta (m-),” and “para (p-)” are prefixes, which represent the relative positions of substituents, respectively. Ortho indicates that two substituents are adjacent to each other, and for example, when two substituents in a benzene derivative occupy positions 1 and 2, it is called an ortho position. Meta indicates that two substituents are at positions 1 and 3, and for example, when two substituents in a benzene derivative occupy positions 1 and 3, it is called a meta position. Para indicates that two substituents are at positions 1 and 4, and for example, when two substituents in a benzene derivative occupy positions 1 and 4, it is called a pars position.

Herein, “substituted” in the expression “substituted or unsubstituted” means that a hydrogen atom in a certain functional group is replaced with another atom or another functional group, i.e., a substituent. In the present disclosure, the substituents of the substituted alkyl(ene), the substituted aryl(ene), the substituted heteroaryl(ene), the substituted cycloalkyl(ene), the substituted alkoxy, the substituted trialkylsilyl, the substituted dialkylarylsilyl, the substituted alkyldiarylsilyl, the substituted triarylsilyl, the substituted mono- or di- alkylamino, the substituted mono- or di- arylamino, the substituted alkylarylamino, and the substituted arylheteroarylamino, each independently, are at least one selected from the group consisting of deuterium; a halogen; a cyano; a carboxyl; a nitro; a hydroxyl; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30) cycloalkyl; a (C3-C30)cycloalkenyl; a (3- to 7-membered)heterocycloalkyl; a (C6-C30) aryloxyl, a (C6-C30)arylthio; a (3- to 30-membered)heteroaryl unsubstituted or substituted with a (C6-C30)aryl(s); a (C6-C30)aryl unsubstituted or substituted with at least one of a (C1-C30)alkyl(s) and a (3- to 30-membered)heteroaryl(s); a tri(C1-C30) alkylsilyl; a tri(C6-C30)arylsilyl; a di(C1-C30)alkyl(C6-C30)arylsilyl; a (C1-C30) alkyldi(C6-C30)arylsilyl; an amino; a mono- or di- (C1-C30)alkylamino; a mono- or di- (C6-C30)arylamino unsubstituted or substituted with a (C1-C30)alkyl(s); a (C1-C30) alkyl(C6-C30)alyamino; a (C6-C30)aryl(3- to 30-membered)heteroarylamino unsubstituted or substituted with a (C6-C30)aryl(s); a (C1-C30)alkylcarbonyl; a (C1-C30) alkoxycarbonyl; a (C6-C30)arylcarbonyl; a di(C6-C30)arylboronyl; a di(C1-C30) alkylboronyl; a (C1-C30)alkyl(C6-C30)arylboronyl, a (C6-C30)aryl(C1-C30)alkyl; and a (C1-C30)alkyl(C6-C30)aryl. According to one embodiment of the present disclosure; the substituents, each independently, are at least one selected from the group consisting of deuterium; a (C1-C20)alkyl, a (5- to 25-membered)heteroaryl unsubstituted or substituted with a (C6-C25)aryl(s); a (C6-C25)aryl unsubstituted or substituted with at least one of a (C1-C20)alkyl(s) and a (3- to 20-membered) heteroaryl(s); an amino; a mono- or di- (C6-C25)arylamino unsubstituted or substituted with a (C1-C10)alkyl(s); and a (C6-C18)aryl(3- to 25-membered) heteroarylamino unsubstituted or substituted with a (C6-C18)aryl(s). According to another embodiment of the present disclosure, the substituents, each independently, are at least one selected from the group consisting of a (C1-C10)alkyl, a (5- to 20-membered)heteroaryl unsubstituted or substituted with a (C6-C18)aryl(s); a (C6-C18)aryl unsubstituted or substituted with at least one of a (C1-C10)alkyl(s) and a (5- to 20-membered)heteroaryl(s); a di(C6-C18)arylamino substituted with a (C1-C10) alkyl(s); and a (C6-C18)aryl(3- to 20-membered)heteroarylamino substituted with a (C6-C18)aryl(s). For example, the substituents, each independently, may be at least one selected from the group consisting of a methyl, a phenyl unsubstituted or substituted with a diphenyltriazinyl(s), a naphthyl, a biphenyl, a dimethylfluorenyl, a triazinyl substituted with a phenyl(s) and/or a biphenyl(s), a quinazolinyl substituted with a phenyl(s), a quinoxalinyl substituted with a phenyl(s), a dibenzofuranyl, a dimethyffluorenylbiphenylamino, a dimethylfluorenylphenylamino, a phenylcarbazolylbiphenylamino, and a phenylcarbazolylphenylamino.

In the formulas of the present disclosure, a ring formed by a linkage of adjacent substituents means that at least two adjacent substituents are linked to or fused with each other to form a substituted or unsubstituted mono- or polycyclic (3- to 30-membered) alicyclic or aromatic ring, or the combination thereof; preferably, a substituted or unsubstituted mono- or polycyclic (3- to 26-membered) alicyclic or aromatic ring, or the combination thereof; and more preferably, an unsubstituted mono- or polycyclic (5- to 10-membered) aromatic ring. For example, the ring may be a benzene ring, an indene ring, an indole ring, a benzofuran ring, or a benzothiophene ring, etc, In addition, the ring may contain at least one heteroatom selected from B, N, O, S, Si, and P, preferably at least one heteroatom selected from N, O,and S.

Herein, the heteroaryl, the heteroarylene and the heterocycloalkyl, each independently, may contain at least one heteroatom selected from B, N, O, S, Si, and P. In addition, the heteroatom may be bonded to at least one selected from the group consisting of hydrogen, deuterium, a halogen, a cyano, a substituted or unsubstituted (C1-C30)alkyl, a substituted or unsubstituted (C6-C30)aryl, a substituted or unsubstituted (5- to 30-membered)heteroaryl, a substituted or unsubstituted (C3-C30) cycloalkyl, a substituted or unsubstituted (C1-C30)alkoxy, a substituted or unsubstituted tri(C1-C30)alkylsilyl, a substituted or unsubstituted di(C1-C30)alkyl(C6-C30) arylsilyl, a substituted or unsubstituted (C1-C30)alkyldi(C6-C30)arylsilyl, a substituted or unsubstituted tri(C6-C30)arylsilyl, a substituted or unsubstituted mono- or di- (C1-C30)alkylamino, a substituted or unsubstituted mono- or di- (C6-C30) arylamino, and a substituted or unsubstituted (C1-C30)alkyl(C6-C30)arylamino.

Hereinafter, the organic electroluminescent compound represented by formula 1 will be described in more detail.

In formula 1, Y represents —N(R₁)—, —C(R₂)(R₃)—, —O—or —S—; wherein R₁ represents —L—(Ar)ₐ.

In formula 1, X₁ to X₁₂, each independently, represent N or CR₄. According to one embodiment of the present disclosure, X₁ to X₁₂, each independently, represent CR₄. According to another embodiment of the present disclosure, any one of X₁ to X₁₂ may represent N.

L represents a single bond, a substituted or unsubstituted (C1-C30)alkylene, a substituted or unsubstituted (C6-C30)arylene, a substituted or unsubstituted (3- to 30-membered) heteroarylene, or a substituted or unsubstituted (C3-C30)cycloalkylene. According to one embodiment of the present disclosure, L represents a single bond, a substituted or unsubstituted C(6-C25)arylene, or a substituted or unsubstituted (5- to 25-membered)heteroarylene. According to another embodiment of the present disclosure, L represents a single bond, an unsubstituted (C6-C18)arylene, or a (5- to 20-membered)heteroarylene unsubstituted or substituted with a (C1-C30)alkyl(s) and/or a (C6-C30)aryl(s). For example, L may represent a single bond, a phenylene, a naphthylene, a biphenylene, an anthracenylene, a pyridylene, a pyrimidinylene, a triazinylene, a quinoxalinylene, a quinazolinylene, a benzoquinoxalinylene, a benzofuropyrimidinylene, a benzothienopyrimidinylene, an indolopyrimidinylene substituted with a phenyl(s), or an indenopyrimidinylene substituted with a methyl(s).

Ar, each independently, represents hydrogen, deuterium, a halogen, a cyano, a substituted or unsubstituted (C1-C30)alkyl, a substituted or unsubstituted (C6-C30) aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30) alkyl(C6-C30)arylsilyl, a substituted or unsubstituted (C1-C30)alkyldi(C6-C30) arylsilyl, a substituted or unsubstituted tri(C6-C30)arylsilyl, a substituted or unsubstituted mono- or di- (C1-C30)alkylamino, a substituted or unsubstituted mono- or di- (C6-C30)arylamino, a substituted or unsubstituted (C1-C30)alkyl(C6-C30) arylamino, or a substituted or unsubstituted (C6-C30)aryl(3- to 30-membered) heteroarylamino. According to one embodiment of the present disclosure, Ar, each independently, represents a substituted or unsubstituted (C6-C25)aryl, a substituted or unsubstituted (5- to 25-membered)heteroaryl, a substituted or unsubstituted mono- or di- (C6-C25)arylamino, or a substituted or unsubstituted (C6-C25) aryl(5- to 25-membered)heteroarylamino. According to another embodiment of the present disclosure, Ar, each independently, represents a (C6-C20)aryl unsubstituted or substituted with a (C6-C18)aryl(s); a (5- to 25-membered)heteroaryl unsubstituted or substituted with at least one of a (C1-C10)alkyl(s), a (C6-C18)aryl(s) and a (5- to 25-membered)heteroaryl(s); a di(C6-C18)arylamino unsubstituted or substituted with a (C1-C10)alkyl(s); or a (C6-C18)aryl(5- to 20-membered) heteroarylamino unsubstituted or substituted with a (C6-C18)aryl(s). Specifically, Ar may represent a substituted or unsubstituted pheny a substituted or unsubstituted naphthyl, a substituted or unsubstituted biphenyl, a substituted or unsubstituted terphenyl, a substituted or unsubstituted fluorenyl, a substituted or unsubstituted anthracenyl, a substituted or unsubstituted fluoranthenyl, a substituted or unsubstituted triazinyl, a substituted or unsubstituted pyridyl, a substituted or unsubstituted pyrimidinyl, a substituted or unsubstituted benzofuropyrimidinyl, a substituted or unsubstituted indolopyrimidinyl, a substituted or unsubstituted indenopyrimidinyl, a substituted or unsubstituted benzothienopyrimidinyl, a substituted or unsubstituted acenaphthopyrimidinyl, a substituted or unsubstituted pyrimidoindole, a substituted or unsubstituted quinazolinyl, a substituted or unsubstituted benzoquinazolinyl, a substituted or unsubstituted quinoxalinyl, a substituted or unsubstituted benzoquinoxalinyl, a substituted or unsubstituted dibenzoquinoxalinyl, a substituted or unsubstituted quinolyl, a substituted or unsubstituted benzoquinolyl, a substituted or unsubstituted isoquinolyl, a substituted or unsubstituted benzoisoquinolyl, a substituted or unsubstituted benzothienoquinolyl, a substituted or unsubstituted benzofuroquinolyl, a substituted or unsubstituted triazolyl, a substituted or unsubstituted pyrazolyl, a substituted or unsubstituted carbazolyl, a substituted or unsubstituted dibenzothiophenyl, a substituted or unsubstituted benzothiophenyl, a substituted or unsubstituted dibenzofuranyl, a substituted or unsubstituted benzofuranyl, a substituted or unsubstituted naphthyridinyl, a substituted or unsubstituted benzothiazolinyl, a substituted or unsubstituted phenanthroimidazolyl, a substituted or unsubstituted diphenylamino, a substituted or unsubstituted phenylbiphenylamino, a substituted or unsubstituted fluorenylphenylamino, a substituted or unsubstituted fluorenylbiphenylamino, a substituted or unsubstituted carbazolylphenylamino, a substituted or unsubstituted dibenzothiophenylphenylamino, a substituted or unsubstituted dibenzofuranylphenylamino, a substituted or unsubstituted benzofuropyrazinyl, a substituted or unsubstituted benzofuroquinoxalinyl, a substituted or unsubstituted benzothienoquinoxalinyl, a substituted or unsubstituted benzothienoquinazolinyl, a substituted or unsubstituted benzofuroquinazolinyl, a substituted or unsubstituted benzothienopyrazinyl, a substituted or unsubstituted naphthofuropyrazinyl, a substituted or unsubstituted naphthothienopyrazinyl, a substituted or unsubstituted naphthofuropyrimidinyl, a substituted or unsubstituted naphthothienopyrimidinyl, a substituted or unsubstituted spiro[fluorene-indenopyrimidin]yl, a substituted or unsubstituted spiro[fluorene-indenopyrazin]yl, a substituted or unsubstituted acenaphthopyrazinyl, a substituted or unsubstituted benzimidazotriazinyl, a substituted or unsubstituted pyridoindazolyl, a substituted or unsubstituted dibenzoquinazolinyl, a substituted or unsubstituted chromenoquinazolinyl, a substituted or unsubstituted thiochromenoquinazolinyl, or a substituted or unsubstituted dimethyl benzoperimidinyl. For example, Ar, each independently, may represent an unsubstituted phenyl, an unsubstituted naphthyl, an unsubstituted biphenyl, an unsubstituted terphenyl, a substituted anthracenyl, a substituted pyrimidinyl, a substituted triazinyl, a substituted quinoxalinyl, a substituted quinazolinyl, a substituted naphthyridinyl, a substituted benzoquinoxalinyl, an unsubstituted dibenzofuranyl, a substituted benzofuropyrimidinyl, a substituted benzothienopyrimidinyl, a substituted acenaphthopyrimidinyl, a substituted indolopyrimidinyl, a substituted indenopyrimidinyl, an unsubstituted diphenylamino, an unsubstituted phenylbiphenylamino, a substituted fluorenylphenylamino, a substituted fluorenylbiphenylamino, a substituted carbazolylphenylamino, a substituted benzofuropyrazinyl, a substituted benzofuroquinoxalinyl, a substituted benzothienoquinoxalinyl, a substituted benzothienoquinazolinyl, a substituted benzofuroquinazolinyl, a substituted benzothienopyrazinyl, a substituted naphthofuropyrazinyl, a substituted or unsubstituted naphthothienopyrazinyl, a substituted or unsubstituted naphthofuropyrimidinyl, a substituted or unsubstituted naphthothienopyrimidinyl, a substituted spiro[fluorene-indenopyrimidin]yl, a substituted spiro[fluorene-indenopyrazin]yl, a substituted or unsubstituted acenaphthopyrazinyl, an unsubstituted benzimidazotriazinyl, an unsubstituted pyridoindazolyl, a substituted dibenzoquinazolinyl, an unsubstituted chromenoquinazolinyl, an unsubstituted thiochromenoquinazolinyl, or an unsubstituted dimethyl benzoperimidinyl. The substituent of the substituted anthracenyl, the substituted pyrimidinyl, the substituted triazinyl, the substituted quinoxalinyl, the substituted quinazo inyl, the substituted naphthyridinyl, the substituted benzoquinoxalinyl, the substituted benzofuropyrimidinyl, the substituted benzothienopyrimidinyl, the substituted acenaphthopyrimidinyl, the substituted indolopyrimidinyl, the substituted indenopyrimidinyl, the substituted fluorenylphenylamino, the substituted fluorenylbiphenylamino, the substituted carbazolylphenylamino, the substituted benzofuropyrazinyl, the substituted benzofuroquinoxalinyl, the substituted benzothienoquinoxalinyl, the substituted benzothienoquinazolinyl, the substituted benzofuroquinazolinyl, the substituted benzothienopyrazinyl, the substituted naphthofuropyrazinyl, the substituted naphthothienopyrazinyl, the substituted naphthofuropyrimidinyl, the substituted naphthothienopyrimidinyl, the substituted spiro[fluorene-indenopyrimidin]yl, the substituted spiro[fluorene-indenopyrazin]yl, the substituted acenaphthopyrazinyl, and the substituted dibenzoguinazolinyl, each independently, may be at least one selected from the group consisting of a methyl, a phenyl, a naphthyl, a biphenyl, and a dibenzofuranyl.

In formula 1, R₂ to R₄, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered) heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30) arylsilyl, a substituted or unsubstituted mono- or di- (C1-C30)alkylamino, a substituted or unsubstituted mono- or di- (C6-C30)arylamino, or a substituted or unsubstituted (C1-C30)alkyl(C6-C30)arylamino; or R₂ and R₃ may be linked to each other to form a ring(s), or at least two of adjacent R4′s may be linked to each other to form a ring(s). When both X₉ and X₁₀ represent CR₄, R4′s in X₉ and X₁₀ are not fused with each other to form a pyrrole ring, a thiophene ring, or a furan ring. R₂ and R₃ may be the same or different, and each of R₄ may be the same or different.

According to one embodiment of the present disclosure, R₂ and R₃, each independently, represent a substituted or unsubstituted (C1-C20)alkyl, a substituted or unsubstituted (C6-C25)aryl, or a substituted or unsubstituted (5- to 25-membered) heteroaryl; or R₂ and R₃ may be linked to each other to form a spiro ring. R₂ and R₃ may be the same or different. According to another embodiment of the present disclosure, R₂ and R₃, each independently, represent an unsubstituted (C1-C10) alkyl. For example, R₂ and R₃ may be a methyl.

According to one embodiment of the present disclosure, R₄, each independently, represents hydrogen, deuterium, a substituted or unsubstituted (C6-C25)aryl, or a substituted or unsubstituted (5- to 25-membered)heteroaryl; or at least two of adjacent R₄'s may be linked to each other to form a ring(s). According to another embodiment of the present disclosure, R4, each independently, represents hydrogen; a (C6-C20) aryl unsubstituted or substituted with a (C6-C18)aryl(s), a (3- to 30-membered) heteroaryl(s), a di(C6-C18)arylamino(s), or a (C6-C18)aryl(5- to 25-membered) heteroaryl(s); or a (5- to 20-membered)heteroaryl unsubstituted or substituted with a (C6-C18)aryl(s); or two adjacent R₄'s may be linked to each other to form a substituted or unsubstituted mono- or polycyclic (3- to 30-membered) alicyclic or aromatic ring, or the combination thereof. The ring may contain at least one heteroatom selected from B, N, O, S, Si, and P. For example, R₄, each independently, represents hydrogen, a substituted or unsubstituted phenyl, a substituted naphthyl, a anthracenyl substituted with a phenyl(s), a triazinyl substituted with a phenyl(s), a quinoxalinyl substituted with a phenyl(s), or a quinazolinyl substituted with a phenyl(s); or two adjacent R₄'s may be linked to each other to form an unsubstituted benzene ring, an indene ring substituted with a methyl(s), a substituted indole ring, an unsubstituted benzofuran ring, or an unsubstituted benzothiophene ring. The substituent of the substituted phenyl and the substituted naphthyl, each independently, may be at least one selected from the group consisting of a triazinyl substituted with a phenyl(s), a dimethylfluorenylbiphenylamino, a dimethylfluorenylphenylamino, a phenylcarbazolylbiphenylamino, and a phenylcarbazolylphenylamino, The substituent of the substituted indole ring may be at least one selected from the group consisting of a phenyl unsubstituted or substituted with a diphenyltriazinyl(s); a triazinyl substituted with a phenyl(s) and/or a biphenyl(s); a quinazolinyl substituted with a phenyl(s); a quinoxalinyl substituted with a phenyl(s); a naphthyl; and a dimethylfluorenyl.

In formula 1, a represents an integer of 1 or 2; where a is an integer of 2, each of Ar may be the same or different.

According to one embodiment of the present disclosure, two of adjacent X₁ to X₁₂ in formula 1 are CR₄, two adjacent R₄'s may be fused in the form of any one of the following formulas 2 to 6 to form a ring; and the ring may present one or more in one compound represented by formula 1.

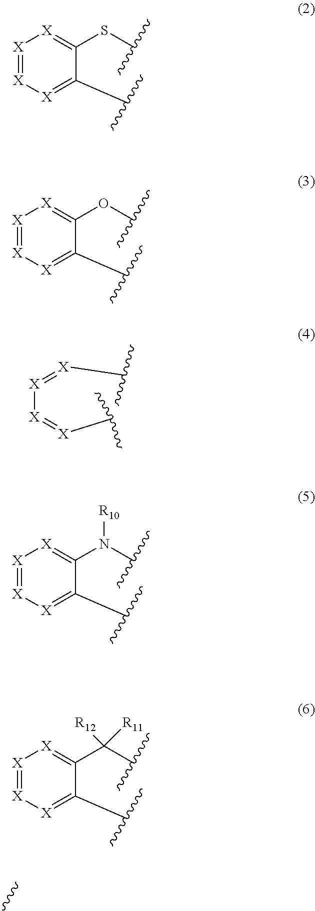

In formulas 2 to 6, X, each independently, represents N or CH. According to one embodiment of the present disclosure, in any one of formulas 2 to 6, all of X may represent CH, or any one of X may represent N.

In formula 5, R₁₀ represents hydrogen, deuterium, a halogen, a cyano, a substituted or unsubstituted (C1-C30)alkyl, a substituted or unsubstituted (C6-C30) aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30) alkyl(C6-C30)arylsilyl, a substituted or unsubstituted (C1-C30)alkyldi(C6-C30) arylsilyl, a substituted or unsubstituted tri(C6-C30)arylsilyl, a substituted or unsubstituted mono- or di- (C1-C30)alkylamino, a substituted or unsubstituted mono-or di- (C6-C30)arylamino, or a substituted or unsubstituted (C1-C30)alkyl(C6-C30) arylamino. According to one embodiment of the present disclosure, R₁₀ represents a substituted or unsubstituted (C6-C25)aryl, or a substituted or unsubstituted (5- to 25-membered)heteroaryl. According to another embodiment of the present disclosure, Rio represents a (C6-C18)aryl unsubstituted or substituted with a (C1-C10)alkyl(s) and/or (5- to 20-membered)heteroaryl(s); or a (5- to 25-membered) heteroaryl unsubstituted or substituted with a (C6-C18)aryl(s). For example, R₁₀ may be a phenyl; a phenyl substituted with a diphenyltriazinyl(s); a naphthyl; a dimethylfluorenyl; a pyridyl; a triazinyl substituted with a phenyl(s) and/or a biphenyl(s); a quinazolinyl substituted with a phenyl(s); or a quinoxalinyl substituted with a phenyl(s).

In formula 6, R₁₁ and R12, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered) heteroaryl, or a substituted or unsubstituted (C3-C30)cycloalkyl; or R31 and R₁₂ may be linked to each other to form a ring(s). According to one embodiment of the present disclosure, R₁₁ and R₁₂, each independently, represent a substituted or unsubstituted (C1-C20)alkyl, a substituted or unsubstituted (C6-C25)aryl, or a substituted or unsubstituted (5- to 25-membered)heteroaryl. R₁₁ and R₁₂ may be the same or different. According to another embodiment of the present disclosure, R₁₁ and R₁₂, each independently, represent an unsubstituted Malkyl. For example, R₁₁ and R₁₂ may be a methyl.

The compound represented by formula 1 may be specifically exemplified by the following compounds, but is not limited thereto.

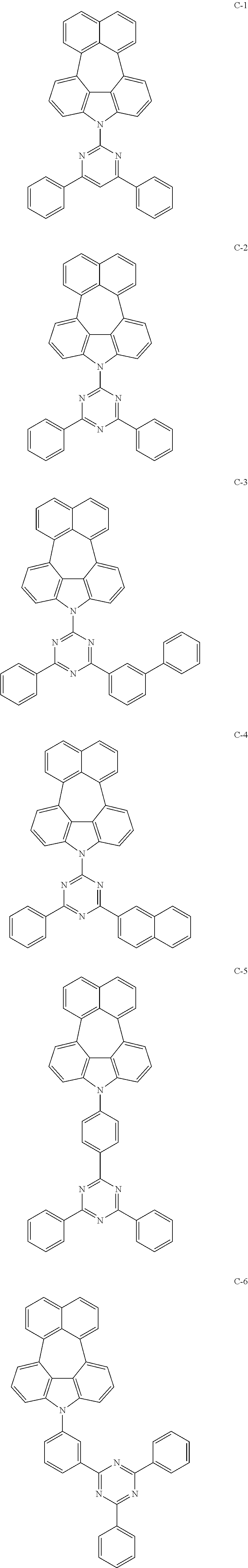 [SMILES: c1ccc(-c2cc(-c3ccccc3)nc(-n3c4cccc5c4c4c(cccc43)-c3cccc4cccc-5c34)n2)cc1 ; c1ccc(-c2nc(-c3ccccc3)nc(-n3c4cccc5c4c4c(cccc43)-c3cccc4cccc-5c34)n2)cc1 ; c1ccc(-c2cccc(-c3nc(-c4ccccc4)nc(-n4c5cccc6c5c5c(cccc54)-c4cccc5cccc-6c45)n3)c2)cc1 ; c1ccc(-c2nc(-c3ccc4ccccc4c3)nc(-n3c4cccc5c4c4c(cccc43)-c3cccc4cccc-5c34)n2)cc1 ; c1ccc(-c2nc(-c3ccccc3)nc(-c3ccc(-n4c5cccc6c5c5c(cccc54)-c4cccc5cccc-6c45)cc3)n2)cc1 ; c1ccc(-c2nc(-c3ccccc3)nc(-c3cccc(-n4c5cccc6c5c5c(cccc54)-c4cccc5cccc-6c45)c3)n2)cc1]

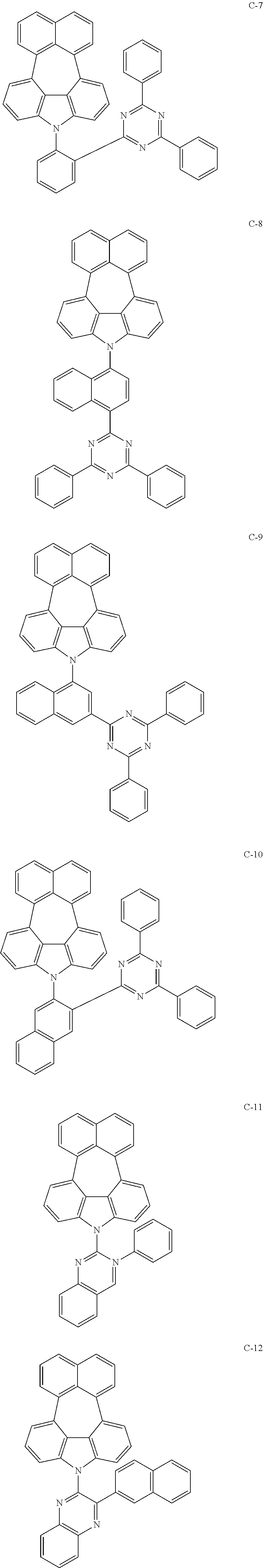

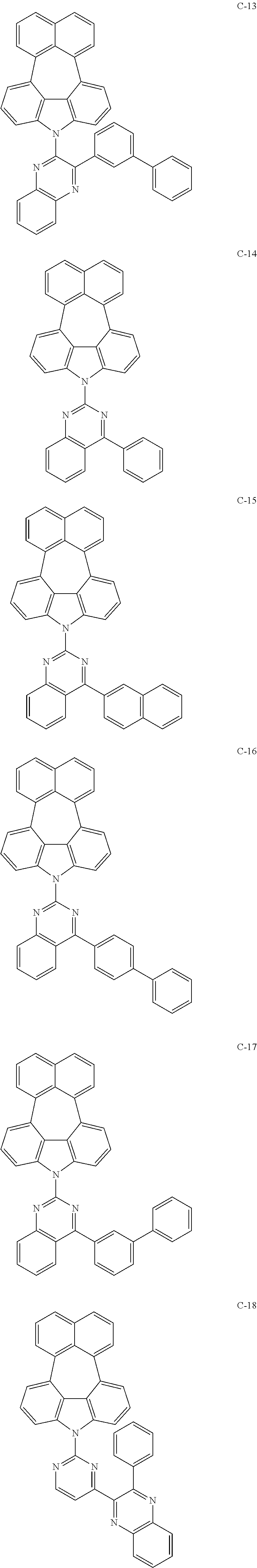

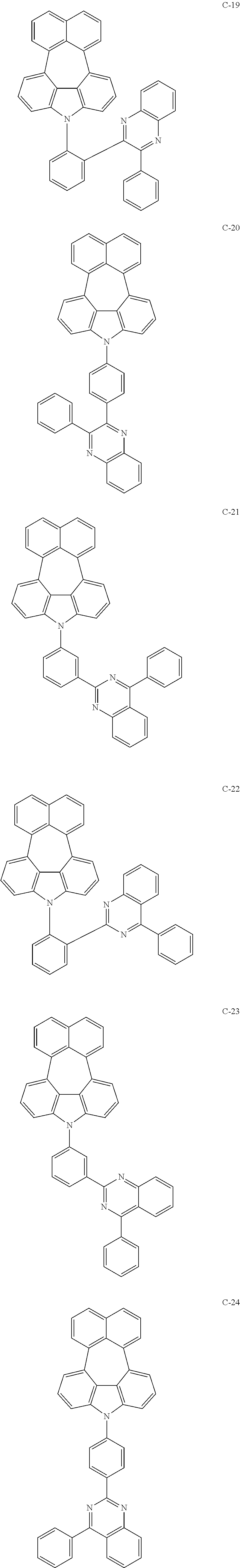 [SMILES: c1ccc(-c2nc3ccccc3nc2-c2ccccc2-n2c3cccc4c3c3c(cccc32)-c2cccc3cccc-4c23)cc1 ; c1ccc(-c2nc3ccccc3nc2-c2ccc(-n3c4cccc5c4c4c(cccc43)-c3cccc4cccc-5c34)cc2)cc1 ; c1ccc(-c2nc(-c3cccc(-n4c5cccc6c5c5c(cccc54)-c4cccc5cccc-6c45)c3)nc3ccccc23)cc1 ; c1ccc(-c2nc(-c3ccccc3-n3c4cccc5c4c4c(cccc43)-c3cccc4cccc-5c34)nc3ccccc23)cc1 ; c1ccc(-c2nc(-c3cccc(-n4c5cccc6c5c5c(cccc54)-c4cccc5cccc-6c45)c3)nc3ccccc23)cc1 ; c1ccc(-c2nc(-c3ccc(-n4c5cccc6c5c5c(cccc54)-c4cccc5cccc-6c45)cc3)nc3ccccc23)cc1]

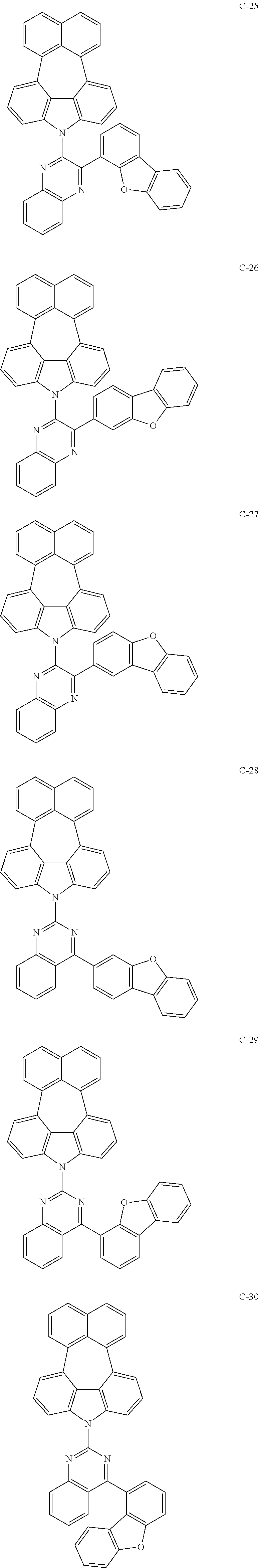

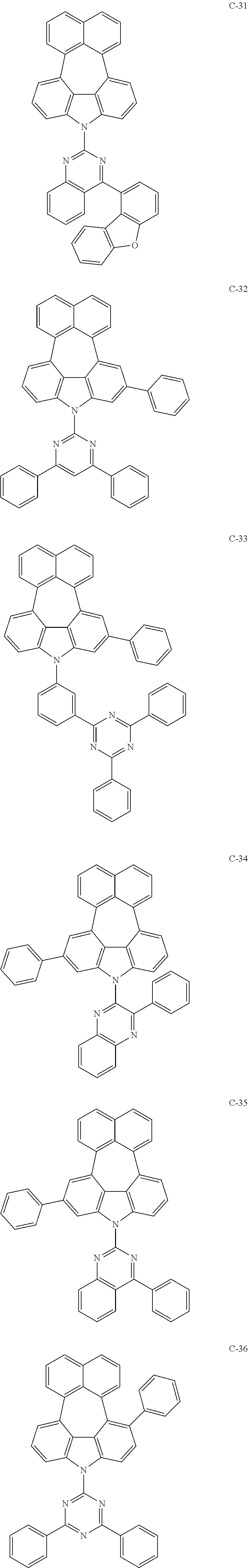

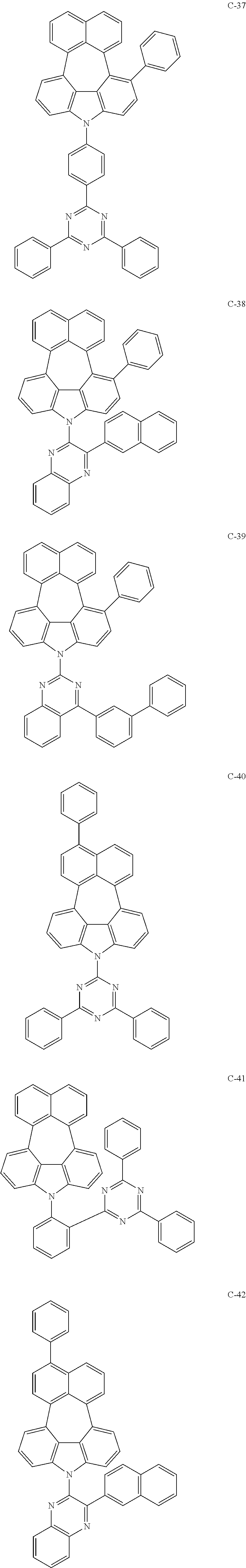

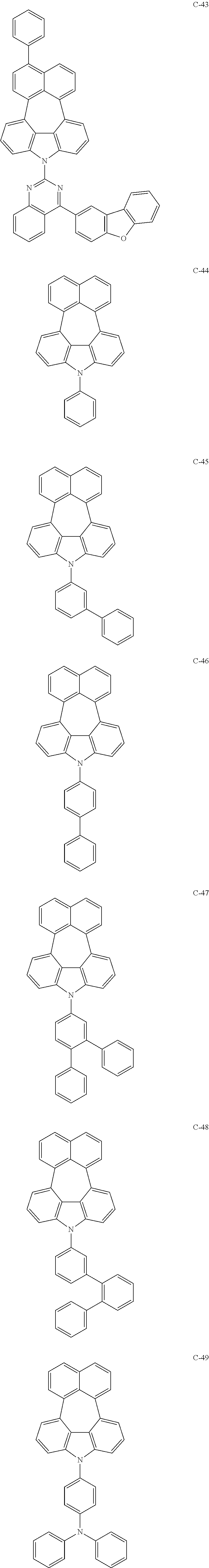

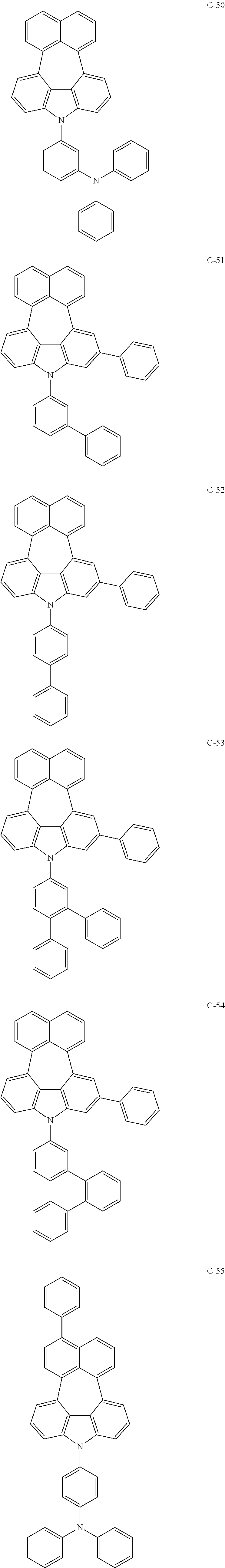 [SMILES: c1ccc(N(c2ccccc2)c2cccc(-n3c4cccc5c4c4c(cccc43)-c3cccc4cccc-5c34)c2)cc1 ; c1ccc(-c2cccc(-n3c4cccc5c4c4c(cc(-c6ccccc6)cc43)-c3cccc4cccc-5c34)c2)cc1 ; c1ccc(-c2ccc(-n3c4cccc5c4c4c(cc(-c6ccccc6)cc43)-c3cccc4cccc-5c34)cc2)cc1 ; c1ccc(-c2cc3c4c5c(cccc5n(-c5ccc(-c6ccccc6)c(-c6ccccc6)c5)c4c2)-c2cccc4cccc-3c24)cc1 ; c1ccc(-c2cc3c4c5c(cccc5n(-c5cccc(-c6ccccc6-c6ccccc6)c5)c4c2)-c2cccc4cccc-3c24)cc1 ; c1ccc(-c2ccc3c4c(cccc24)-c2cccc4c2c2c-3cccc2n4-c2ccc(N(c3ccccc3)c3ccccc3)cc2)cc1]

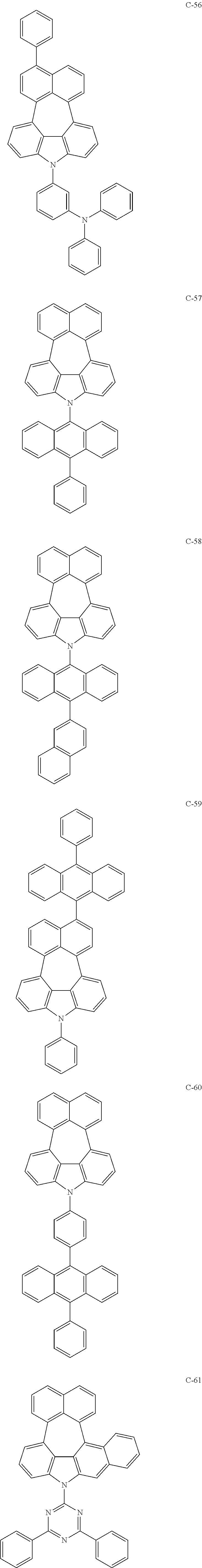

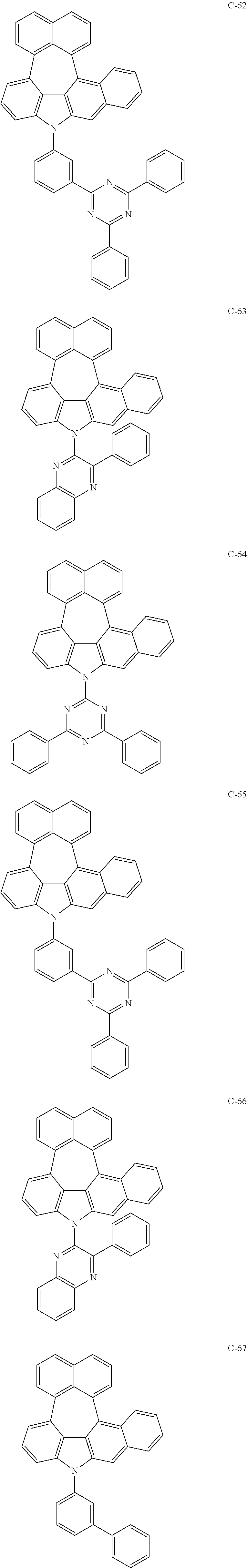

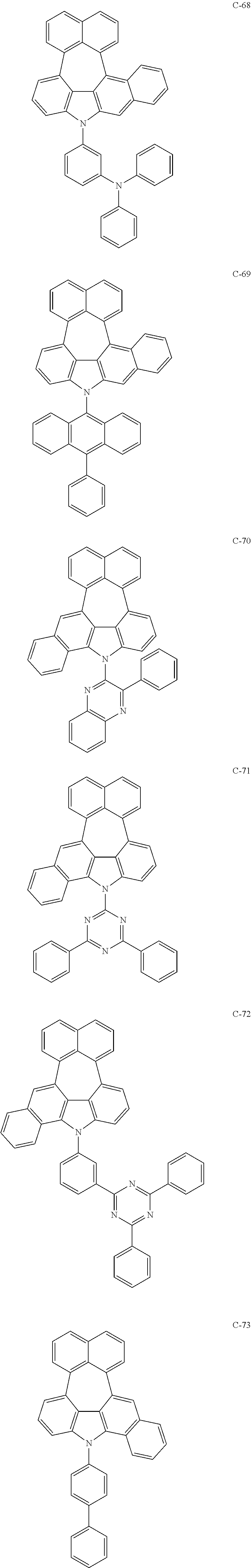

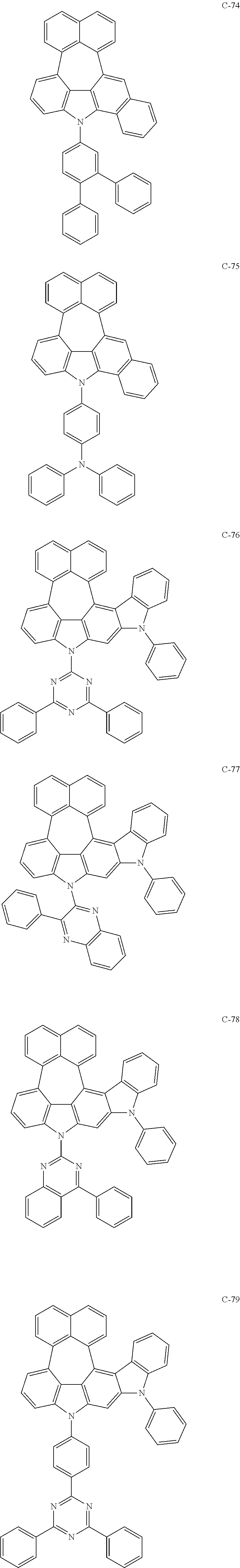

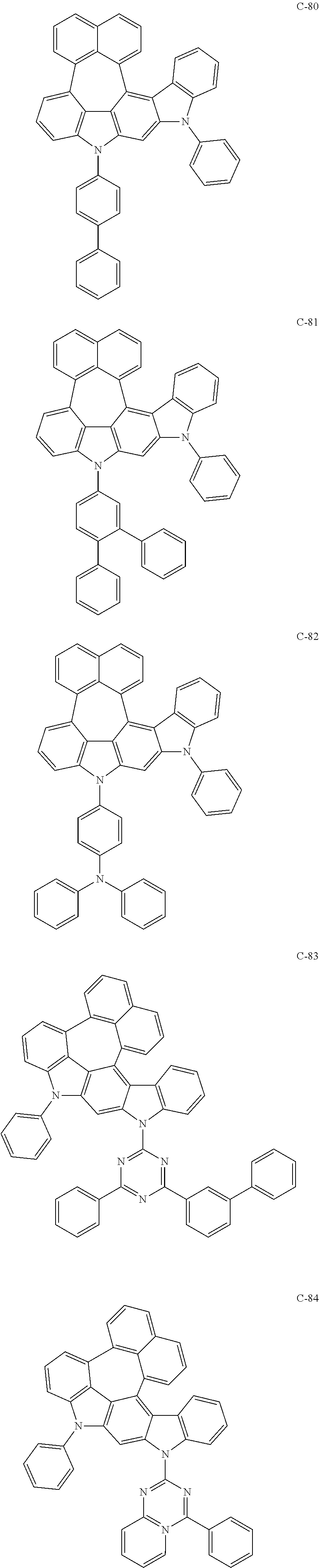

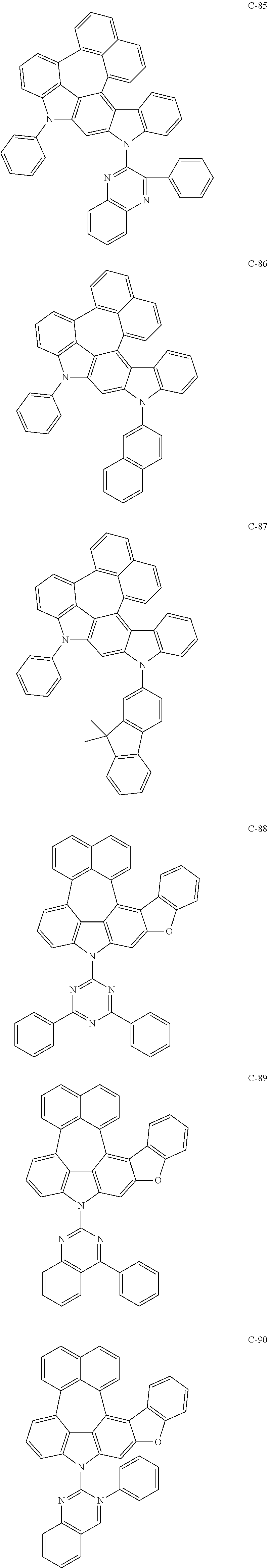

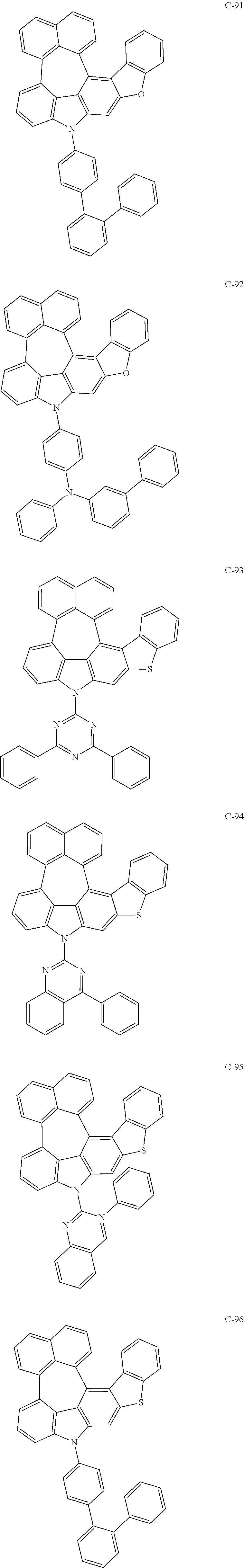

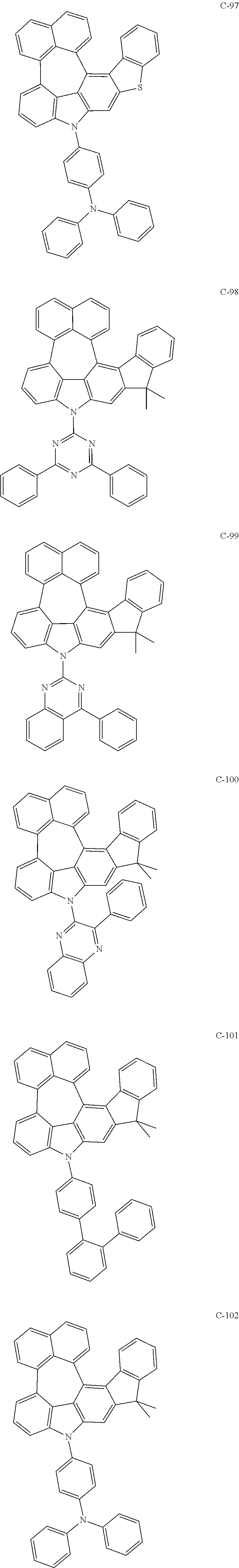

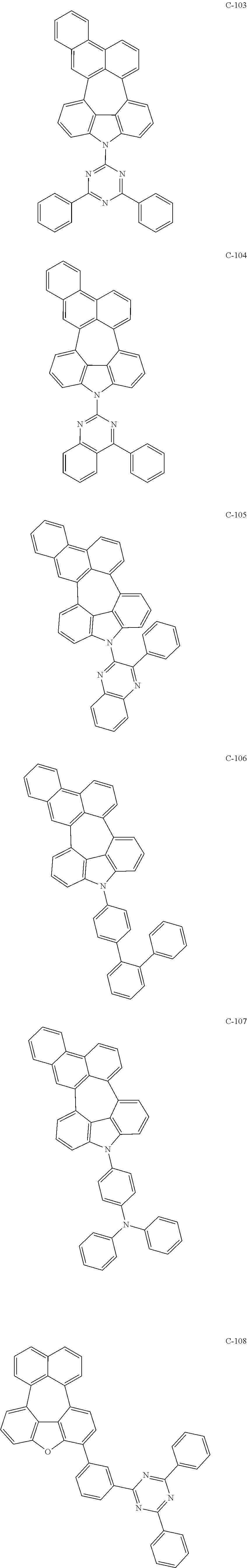 [SMILES: c1ccc(-c2nc(-c3ccccc3)nc(-n3c4cccc5c4c4c(cccc43)-c3cc4ccccc4c4cccc-5c34)n2)cc1 ; c1ccc(-c2nc(-n3c4cccc5c4c4c(cccc43)-c3cc4ccccc4c4cccc-5c34)nc3ccccc23)cc1 ; c1ccc(-c2nc3ccccc3nc2-n2c3cccc4c3c3c(cccc32)-c2cc3ccccc3c3cccc-4c23)cc1 ; c1ccc(-c2ccccc2-c2ccc(-n3c4cccc5c4c4c(cccc43)-c3cc4ccccc4c4cccc-5c34)cc2)cc1 ; c1ccc(N(c2ccccc2)c2ccc(-n3c4cccc5c4c4c(cccc43)-c3cc4ccccc4c4cccc-5c34)cc2)cc1 ; c1ccc(-c2nc(-c3ccccc3)nc(-c3cccc(-c4ccc5c6c4oc4cccc(c46)-c4cccc6cccc-5c46)c3)n2)cc1]

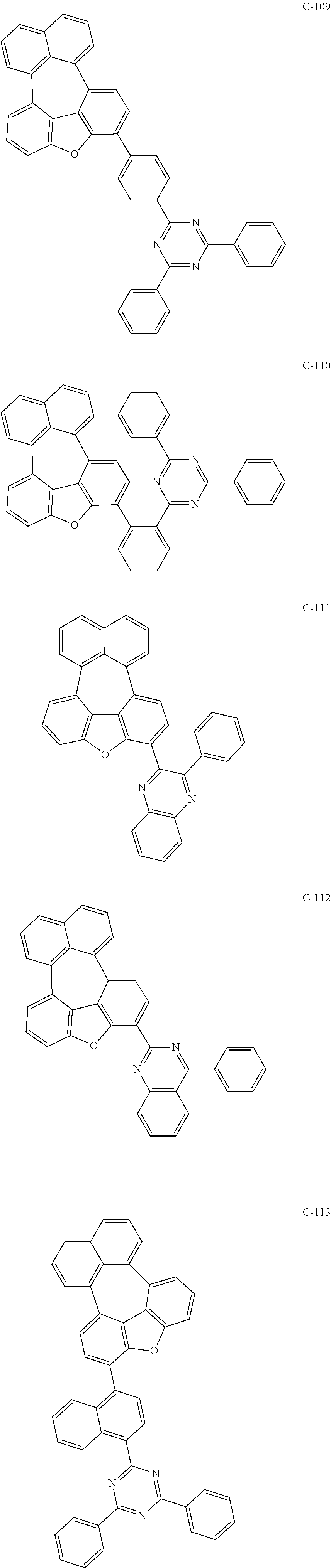 [SMILES: c1ccc(-c2nc(-c3ccccc3)nc(-c3ccc(-c4ccc5c6c4oc4cccc(c46)-c4cccc6cccc-5c46)cc3)n2)cc1 ; c1ccc(-c2nc(-c3ccccc3)nc(-c3ccccc3-c3ccc4c5c3oc3cccc(c35)-c3cccc5cccc-4c35)n2)cc1 ; c1ccc(-c2nc3ccccc3nc2-c2ccc3c4c2oc2cccc(c24)-c2cccc4cccc-3c24)cc1 ; c1ccc(-c2nc(-c3ccc4c5c3oc3cccc(c35)-c3cccc5cccc-4c35)nc3ccccc23)cc1 ; c1ccc(-c2nc(-c3ccccc3)nc(-c3ccc(-c4ccc5c6c4oc4cccc(c46)-c4cccc6cccc-5c46)c4ccccc34)n2)cc1]

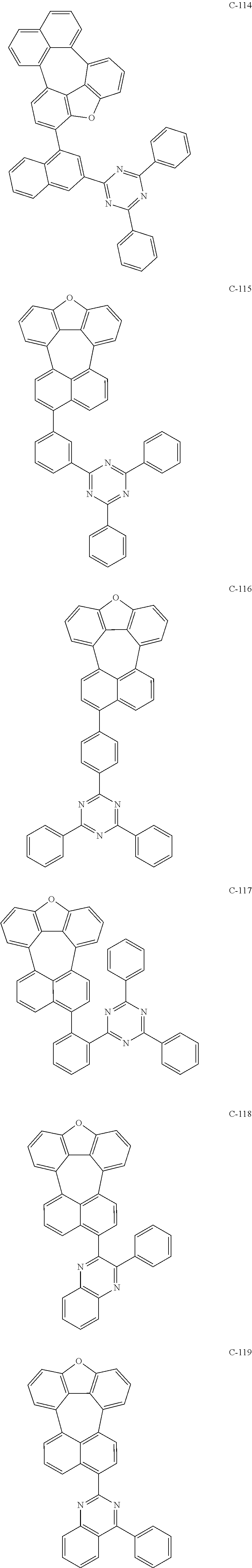 [SMILES: c1ccc(-c2nc(-c3ccccc3)nc(-c3cc(-c4ccc5c6c4oc4cccc(c46)-c4cccc6cccc-5c46)c4ccccc4c3)n2)cc1 ; c1ccc(-c2nc(-c3ccccc3)nc(-c3cccc(-c4ccc5c6c(cccc46)-c4cccc6oc7cccc-5c7c46)c3)n2)cc1 ; c1ccc(-c2nc(-c3ccccc3)nc(-c3ccc(-c4ccc5c6c(cccc46)-c4cccc6oc7cccc-5c7c46)cc3)n2)cc1 ; c1ccc(-c2nc(-c3ccccc3)nc(-c3ccccc3-c3ccc4c5c(cccc35)-c3cccc5oc6cccc-4c6c35)n2)cc1 ; c1ccc(-c2nc3ccccc3nc2-c2ccc3c4c(cccc24)-c2cccc4oc5cccc-3c5c24)cc1 ; c1ccc(-c2nc(-c3ccc4c5c(cccc35)-c3cccc5oc6cccc-4c6c35)nc3ccccc23)cc1]

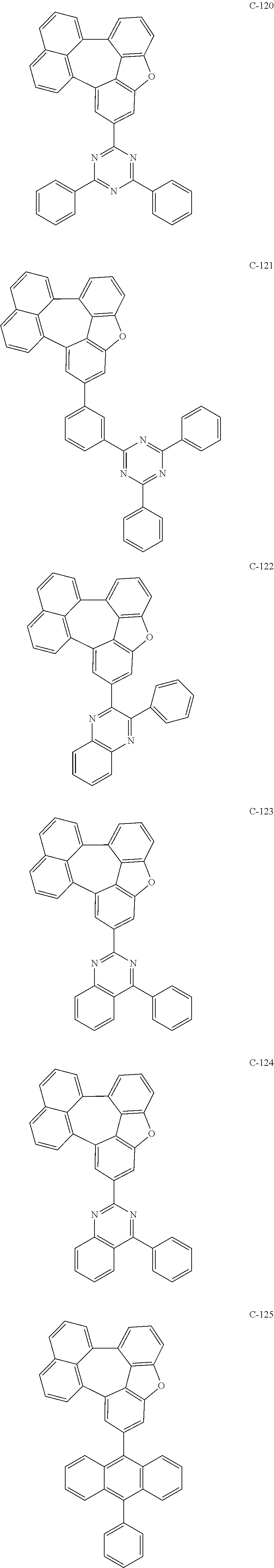

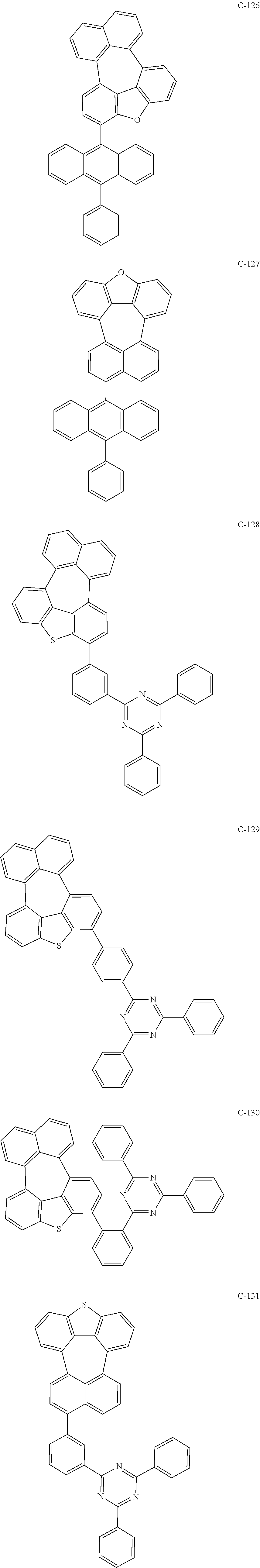 [SMILES: c1ccc(-c2c3ccccc3c(-c3ccc4c5c3oc3cccc(c35)-c3cccc5cccc-4c35)c3ccccc23)cc1 ; c1ccc(-c2c3ccccc3c(-c3ccc4c5c(cccc35)-c3cccc5oc6cccc-4c6c35)c3ccccc23)cc1 ; c1ccc(-c2nc(-c3ccccc3)nc(-c3cccc(-c4ccc5c6c4sc4cccc(c46)-c4cccc6cccc-5c46)c3)n2)cc1 ; c1ccc(-c2nc(-c3ccccc3)nc(-c3ccc(-c4ccc5c6c4sc4cccc(c46)-c4cccc6cccc-5c46)cc3)n2)cc1 ; c1ccc(-c2nc(-c3ccccc3)nc(-c3ccccc3-c3ccc4c5c3sc3cccc(c35)-c3cccc5cccc-4c35)n2)cc1 ; c1ccc(-c2nc(-c3ccccc3)nc(-c3cccc(-c4ccc5c6c(cccc46)-c4cccc6sc7cccc-5c7c46)c3)n2)cc1]

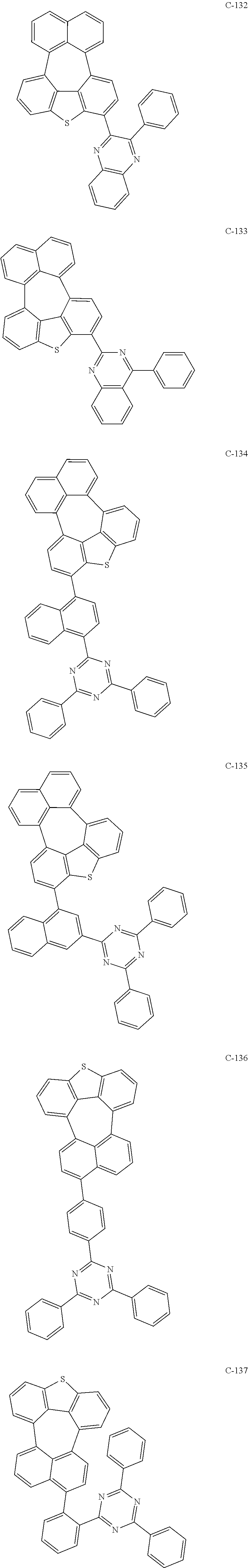 [SMILES: c1ccc(-c2nc3ccccc3nc2-c2ccc3c4c2sc2cccc(c24)-c2cccc4cccc-3c24)cc1 ; c1ccc(-c2nc(-c3ccc4c5c3sc3cccc(c35)-c3cccc5cccc-4c35)nc3ccccc23)cc1 ; c1ccc(-c2nc(-c3ccccc3)nc(-c3ccc(-c4ccc5c6c4sc4cccc(c46)-c4cccc6cccc-5c46)c4ccccc34)n2)cc1 ; c1ccc(-c2nc(-c3ccccc3)nc(-c3cc(-c4ccc5c6c4sc4cccc(c46)-c4cccc6cccc-5c46)c4ccccc4c3)n2)cc1 ; c1ccc(-c2nc(-c3ccccc3)nc(-c3ccc(-c4ccc5c6c(cccc46)-c4cccc6sc7cccc-5c7c46)cc3)n2)cc1 ; c1ccc(-c2nc(-c3ccccc3)nc(-c3ccccc3-c3ccc4c5c(cccc35)-c3cccc5sc6cccc-4c6c35)n2)cc1]

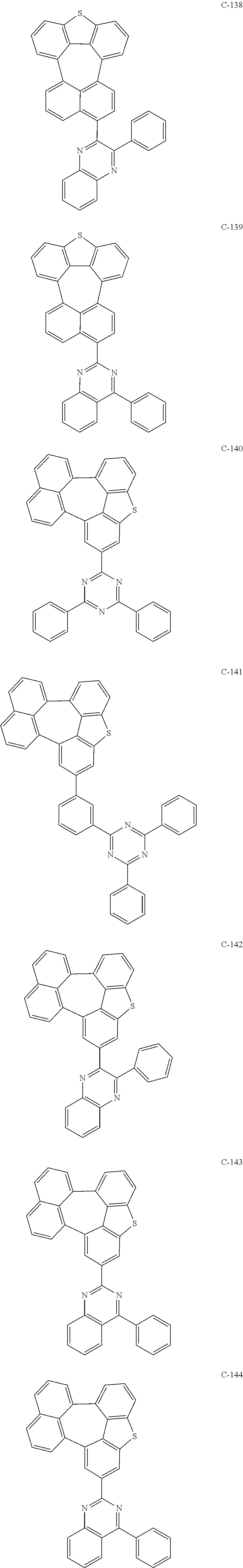

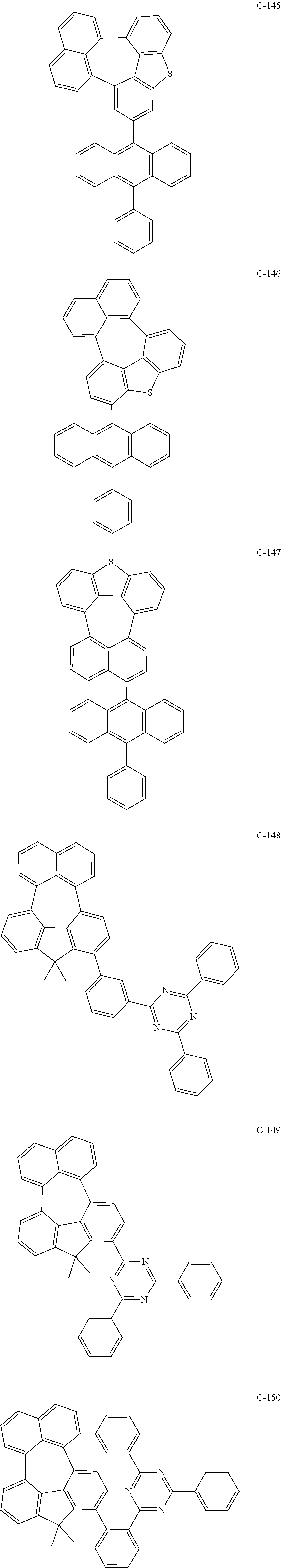

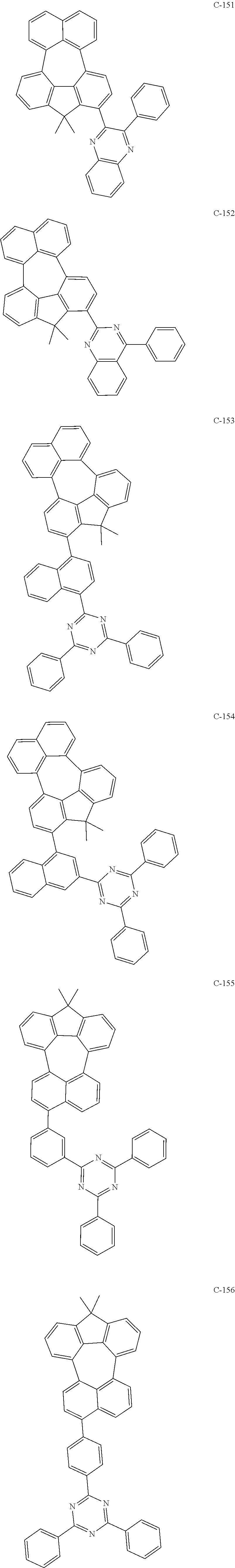

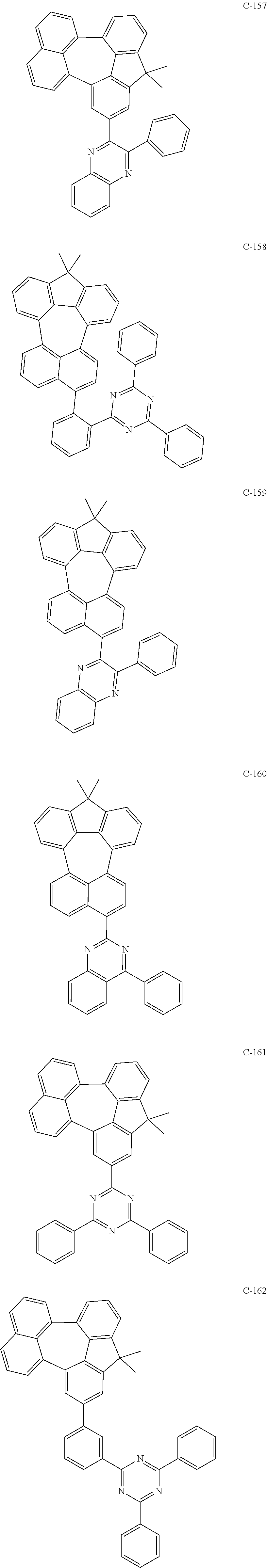 [SMILES: CC1(C)c2cccc3c2-c2c(cc(-c4nc5ccccc5nc4-c4ccccc4)cc21)-c1cccc2cccc-3c12 ; CC1(C)c2cccc3c2-c2c(cccc21)-c1ccc(-c2ccccc2-c2nc(-c4ccccc4)nc(-c4ccccc4)n2)c2cccc-3c12 ; CC1(C)c2cccc3c2-c2c(cccc21)-c1ccc(-c2nc4ccccc4nc2-c2ccccc2)c2cccc-3c12 ; CC1(C)c2cccc3c2-c2c(cccc21)-c1ccc(-c2nc(-c4ccccc4)c4ccccc4n2)c2cccc-3c12 ; CC1(C)c2cccc3c2-c2c(cc(-c4nc(-c5ccccc5)nc(-c5ccccc5)n4)cc21)-c1cccc2cccc-3c12 ; CC1(C)c2cccc3c2-c2c(cc(-c4cccc(-c5nc(-c6ccccc6)nc(-c6ccccc6)n5)c4)cc21)-c1cccc2cccc-3c12]

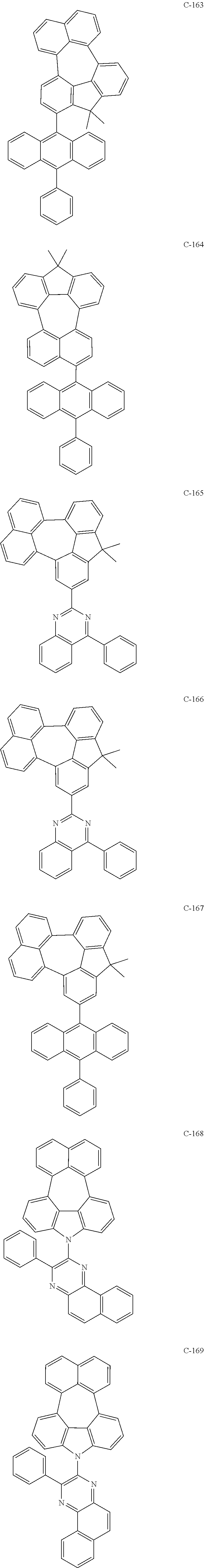

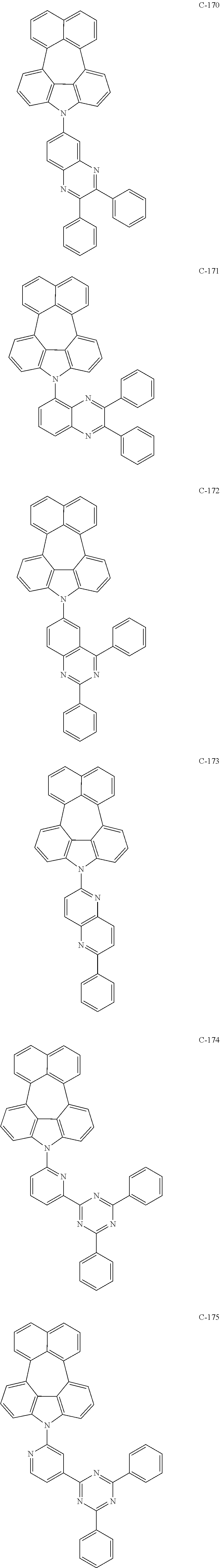

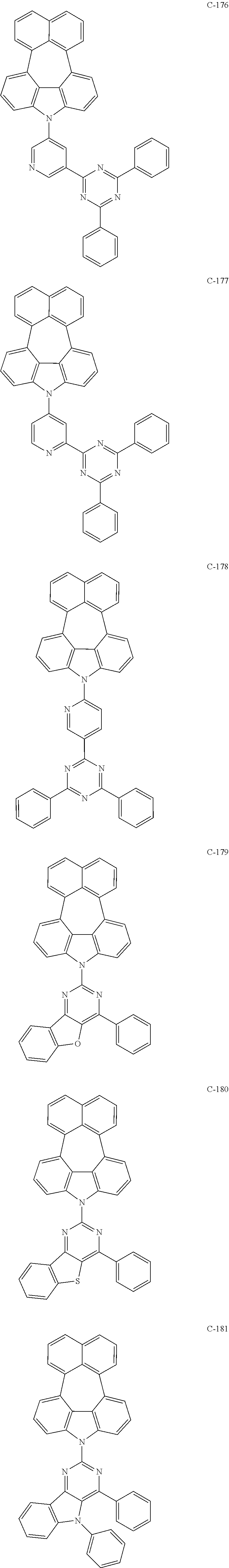 [SMILES: c1ccc(-c2nc(-c3ccccc3)nc(-c3cncc(-n4c5cccc6c5c5c(cccc54)-c4cccc5cccc-6c45)c3)n2)cc1 ; c1ccc(-c2nc(-c3ccccc3)nc(-c3cc(-n4c5cccc6c5c5c(cccc54)-c4cccc5cccc-6c45)ccn3)n2)cc1 ; c1ccc(-c2nc(-c3ccccc3)nc(-c3ccc(-n4c5cccc6c5c5c(cccc54)-c4cccc5cccc-6c45)nc3)n2)cc1 ; c1ccc(-c2nc(-n3c4cccc5c4c4c(cccc43)-c3cccc4cccc-5c34)nc3c2oc2ccccc23)cc1 ; c1ccc(-c2nc(-n3c4cccc5c4c4c(cccc43)-c3cccc4cccc-5c34)nc3c2sc2ccccc23)cc1 ; c1ccc(-c2nc(-n3c4cccc5c4c4c(cccc43)-c3cccc4cccc-5c34)nc3c4ccccc4n(-c4ccccc4)c23)cc1]

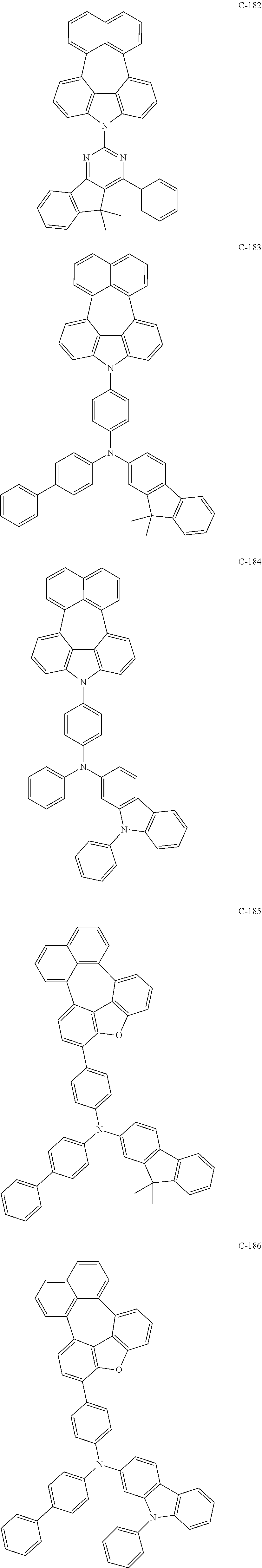

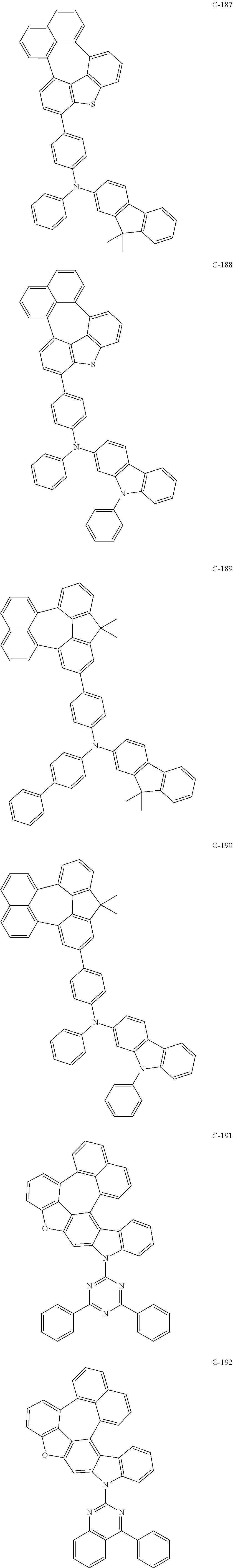

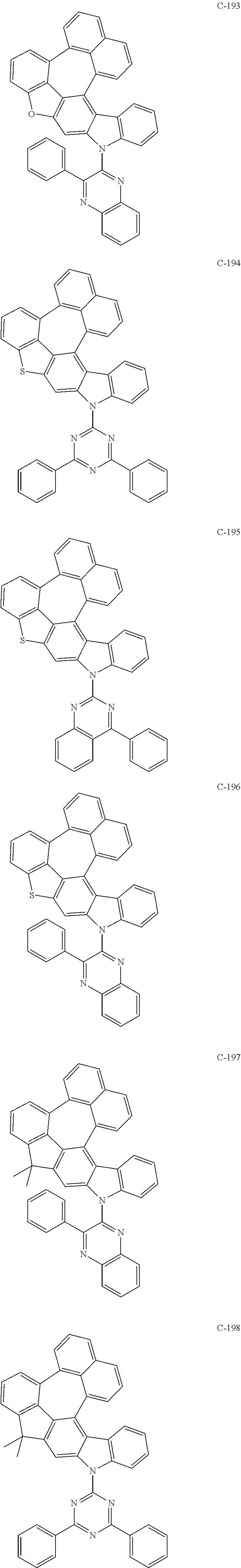

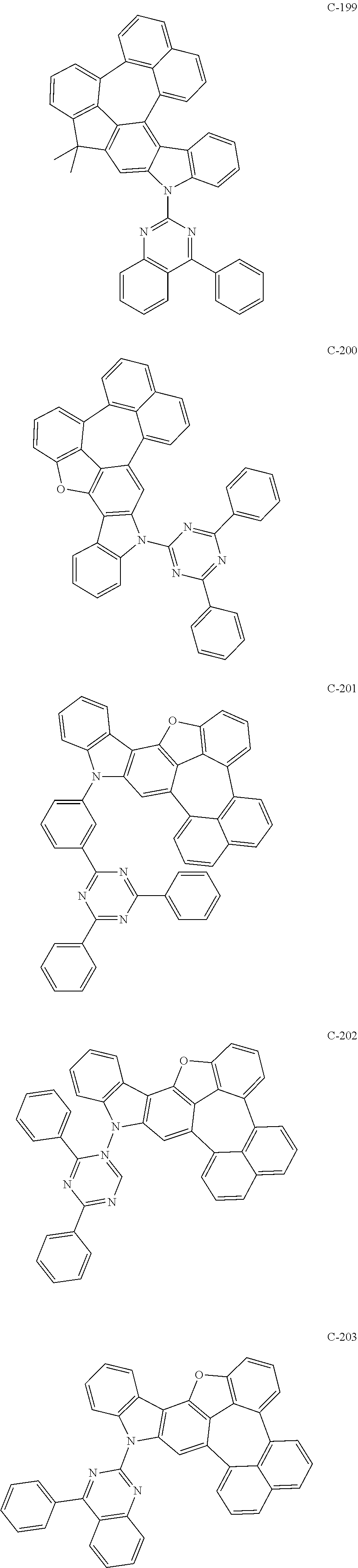

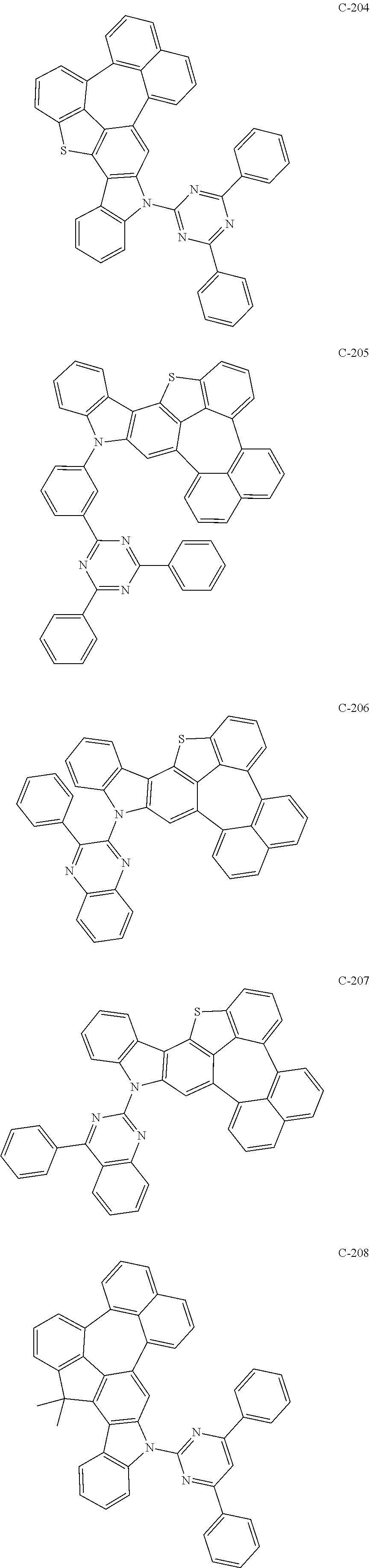

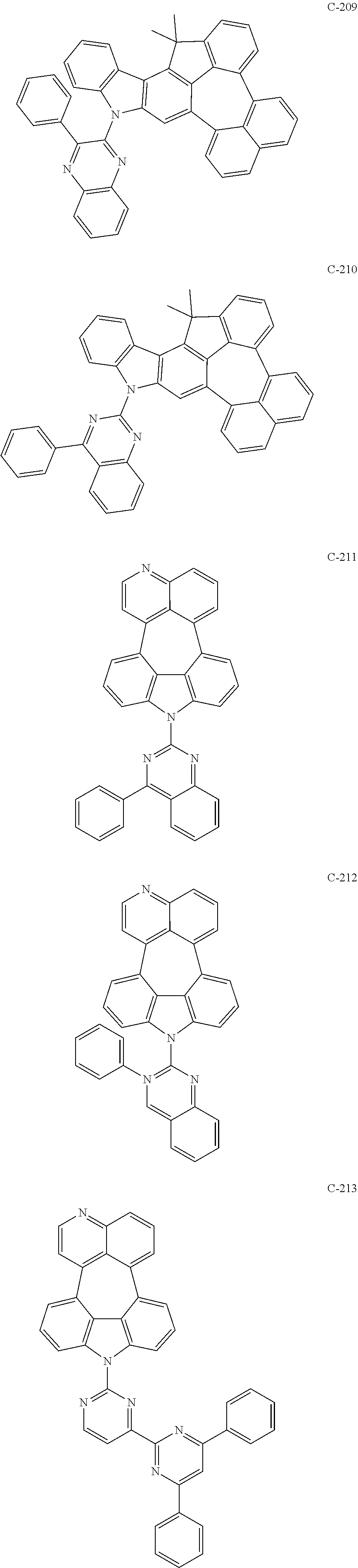

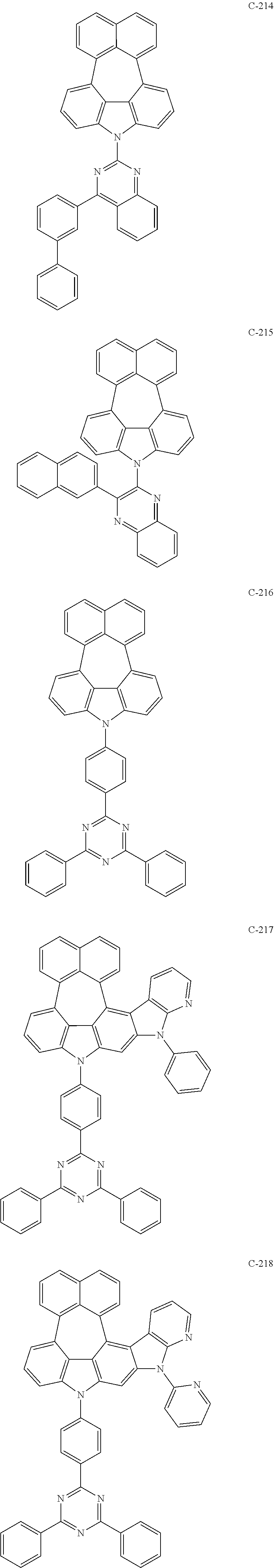 [SMILES: c1ccc(-c2cccc(-c3nc(-n4c5cccc6c5c5c(cccc54)-c4cccc5cccc-6c45)nc4ccccc34)c2)cc1 ; c1ccc2cc(-c3nc4ccccc4nc3-n3c4cccc5c4c4c(cccc43)-c3cccc4cccc-5c34)ccc2c1 ; c1ccc(-c2nc(-c3ccccc3)nc(-c3ccc(-n4c5cccc6c5c5c(cccc54)-c4cccc5cccc-6c45)cc3)n2)cc1 ; c1ccc(-c2nc(-c3ccccc3)nc(-c3ccc(-n4c5cccc6c5c5c(c7c8cccnc8n(-c8ccccc8)c7cc54)-c4cccc5cccc-6c45)cc3)n2)cc1 ; c1ccc(-c2nc(-c3ccccc3)nc(-c3ccc(-n4c5cccc6c5c5c(c7c8cccnc8n(-c8ccccn8)c7cc54)-c4cccc5cccc-6c45)cc3)n2)cc1]

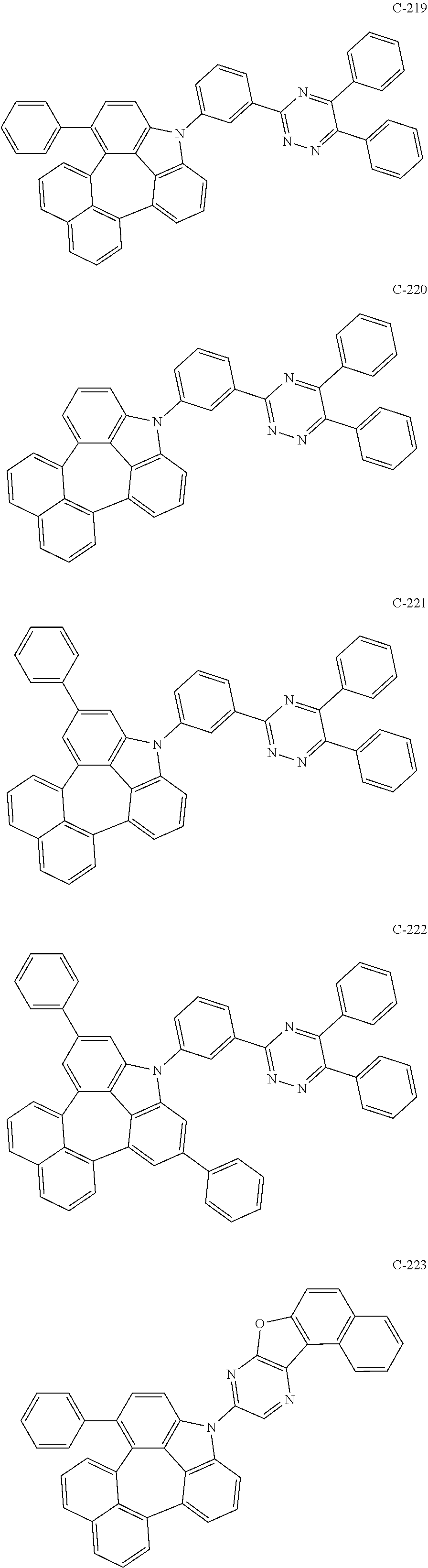 [SMILES: c1ccc(-c2ccc3c4c2-c2cccc5cccc(c25)-c2cccc(c24)n3-c2cccc(-c3nnc(-c4ccccc4)c(-c4ccccc4)n3)c2)cc1 ; c1ccc(-c2nnc(-c3cccc(-n4c5cccc6c5c5c(cccc54)-c4cccc5cccc-6c45)c3)nc2-c2ccccc2)cc1 ; c1ccc(-c2cc3c4c5c(cccc5n(-c5cccc(-c6nnc(-c7ccccc7)c(-c7ccccc7)n6)c5)c4c2)-c2cccc4cccc-3c24)cc1 ; c1ccc(-c2cc3c4c5c(cc(-c6ccccc6)cc5n(-c5cccc(-c6nnc(-c7ccccc7)c(-c7ccccc7)n6)c5)c4c2)-c2cccc4cccc-3c24)cc1 ; c1ccc(-c2ccc3c4c2-c2cccc5cccc(c25)-c2cccc(c24)n3-c2cnc3c(n2)oc2ccc4ccccc4c23)cc1]

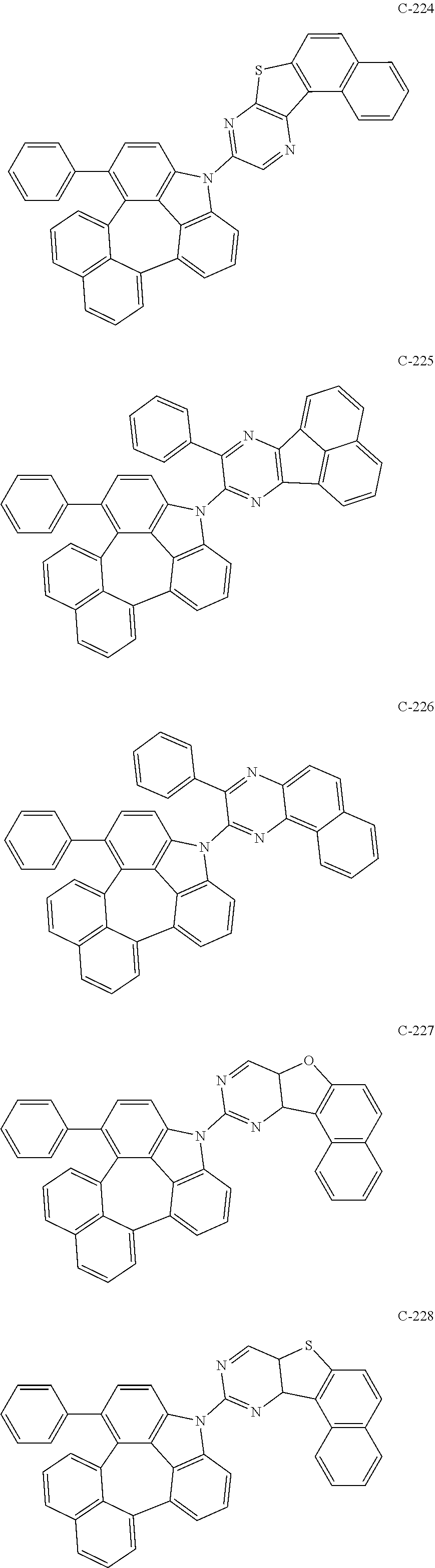 [SMILES: c1ccc(-c2ccc3c4c2-c2cccc5cccc(c25)-c2cccc(c24)n3-c2cnc3c(n2)sc2ccc4ccccc4c23)cc1 ; c1ccc(-c2ccc3c4c2-c2cccc5cccc(c25)-c2cccc(c24)n3-c2nc3c(nc2-c2ccccc2)-c2cccc4cccc-3c24)cc1 ; c1ccc(-c2ccc3c4c2-c2cccc5cccc(c25)-c2cccc(c24)n3-c2nc3c(ccc4ccccc43)nc2-c2ccccc2)cc1 ; C1=NC(n2c3cccc4c3c3c(c(-c5ccccc5)ccc32)-c2cccc3cccc-4c23)=NC2c3c(ccc4ccccc34)OC12 ; C1=NC(n2c3cccc4c3c3c(c(-c5ccccc5)ccc32)-c2cccc3cccc-4c23)=NC2c3c(ccc4ccccc34)SC12]

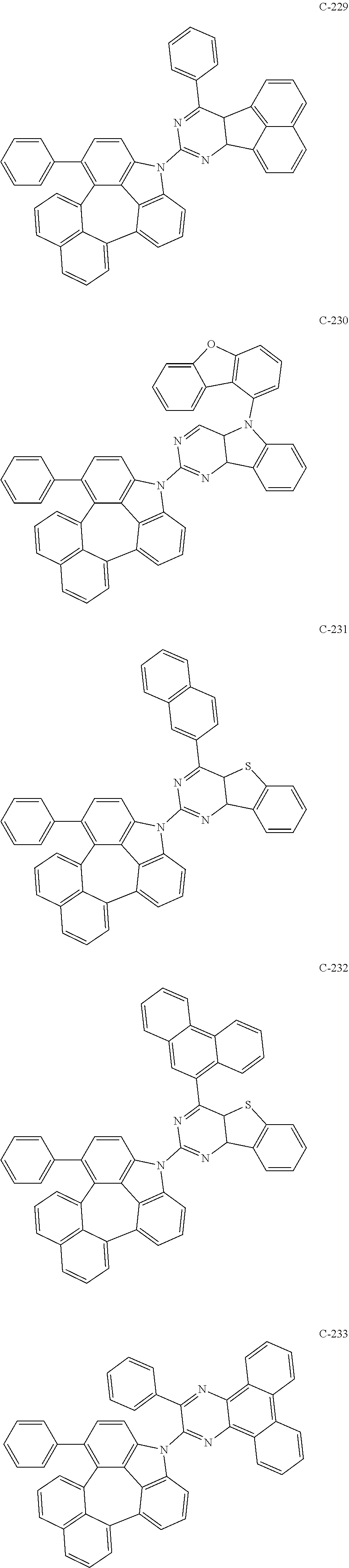 [SMILES: c1ccc(C2=NC(n3c4cccc5c4c4c(c(-c6ccccc6)ccc43)-c3cccc4cccc-5c34)=NC3c4cccc5cccc(c45)C23)cc1 ; C1=NC(n2c3cccc4c3c3c(c(-c5ccccc5)ccc32)-c2cccc3cccc-4c23)=NC2c3ccccc3N(c3cccc4oc5ccccc5c34)C12 ; c1ccc(-c2ccc3c4c2-c2cccc5cccc(c25)-c2cccc(c24)n3C2=NC3c4ccccc4SC3C(c3ccc4ccccc4c3)=N2)cc1 ; c1ccc(-c2ccc3c4c2-c2cccc5cccc(c25)-c2cccc(c24)n3C2=NC3c4ccccc4SC3C(c3cc4ccccc4c4ccccc34)=N2)cc1 ; c1ccc(-c2ccc3c4c2-c2cccc5cccc(c25)-c2cccc(c24)n3-c2nc3c4ccccc4c4ccccc4c3nc2-c2ccccc2)cc1]

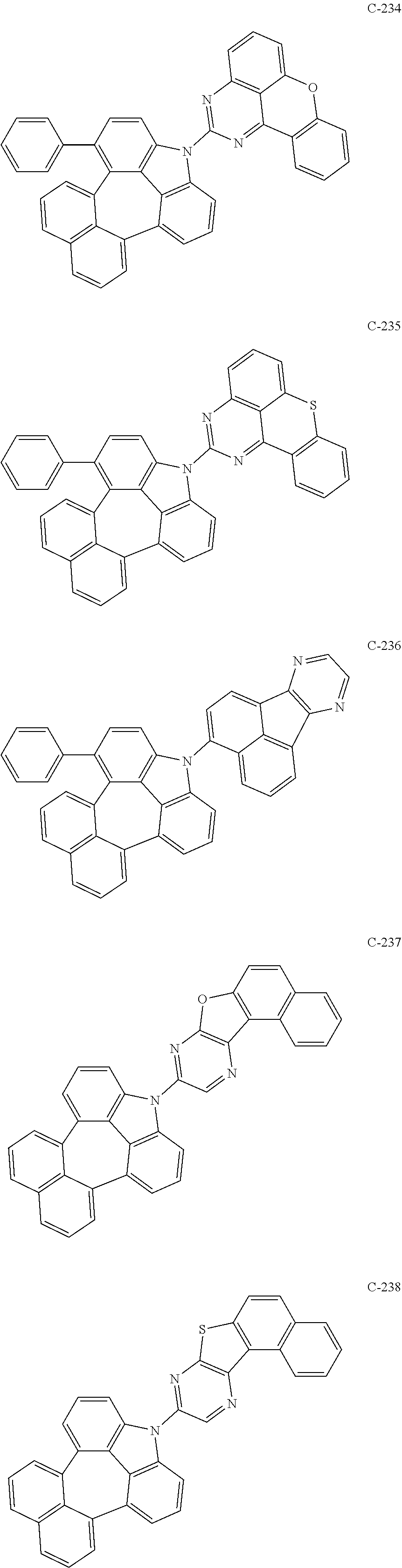 [SMILES: c1ccc(-c2ccc3c4c2-c2cccc5cccc(c25)-c2cccc(c24)n3-c2nc3c4c(cccc4n2)Oc2ccccc2-3)cc1 ; c1ccc(-c2ccc3c4c2-c2cccc5cccc(c25)-c2cccc(c24)n3-c2nc3c4c(cccc4n2)Sc2ccccc2-3)cc1 ; c1ccc(-c2ccc3c4c2-c2cccc5cccc(c25)-c2cccc(c24)n3-c2ccc3c4c(cccc24)-c2nccnc2-3)cc1 ; c1cc2c3c(cccc3c1)-c1cccc3c1c1c-2cccc1n3-c1cnc2c(n1)oc1ccc3ccccc3c12 ; c1cc2c3c(cccc3c1)-c1cccc3c1c1c-2cccc1n3-c1cnc2c(n1)sc1ccc3ccccc3c12]

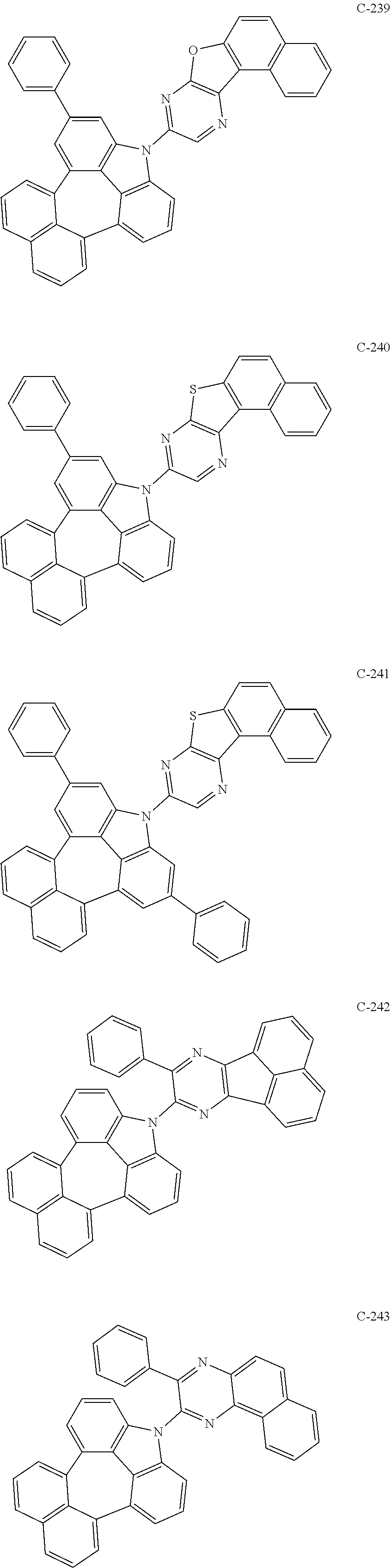 [SMILES: c1ccc(-c2cc3c4c5c(cccc5n(-c5cnc6c(n5)oc5ccc7ccccc7c56)c4c2)-c2cccc4cccc-3c24)cc1 ; c1ccc(-c2cc3c4c5c(cccc5n(-c5cnc6c(n5)sc5ccc7ccccc7c56)c4c2)-c2cccc4cccc-3c24)cc1 ; c1ccc(-c2cc3c4c5c(cc(-c6ccccc6)cc5n(-c5cnc6c(n5)sc5ccc7ccccc7c56)c4c2)-c2cccc4cccc-3c24)cc1 ; c1ccc(-c2nc3c(nc2-n2c4cccc5c4c4c(cccc42)-c2cccc4cccc-5c24)-c2cccc4cccc-3c24)cc1 ; c1ccc(-c2nc3ccc4ccccc4c3nc2-n2c3cccc4c3c3c(cccc32)-c2cccc3cccc-4c23)cc1]

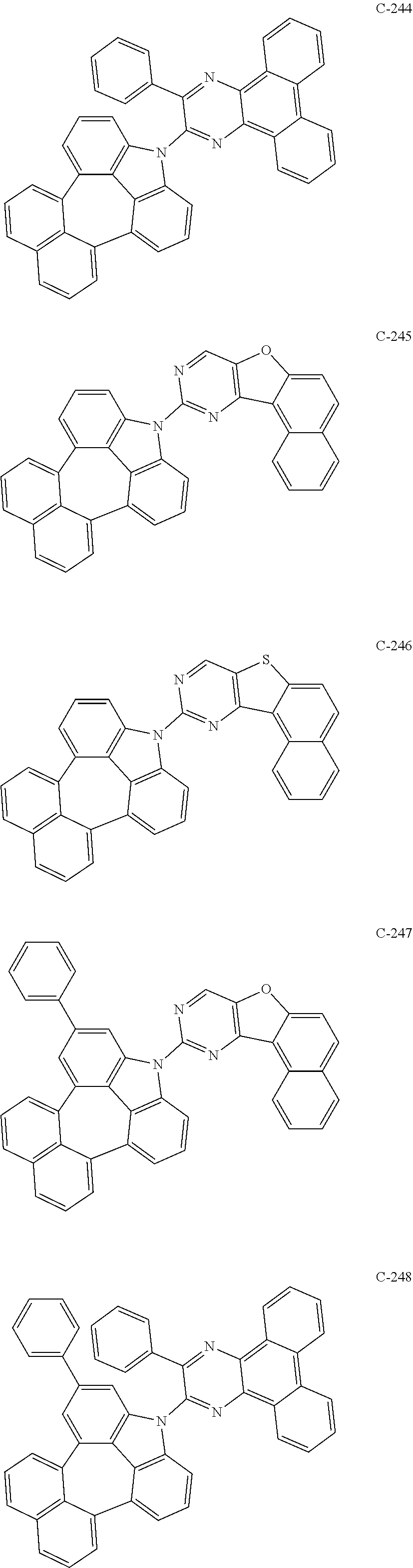 [SMILES: c1ccc(-c2nc3c4ccccc4c4ccccc4c3nc2-n2c3cccc4c3c3c(cccc32)-c2cccc3cccc-4c23)cc1 ; c1cc2c3c(cccc3c1)-c1cccc3c1c1c-2cccc1n3-c1ncc2oc3ccc4ccccc4c3c2n1 ; c1cc2c3c(cccc3c1)-c1cccc3c1c1c-2cccc1n3-c1ncc2sc3ccc4ccccc4c3c2n1 ; c1ccc(-c2cc3c4c5c(cccc5n(-c5ncc6oc7ccc8ccccc8c7c6n5)c4c2)-c2cccc4cccc-3c24)cc1 ; c1ccc(-c2cc3c4c5c(cccc5n(-c5nc6c7ccccc7c7ccccc7c6nc5-c5ccccc5)c4c2)-c2cccc4cccc-3c24)cc1]

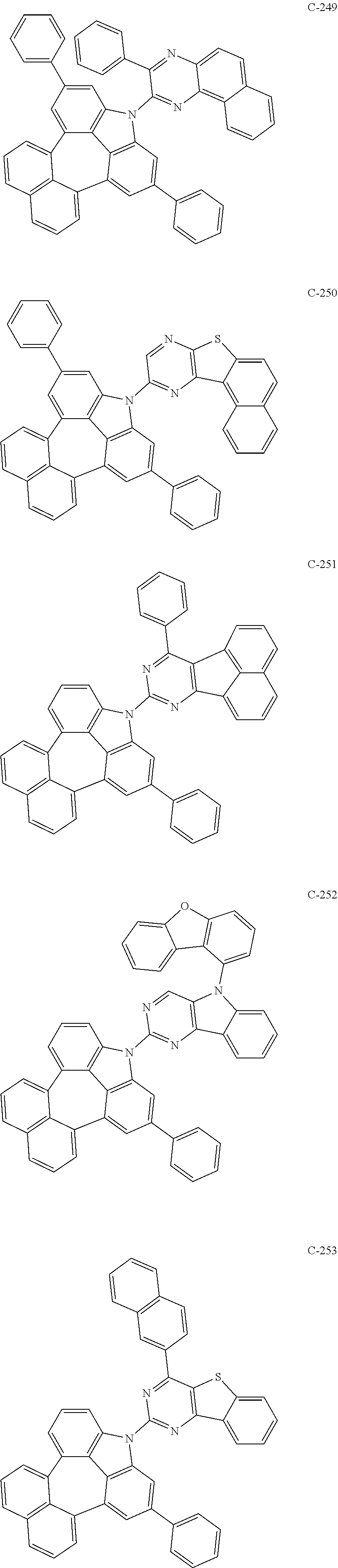 [SMILES: c1ccc(-c2cc3c4c5c(cc(-c6ccccc6)cc5n(-c5nc6c(ccc7ccccc76)nc5-c5ccccc5)c4c2)-c2cccc4cccc-3c24)cc1 ; c1ccc(-c2cc3c4c5c(cc(-c6ccccc6)cc5n(-c5cnc6sc7ccc8ccccc8c7c6n5)c4c2)-c2cccc4cccc-3c24)cc1 ; c1ccc(-c2cc3c4c5c(cccc5n(-c5nc(-c6ccccc6)c6c(n5)-c5cccc7cccc-6c57)c4c2)-c2cccc4cccc-3c24)cc1 ; c1ccc(-c2cc3c4c5c(cccc5n(-c5ncc6c(n5)c5ccccc5n6-c5cccc6oc7ccccc7c56)c4c2)-c2cccc4cccc-3c24)cc1 ; c1ccc(-c2cc3c4c5c(cccc5n(-c5nc(-c6ccc7ccccc7c6)c6sc7ccccc7c6n5)c4c2)-c2cccc4cccc-3c24)cc1]

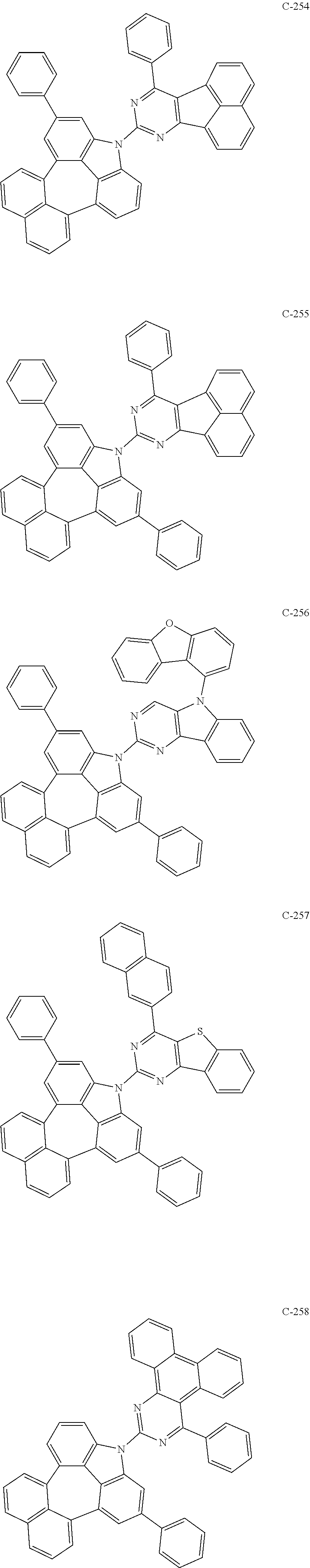

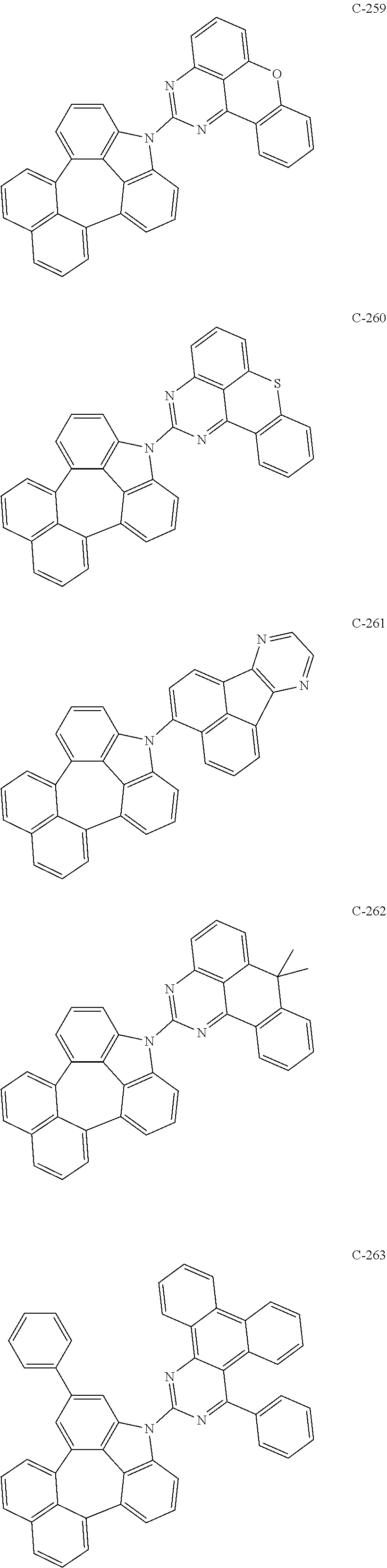 [SMILES: c1ccc2c(c1)Oc1cccc3nc(-n4c5cccc6c5c5c(cccc54)-c4cccc5cccc-6c45)nc-2c13 ; c1ccc2c(c1)Sc1cccc3nc(-n4c5cccc6c5c5c(cccc54)-c4cccc5cccc-6c45)nc-2c13 ; c1cc2c3c(cccc3c1)-c1cccc3c1c1c-2cccc1n3-c1ccc2c3c(cccc13)-c1nccnc1-2 ; CC1(C)c2ccccc2-c2nc(-n3c4cccc5c4c4c(cccc43)-c3cccc4cccc-5c34)nc3cccc1c23 ; c1ccc(-c2cc3c4c5c(cccc5n(-c5nc(-c6ccccc6)c6c7ccccc7c7ccccc7c6n5)c4c2)-c2cccc4cccc-3c24)cc1]

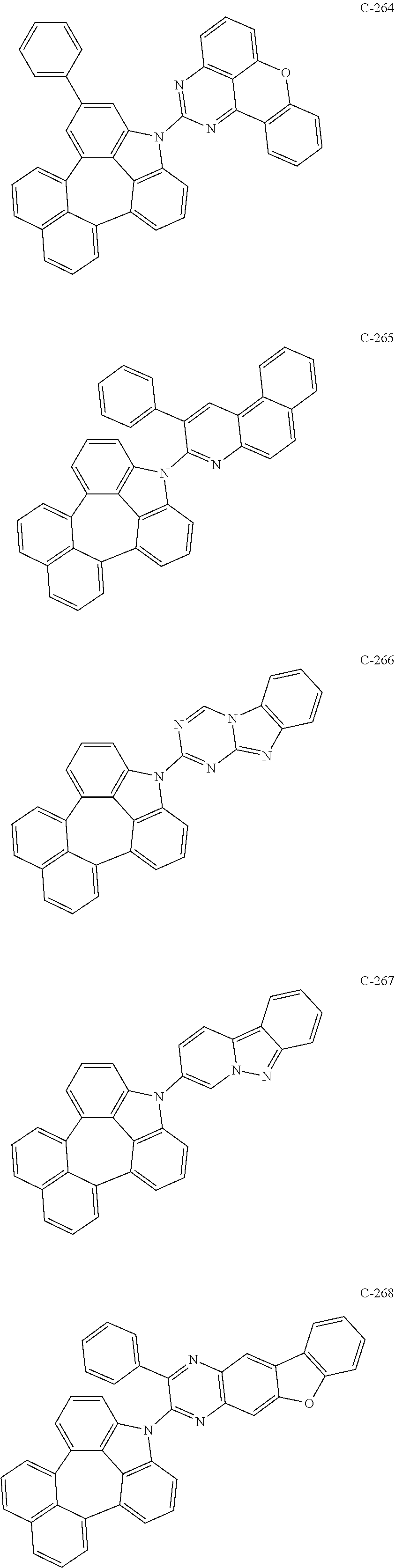 [SMILES: c1ccc(-c2cc3c4c5c(cccc5n(-c5nc6c7c(cccc7n5)Oc5ccccc5-6)c4c2)-c2cccc4cccc-3c24)cc1 ; c1ccc(-c2cc3c(ccc4ccccc43)nc2-n2c3cccc4c3c3c(cccc32)-c2cccc3cccc-4c23)cc1 ; c1cc2c3c(cccc3c1)-c1cccc3c1c1c-2cccc1n3-c1ncn2c(n1)nc1ccccc12 ; c1cc2c3c(cccc3c1)-c1cccc3c1c1c-2cccc1n3-c1ccc2c3ccccc3nn2c1 ; c1ccc(-c2nc3cc4c(cc3nc2-n2c3cccc5c3c3c(cccc32)-c2cccc3cccc-5c23)oc2ccccc24)cc1]

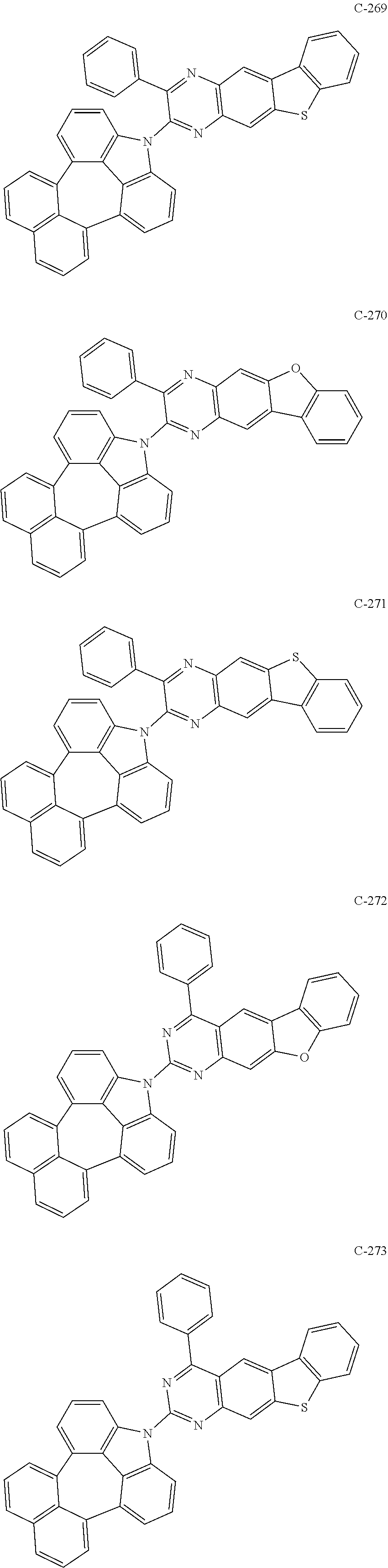 [SMILES: c1ccc(-c2nc3cc4c(cc3nc2-n2c3cccc5c3c3c(cccc32)-c2cccc3cccc-5c23)sc2ccccc24)cc1 ; c1ccc(-c2nc3cc4oc5ccccc5c4cc3nc2-n2c3cccc4c3c3c(cccc32)-c2cccc3cccc-4c23)cc1 ; c1ccc(-c2nc3cc4sc5ccccc5c4cc3nc2-n2c3cccc4c3c3c(cccc32)-c2cccc3cccc-4c23)cc1 ; c1ccc(-c2nc(-n3c4cccc5c4c4c(cccc43)-c3cccc4cccc-5c34)nc3cc4oc5ccccc5c4cc23)cc1 ; c1ccc(-c2nc(-n3c4cccc5c4c4c(cccc43)-c3cccc4cccc-5c34)nc3cc4sc5ccccc5c4cc23)cc1]

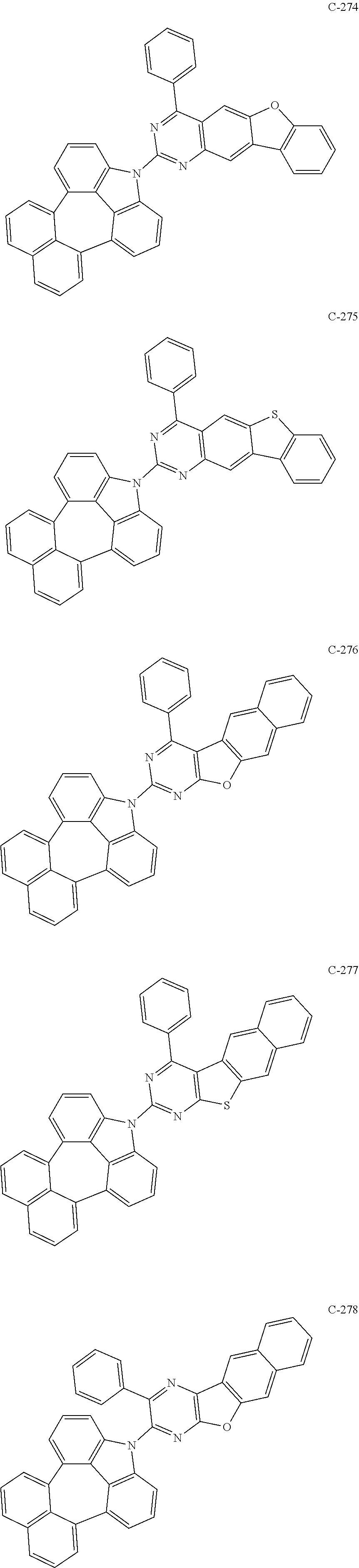 [SMILES: c1ccc(-c2nc(-n3c4cccc5c4c4c(cccc43)-c3cccc4cccc-5c34)nc3cc4c(cc23)oc2ccccc24)cc1 ; c1ccc(-c2nc(-n3c4cccc5c4c4c(cccc43)-c3cccc4cccc-5c34)nc3cc4c(cc23)sc2ccccc24)cc1 ; c1ccc(-c2nc(-n3c4cccc5c4c4c(cccc43)-c3cccc4cccc-5c34)nc3oc4cc5ccccc5cc4c23)cc1 ; c1ccc(-c2nc(-n3c4cccc5c4c4c(cccc43)-c3cccc4cccc-5c34)nc3sc4cc5ccccc5cc4c23)cc1 ; c1ccc(-c2nc3c(nc2-n2c4cccc5c4c4c(cccc42)-c2cccc4cccc-5c24)oc2cc4ccccc4cc23)cc1]

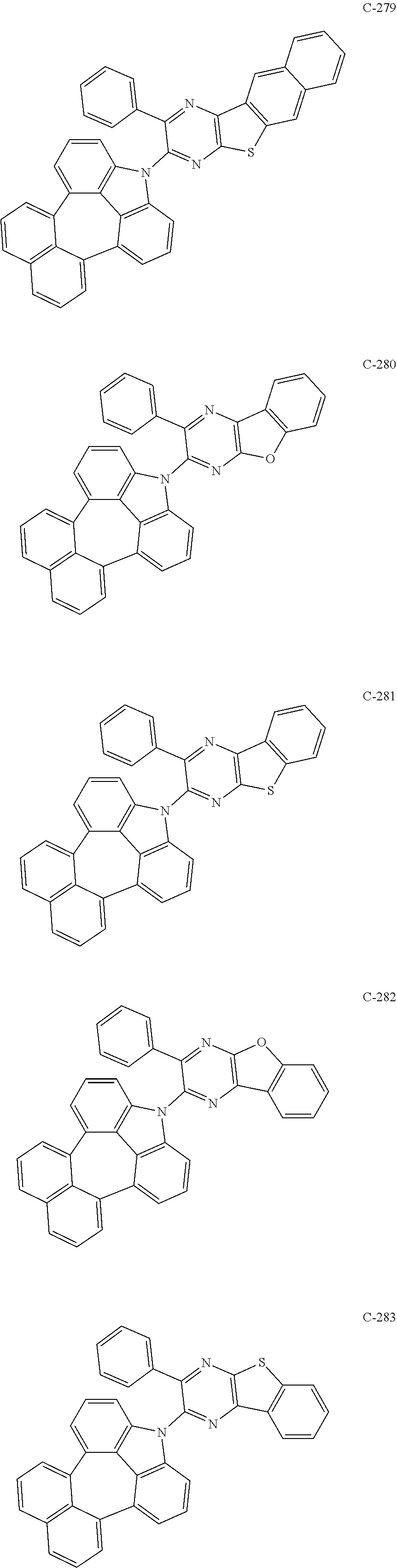 [SMILES: c1ccc(-c2nc3c(nc2-n2c4cccc5c4c4c(cccc42)-c2cccc4cccc-5c24)sc2cc4ccccc4cc23)cc1 ; c1ccc(-c2nc3c(nc2-n2c4cccc5c4c4c(cccc42)-c2cccc4cccc-5c24)oc2ccccc23)cc1 ; c1ccc(-c2nc3c(nc2-n2c4cccc5c4c4c(cccc42)-c2cccc4cccc-5c24)sc2ccccc23)cc1 ; c1ccc(-c2nc3oc4ccccc4c3nc2-n2c3cccc4c3c3c(cccc32)-c2cccc3cccc-4c23)cc1 ; c1ccc(-c2nc3sc4ccccc4c3nc2-n2c3cccc4c3c3c(cccc32)-c2cccc3cccc-4c23)cc1]

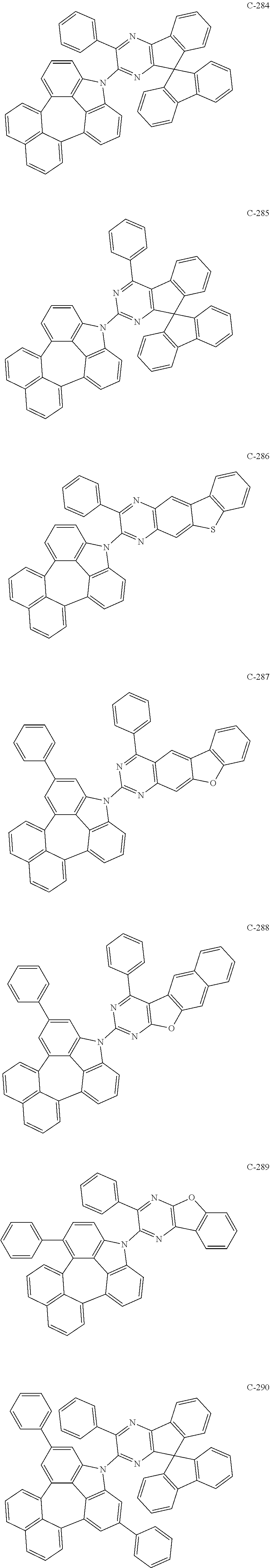

The compound represented by formula 1 according to the present disclosure may be prepared by a synthetic method known to one skilled in the art, and for example may be prepared as shown in the following reaction schemes 1 and 2, but is not limited thereto.

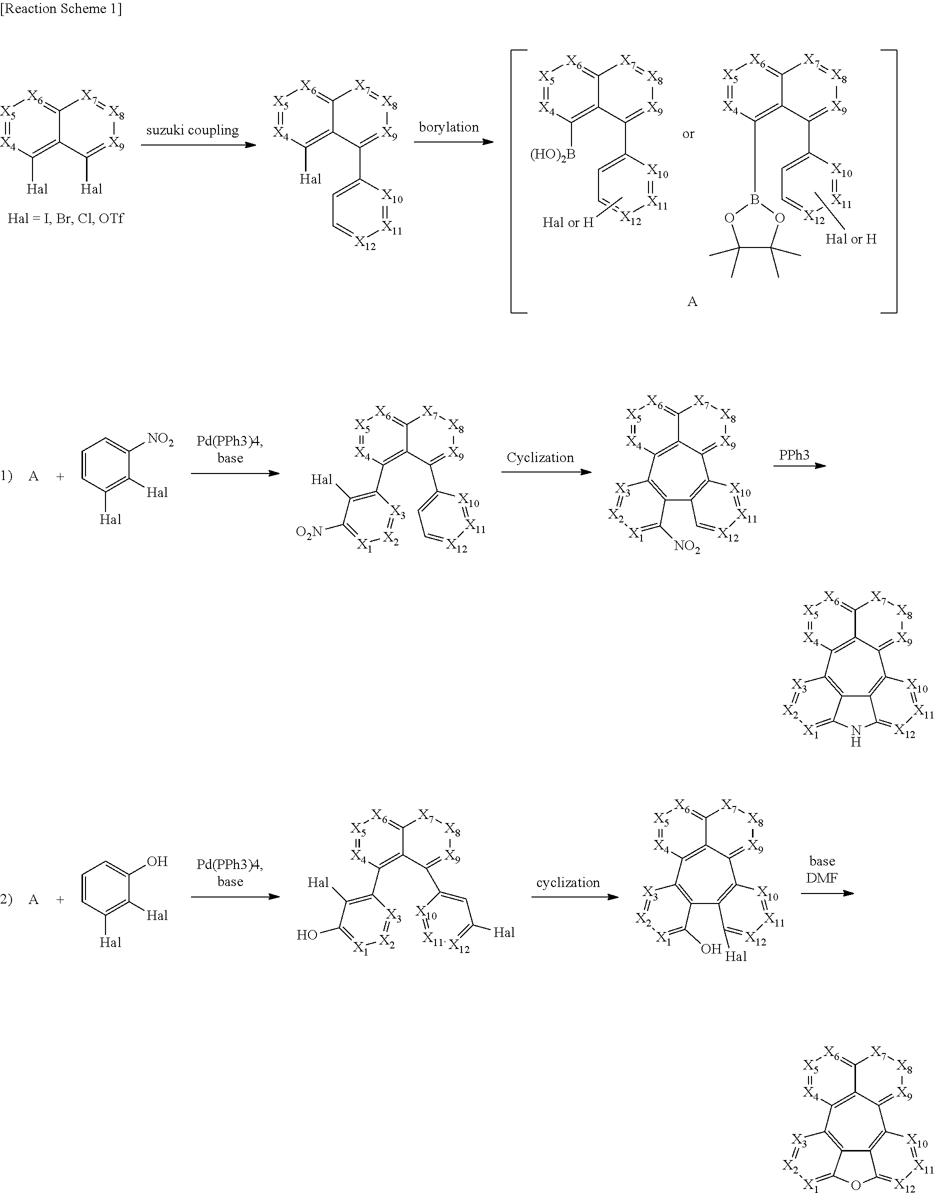

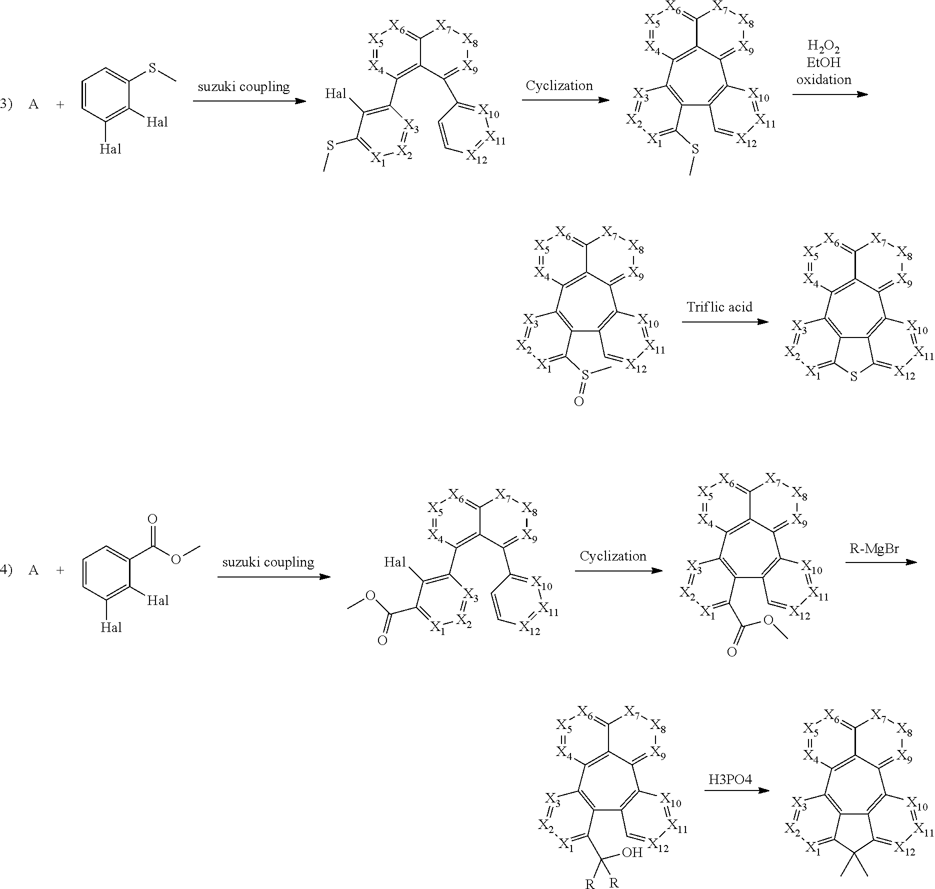

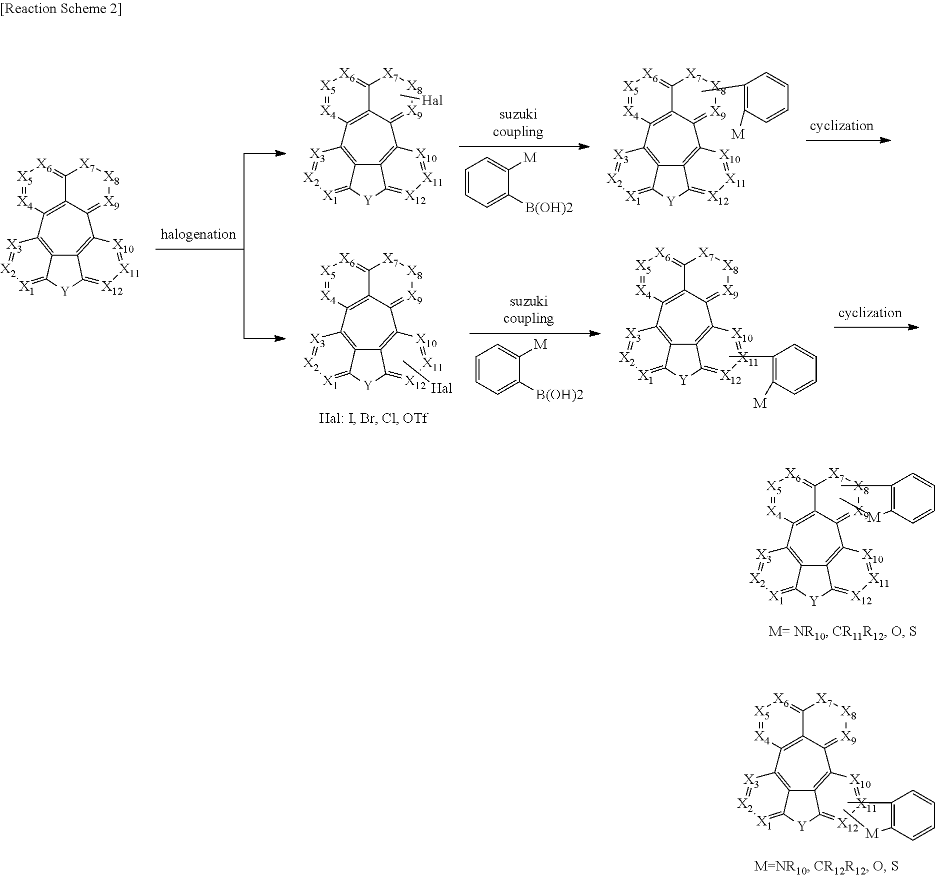

In reaction schemes 1 and 2, X₁ to X₂, Y, and R₁₀ to R₁₂ are as defined in formulas 1,5 and 6.

Although illustrative synthesis examples of the compound represented by formula 1 are described above, one skilled in the art will be able to readily understand that all of them are based on a Buchwald-Hartwig cross-coupling reaction, an N-arylation reaction, a H-mont-mediated etherification reaction, a Miyaura borylation reaction, a Suzuki cross-coupling reaction, an Intramolecular acid-induced cyclization reaction, a Pd(II)-catalyzed oxidative cyclization reaction, a Grignard reaction, a Heck reaction, a Cyclic Dehydration reaction, an SN₁ substitution reaction, an SN₂ substitution reaction, a Phosphine-mediated reductive cyclization reaction, etc., and the reactions above proceed even when substituents, which are defined in formula 1 above but are not specified in the specific synthesis examples, are bonded.

The present disclosure may provide an organic electroluminescent device comprising the compound represented by formula 1. Specifically, the organic electroluminescent device may comprise the compound represented by formula 1, and may further comprise at least one other organic electroluminescent compound.

In addition, the present disclosure may provide an organic electroluminescent material comprising the organic electroluminescent compound of formula 1, and an organic electroluminescent device comprising the organic electroluminescent material. The organic electroluminescent material may consist of the organic electroluminescent compound of the present disclosure as a sole compound, or may further comprise conventional materials generally used in organic electroluminescent materials.

Meanwhile, the organic electroluminescent device of the present disclosure may comprise a first electrode, a second electrode, and at least one organic layer between the first and second electrodes. The organic layer may comprise at least one organic electroluminescent compound of formula 1. The organic layer may further comprise at least one compound selected from the group consisting of arylamine-based compounds and styrylarylamine-based compounds. Also, the organic layer may further comprise at least one metal selected from the group consisting of metals of Group 1, metals of Group 2, transition metals of the 4^{th} period, transition metals of the 5^{th} period, lanthanides, and organic metals of the d-transition elements of the Periodic Table, or at least one complex compound comprising the metal.

One of the first and second electrodes may be an anode, and the other may be a cathode. The first electrode and the second electrode may each be formed with a transmissive conductive material, a transflective conductive material, or a reflective conductive material, The organic electroluminescent device may be a top emission type, a bottom emission type, or both-sides emission type depending on the type of the material forming the first electrode and the second electrode. The organic layer may comprise a light-emitting layer, and may further comprise at least one layer selected from a hole injection layer, a hole transport layer, a hole auxiliary layer, a light-emitting auxiliary layer, an electron transport layer, an electron injection layer, an interlayer, a hole blocking layer, an electron blocking layer, and an electron buffer layer.

The present disclosure may comprise a hole transport zone between an anode and a light-emitting layer, and the hole transport zone may comprise at least one of a hole injection layer, a hole transport layer, a hole auxiliary layer, a light-emitting auxiliary layer and an electron blocking layer. The hole injection layer, the hole transport layer, the hole auxiliary layer, the light-emitting auxiliary layer and the electron blocking layer, respectively, may be a single layer or a plurality of layers in which two or more layers are stacked. The hole injection layer may be multi-layers in order to lower the hole injection barrier (or hole injection voltage) from the anode to the hole transport layer or the electron blocking layer, wherein two compounds may be used simultaneously in each of the multi-layers. The electron blocking layer may be placed between the hole transport layer (or the hole injection layer) and the light-emitting layer, and can confine the excitons within the light-emitting layer by blocking the overflow of electrons from the light-emitting layer to prevent a light-emitting leakage.

In addition, the hole transport zone may comprise a p-doped hole injection layer, a hole transporting layer, and a light-emitting auxiliary layer. Herein, the p-doped hole injection layer means a hole injection layer doped with a p-dopant. The p-dopant is a material capable of imparting p-type semiconductor properties. The p-type semiconductor properties mean the properties of injecting or transporting holes at the HOMO energy level, i.e., the properties of a material having a high hole conductivity.

The present disclosure may comprise an electron transport zone between the light-emitting layer and the cathode. The electron transport zone may comprise at least one of a hole blocking layer, an electron transport layer, an electron buffer layer and an electron injection layer. The hole blocking layer, the electron transport layer, the electron buffer layer, and the electron injection layer, respectively, may be a single layer or a plurality of layers in which two or more layers are stacked. The electron injection layer may be further doped with an n-dopant(s). The electron buffer layer may be multi-layers in order to control the injection of the electron and improve the interfacial properties between the light-emitting layer and the electron injection layer, wherein two compounds may be used simultaneously in each of the multi-layers. The hole blocking layer or the electron transport layer may also be multi-layers, wherein a plurality of compounds may be used in each of the layers.

The light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or the hole transport, or for preventing the overflow of electrons. When the light-emitting auxiliary layer is placed between the cathode and the light-emitting layer, it can be used for promoting the electron injection and/or the electron transport, or for preventing the overflow of holes. Also, the hole auxiliary layer may be placed between the hole transport layer (or hole injection layer) and the light-emitting layer, and may be effective to promote or block the hole transport rate (or the hole injection rate), thereby enabling the charge balance to be controlled. When an organic electroluminescent device includes two or more hole transport layers, the hole transport layer, which is further included, may be used as a hole auxiliary layer or an electron blocking layer. The light-emitting auxiliary layer, the hole auxiliary layer or the electron blocking layer may have an effect of improving the efficiency and/or the lifetime of the organic electroluminescent device.

In the organic electroluminescent device of the present disclosure, preferably, at least one layer selected from a chalcogenide layer, a metal halide layer, and a metal oxide layer (hereinafter, “a surface layer”) may be placed on an inner surface(s) of one or both electrode(s). Specifically, a chalcogenide (including oxides) layer of silicon or aluminum is preferably placed on an anode surface of an electroluminescent medium layer, and a metal halide layer or a metal oxide layer is preferably placed on a cathode surface of an electroluminescent medium layer. The operation stability for the organic electroluminescent device may be obtained by the surface layer, Preferably, the chalcogenide includes SiOₓ(1≤X≤2), AlOₓ(1≤X≤105), SiON, SiAlON, etc.; the metal halide includes LiF, MgF₂, CaF₂, a rare earth metal fluoride, etc.; and the metal oxide includes Cs₂O, Li₂O, MgO, SrO, BaO, CaO, etc.

In the organic electroluminescent device of the present disclosure, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to an electroluminescent medium. Furthermore, the hole transport compound is oxidized to a cation, and thus it becomes easier to inject and transport holes from the mixed region to the electroluminescent medium. Preferably, the oxidative dopant includes various Lewis acids and acceptor compounds, and the reductive dopant includes alkali metals, alkali metal compounds, alkaline earth metals, rare-earth metals, and mixtures thereof. A reductive dopant layer may be employed as a charge generating layer to prepare an organic electroluminescent device having two or more light-emitting layers and emitting white light.

The organic electroluminescent compound represented by formula 1 may be comprised in the light-emitting layer. When used in the light-emitting layer, the organic electroluminescent compound of formula 1 may be comprised as a host material. Preferably, the light-emitting layer may further comprise at least one dopant. If necessary, other compound(s) than the organic electroluminescent compound of formula 1 may be further comprised as a second host material. Herein, the weight ratio of the first host material to the second host material is in the range of 1:99 to 99:1. The second host material can be any known phosphorescent host.

The dopant comprised in the organic electroluminescent device of the present disclosure may be at least one phosphorescent or fluorescent dopant, and is preferably at least one phosphorescent dopant. The phosphorescent dopant material applied to the organic electroluminescent device of the present disclosure is not particularly limited, but may be preferably selected from the metallated complex compounds of iridium (Ir), osmium (Os), copper (Cu), and platinum (Pt), more preferably selected from ortho-metallated complex compounds of iridium (Ir), osmium (Os), copper (Cu), and platinum (Pt), and even more preferably ortho-metallated iridium complex compounds.

In order to form each layer of the organic electroluminescent device of the present disclosure, dry film-forming methods such as vacuum evaporation, sputtering, plasma, and ion plating methods, or wet film-forming methods such as ink jet printing, nozzle printing, slot coating, spin coating, dip coating, and flow coating methods can be used. When using a solvent in a wet film-forming method, a thin film can be formed by dissolving or diffusing materials forming each layer into any suitable solvent such as ethanol, chloroform, tetrahydrofuran, dioxane, etc. The solvent can be any solvent where the materials forming each layer can be dissolved or diffused, and where there are no problems in film-formation capability.

In addition, it is possible to produce a display system or a lighting system by using the organic electroluminescent compound of the present disclosure. Specifically, it is possible to produce a display system, e.g., a display system for smartphones, tablets, notebooks, PCs, TVs, or cars, or a lighting system, e.g., an outdoor or indoor lighting system, by using the organic electroluminescent compound of the present disclosure.

Hereinafter, the preparation method of the compound of the present disclosure, and the properties thereof will be explained in detail with reference to the representative compounds of the present disclosure, However, the present disclosure is not limited to the following examples.

### EXAMPLE 1: PREPARATION OF COMPOUND C-11

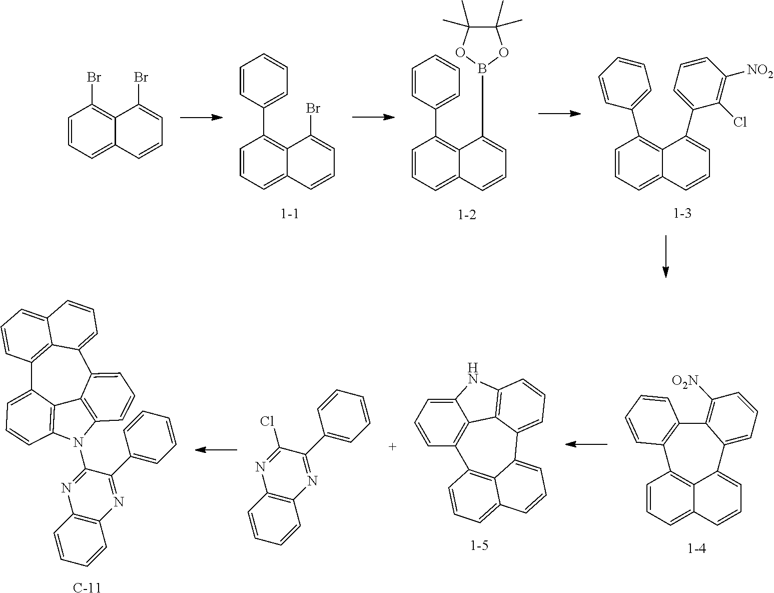

### Synthesis of Compound 1-1

In a flask, 170 g of 1,8-dibromonaphthalene (594 mmol), 60 g of phenylboronic acid (492 mmol), 14.2 g of tetrakis(triphenylphosphine)palladium(0) (12.2 mmol), and 87 g of sodium carbonate (820 mmol) were dissolved in 1.6 L of toluene, 410 mL, of ethanol, and 410 mL of water, and the mixture was refluxed at 120° C. for 3 hours. After completion of the reaction, the organic layer was separated with ethyl acetate and the residual moisture was dried by using magnesium sulfate. The residue was separated by column chromatography to obtain 117 g of compound 1-1 (yield: 84%).

### Synthesis of Compound 1-2

In a flask, 117 g of compound 1-1 (254.2 mmol), 152 g of bis(pinacolato)diboron (598 mmol), 14.5 g of bis(triphenylphosphine)palladium(II) dichloride (20.6 mmol), and 101 g of potassium acetate (1029 mmol) were dissolved in 2 L of 1,4-dioxane, and the mixture was refluxed at 120° C. for 3 hours. After completion of the reaction, the organic layer was separated with ethyl acetate and the residual moisture was dried by using magnesium sulfate. The residue was separated by column chromatography to obtain 85 g of compound 1-2 (yield: 62.5%).

### Synthesis of Compound 1-3

85 g of compound 1-2 (257 mmol), 79 g of 1-bromo-2-chloro-3-nitrobenzene (334 mmol), 17.8 g of tetrakis(triphenylphosphine)palladium(O) (15.4 mmol), 30.8 g of sodium hydroxide (772 mmol), 850 mL of tetrahydrofuran, and 386 mL of water were added to a flask, and the mixture was refluxed at 120° C. for 24 hours. After completion of the reaction, the organic layer was separated with ethyl acetate and the residual moisture was removed by using magnesium sulfate. The residue was separated by column chromatography to obtain 41 g of compound 1-3 (yield: 45%).

### Synthesis of Compound 1-4

45 g of compound 1-3 (125 mmol), 2.87 g of palladium acetate (12.7 mmol), 7.4 g of tricyclohexylphosphine tetrafluoroborate (20.0 mmol), 65.4 g of cesium carbonate (200 mmol) and 822 mL. of dimethylacetamide were added to a flask, and mixture was stirred under reflux for 1 hour. After completion of the reaction, the organic layer was extracted with ethyl acetate and the residual moisture was removed by using magnesium sulfate. The residue was dried and separated by column chromatography to obtain 39.4 g of compound 1-4 (yield: 97%).

### Synthesis of Compound 1-5

In a flask, 39.4 g of compound 164 (121.8 mmol), and 91.7 g of triphenylphosphine (349 mmol) were dissolved in 1.2 L of dichlorobenzene, and the mixture was refluxed at 200° C. for 24 hours. After completion of the reaction, the solvent was removed by distillation under reduced pressure. The residue was separated by column chromatography to obtain 18.5 g of compound 1-5 (yield: 70%).

### Synthesis of Compound C-11

In a flask, 5 g of compound 1-5 (17.1 mmol), 5.03 g of 2-chloro-3-phenylquinoxaline (20.8 mmol), 16.7 g of potassium carbonate (121,1 mmol), and 105 mg of dimethylaminopyridine (0.85 mmol) were dissolved in 100 mL of dimethylformamide, and the mixture was stirred under reflux for 24 hours. After completion of the reaction, the organic layer was extracted with ethyl acetate and the residual moisture was removed by using magnesium sulfate. The residue was dried and separated by column chromatography to obtain 2.1 g of compound C-11 (yield: 25%)

| Compound | MW | Tg | M.P. |
|---|---|---|---|
| C-11 | 495.59 | 123.07° C. | 227° C. |

### EXAMPLE 2: PREPARATION OF COMPOUND C-6

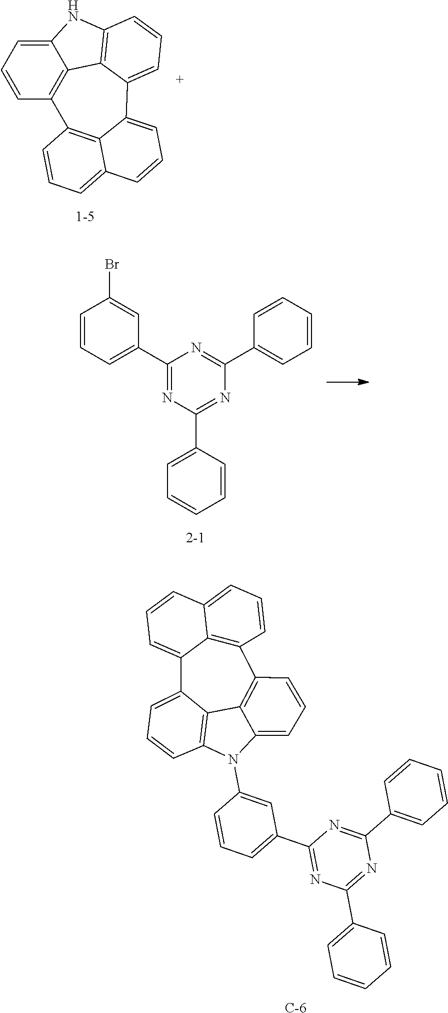

In a flask, 5 g of compound 1-5 (17.1 mmol), 10 g of compound 2-1 (25.7 mmol), 781 mg of tris(dibenzylideneacetone)dipalladium(0) (0.85 mmol), 704 mg of SPhos (1.71 mmol), and 5 g of sodium Cert-butoxide (52 mmol) were dissolved in 86 mL. of xylene, and the mixture was stirred under reflux for 3 hours. After completion of the reaction, the organic layer was extracted with ethyl acetate and the residual moisture was removed. The residue was dried and separated by column chromatography to obtain 4.6 g of compound C-6 (yield: 45%).

| Compound | MW | Tg | M.P. |
|---|---|---|---|
| C-6 | 598.71 | 131.42° C. | 310° C. |

### EXAMPLE 3: PREPARATION OF COMPOUND C-48

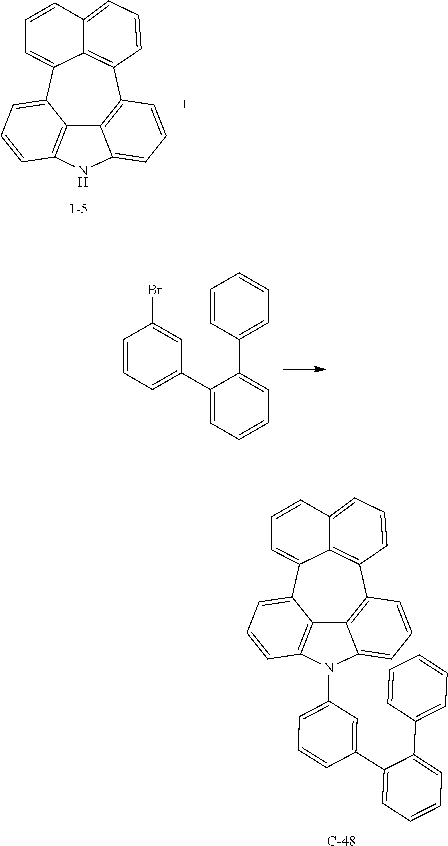

In a flask, 4 g of compound 1-5 (13.72 mmol), 4.2 g of 3-bromo-1,1′:2′,1″-terphenyl (13.72 mmol), 620 mg of tris(dibenzylideneacetone)dipalladium(0) (0.686 mmol), 560 mg of SPhos (1,372 mmol), and 4 g of sodium tert-butoxide (41.18 mmol) were dissolved in 90 mL of xylene, and the mixture was stirred under reflux for 3 hours. After completion of the reaction, the organic layer was extracted with ethyl acetate and the residual moisture was removed by using magnesium sulfate. The residue was dried and separated by column chromatography to obtain 4 g of compound C-48 (yield 56.6%),

| Compound | MW | Tg | M.P. |
|---|---|---|---|
| C-48 | 519.65 | 103.85° C. | 226.8° C. |

Hereinafter, the properties of the organic electroluminescent device (OLED) comprising the compound according to the present disclosure will be explained in detail. However, the following examples merely illustrate the properties of an OLED according to the present disclosure in detail, but the present disclosure is not limited to the following examples.

### Device Example 1: Producing an OLED Comprising the Compound according to the Present Disclosure in an Electron Buffer Layer

An OLED was produced by using the organic electroluminescent compound according to the present disclosure. A transparent electrode indium tin oxide (ITO) thin film (10 Ω/sq) on a glass substrate for an OLED (GEOMATEC CO., LTD., Japan) was subjected to an ultrasonic washing with acetone, ethanol, and distilled water, sequentially, and then was stored in isopropanol. The ITO substrate was then mounted on a substrate holder of a vacuum vapor deposition apparatus. Compound HI-1 was introduced into a cell of the vacuum vapor deposition apparatus, and then the pressure in the chamber of the apparatus was controlled to 10⁻⁶ tom Thereafter, an electric current was applied to the cell to evaporate the above-introduced material, thereby forming a first hole injection layer having a thickness of 60 nm on the ITO substrate. Next, compound HI-2 was introduced into another cell of the vacuum vapor deposition apparatus and was evaporated by applying an electric current to the cell, thereby forming a second hole injection layer having a thickness of 5 nm on the first hole injection layer. Compound HT-1 was then introduced into a cell of the vacuum vapor deposition apparatus and was evaporated by applying an electric current to the cell, thereby forming a first hole transport layer having a thickness of 20 nm on the second hole injection layer. Compound HT-2 was then introduced into another cell of the vacuum vapor deposition apparatus and was evaporated by applying an electric current to the cell, thereby forming a second hole transport layer having a thickness of 5 nm on the first hole transport layer. After forming the hole injection layers and the hole transport layers, a light-emitting layer was formed thereon as follows: Compound BH-3 was introduced into one cell of the vacuum vapor depositing apparatus as a host, and compound BD was introduced into another cell as a dopant. The two materials were evaporated and the dopant was deposited in a doping amount of 2 wt % based on the total amount of the host and dopant to form a light-emitting layer having a thickness of 20 nm on the second hole transport layer. Next, compound C-6 was deposited to form an electron buffer layer having a thickness of 5 nm on the light-emitting layer. Compound ET-1 and compound EI-1 were then introduced into the other two cells and evaporated at the rate of 1:1 to form an electron transport layer having a thickness of 30 nm on the electron buffer layer. After depositing compound EI-1 as an electron injection layer having a thickness of 2 nm on the electron transport layer, an Al cathode having a thickness of 80 nm was deposited on the electron injection layer by another vacuum vapor deposition apparatus. Thus, an OLED was produced.

As a result, a minimum time taken to reduce the luminance from 100% to 90% based on a luminance of 2,000 nit was 187 hours.

### Comparative Example 1: Producing an OLEO not comprising any Electron Buffer Layer

An OLED was produced in the same manner as in Device Example 1, except that compound ET-1 and compound EI-1 were evaporated at the rate of 1:1 to form an electron transport layer having a thickness of 35 nm on the light-ernitting layer, as a device having no electron buffer layer.

As a result, a minimum time taken to reduce the luminance from 100% to 90% based on a luminance of 2,000 nit was 128 hours.

The present inventors have confirmed that the lifetime of the OLED is improved by introducing a separate electron buffer layer other than the electron injection layer and the electron transport layer in order to control the balance of electrons in the light-emitting layer, and comprising the compound of the present disclosure to the electron buffer layer. When the LUMO (Lowest Unoccupied Molecular Orbital) energy level of the host of the light-emitting layer is formed at about −1.629 eV and the LUMO energy level of the electron transport layer is formed at about −1.888 eV, the electrons excessively injected into the light-emitting layer cause a deterioration phenomenon at the interface of the hole transport layer and the light emitting-layer, which causes a decrease in lifetime. In order to solve this deterioration phenomenon, the present inventors incorporated the compound of the present disclosure having a LUMO energy level of about −1.983 eV between the light-emitting layer and the electrontransport layer, As a result, the present inventors have confirmed that the injection of electrons can be efficiently controlled, thereby improving the lifetime of the OLED. By comprising the compound of the present disclosure, the lifetime performance of the blue organic electroluminescent device can be improved. Thus, the blue organic electroluminescent device can exhibit a competitive performance capable of maintaining the balance with the lifetime performance of the red- and green- organic electroluminescent devices, thereby it is expected to be applicable in various fields as well as a display,

### Device Examples 2-1 and 2-2: Producing an OLED comprising the Compound according to the Present Disclosure in a Light-Emitting Layer

OLEDs were produced by using the organic electroluminescent compound according to the present disclosure. A transparent electrode indium tin oxide (ITO) thin film (10 Ω/sq) on a glass substrate for an OLED (GEOMATEC CO. LTD., Japan) was subjected to an ultrasonic washing with acetone, ethanol, and distilled water, sequentially, and then was stored in isopropanol. The ITO substrate was then mounted on a substrate holder of a vacuum vapor deposition apparatus. Compound HI-1 was introduced into a cell of the vacuum vapor deposition apparatus, and then the pressure in the chamber of the apparatus was controlled to 10⁻⁶ torr. Thereafter, an electric current was applied to the cell to evaporate the above-introduced material, thereby forming a first hole injection layer having a thickness of 80 nm on the ITO substrate. Next, compound HI-2 was introduced into another cell of the vacuum vapor deposition apparatus and was evaporated by applying an electric current to the cell, thereby forming a second hole injection layer having a thickness of 5 nm on the first hole injection layer. Compound HT-1 was then introduced into a cell of the vacuum vapor deposition apparatus and was evaporated by applying an electric current to the cell, thereby forming a first hole transport layer having a thickness of 10 nm on the second hole injection layer. Compound HT-3 was then introduced into another cell of the vacuum vapor deposition apparatus and was evaporated by applying an electric current to the cell, thereby forming a second hole transport layer having a thickness of 60 nm on the first hole transport layer. After forming the hole injection layers and the hole transport layers, a light-emitting layer was formed thereon as follows: The compound shown as the first host in Table 1 was introduced into one cell of the vacuum vapor depositing apparatus as a host, and compound D-1 was introduced into another cell as a dopant. The two materials were evaporated at a different rate and the dopant was deposited in a doping amount of 3 wt % based on the total amount of the host and dopant to form a light-emitting layer having a thickness of 40 nm on the second hole transport layer. Next, compound ET-1 and compound EI-1 were then introduced into the other two cells and evaporated at the rate of 1:1 to form an electron transport layer having a thickness of 35 nm on the light-emitting layer. After depositing compound EI-1 as an electron injection layer having a thickness of 2 nm on the electron transport layer, an Al cathode having a thickness of 80 nm was deposited on the electron injection layer by another vacuum vapor deposition apparatus. Thus, an OLED was produced.

### Device Example 2-3: Producing an OLED comprising the Compound according to the Present Disclosure in a light-Emitting Layer

An OLED was produced in the same manner as in Device Example 2-1, except that the first and second host compounds shown in Table 1 below were introduced into two cells of the vacuum vapor depositing apparatus as hosts, and compound D-1 was introduced into another cell, and the two host materials were evaporated at a rate of 1:1 and the dopant material was simultaneously evaporated at a different rate and the dopant was deposited in a doping amount of 3 wt % based on the total amount of the hosts and dopant to form a light-emitting layer.

### Comparative Example 2: Producing an OLED comprising the Conventional Compound in a Light-Emitting Layer

An OLED was produced in the same manner as in Device Example 2-1, except for using compound A as a host of the light-emitting layer.

The driving voltage at a luminance of 1,000 nit, and the time taken for luminance to decrease from 100% to 95% (lifetime; T95) at a luminance of 5,000 nit of the OLEDs produced in Device Examples 2-1 to 2-3 and Comparative Example 2 are provided in Table 1 below.

TABLE 1

| | | | Driving | |
|---|---|---|---|---|
| | | Second | Voltage | Lifetime |
| | First Host | Host | [V] | (T95) [hr] |
| Comparative | A | — | 9.0 | 0.24 |
| Example 2 | | | | |
| Device Example | C-11 | — | 3.6 | 0.5 |
| 2-1 | | | | |
| Device Example | C-6 | — | 3.3 | 0.7 |
| 2-2 | | | | |
| Device Example | C-48 | B | 3.5 | 18.1 |
| 2-3 | | | | |

From Table 1, it can be confirmed that the OLEDs comprising the compound according to the present disclosure as a host material exhibit a lower driving voltage and longer lifetime properties compared to the conventional OLEDs.

The compounds used in the Device Examples and the Comparative Examples are shown in Table 2 below.

TABLE 2

| | | |
|---|---|---|
| Hole Injection Layer/ Hole Transport Layer | 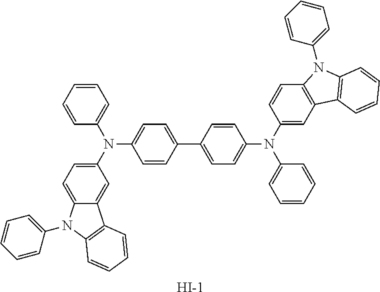 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; [I-]] | 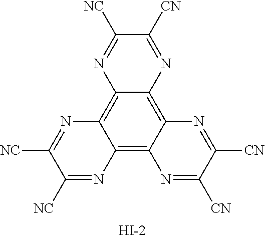 |
| | 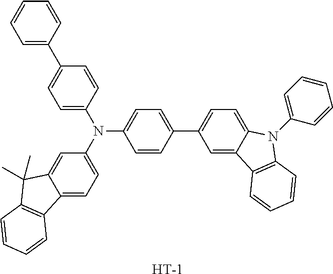 | 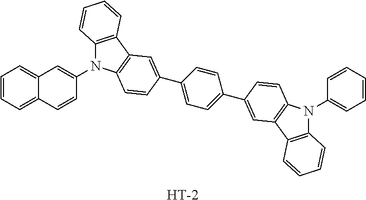 |
| | 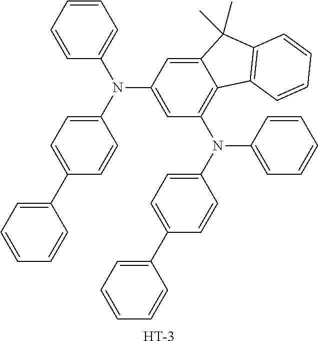 | |
| Light-Emitting Layer | 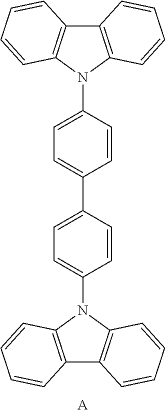 | 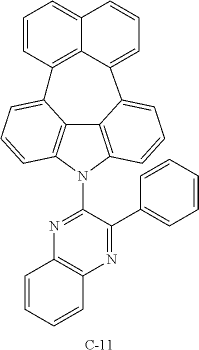 |
| | 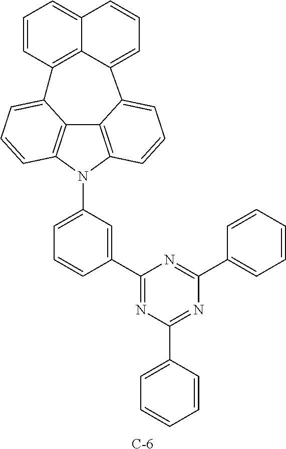 | 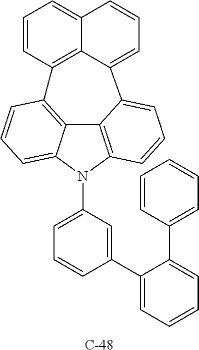 |
| | 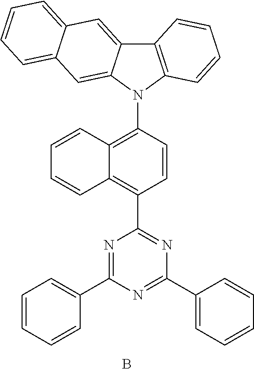 | |
| | 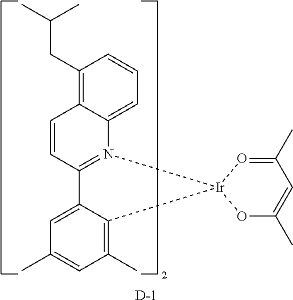 | 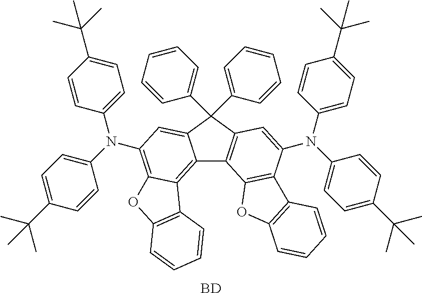 |
| | 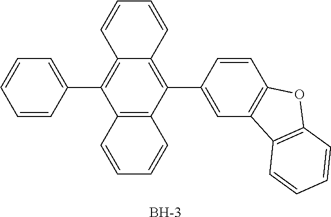 | |
| Electron Transport Layer/Electron Injection Layer | 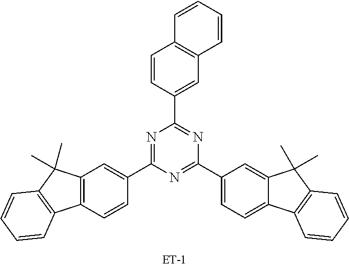 [SMILES: CC1(C)c2ccccc2-c2ccc(-c3nc(-c4ccc5c(c4)C(C)(C)c4ccccc4-5)nc(-c4ccc5ccccc5c4)n3)cc21] | 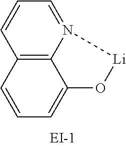 |

1. An organic electroluminescent compound represented by the following formula 1: 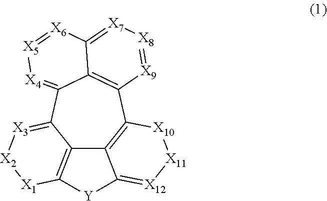
wherein
Y represents —N(R₁)—, —C(R₂)(R₃)—, —O—or —S—;
X₁ to X₁₂, each independently, represent N or CR₄;
R₁ represents —L—(Ar)ₐ;
L represents a single bond, a substituted or unsubstituted (C1-C30)alkylene, a substituted or unsubstituted (C6-C30)arylene, a substituted or unsubstituted (3- to 30-membered) heteroarylene, or a substituted or unsubstituted (C3-C30)cycloalkylene;
Ar, each independently, represents hydrogen, deuterium, a halogen, a cyano, a substituted or unsubstituted (C1-C30)alkyl, a substituted or unsubstituted (C6-C30) aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30) alkyl(C6-C30)arylsilyl, a substituted or unsubstituted (C1-C30)alkyldi(C6-C30) arylsilyl, a substituted or unsubstituted tri(C6-C30)arylsilyl, a substituted or unsubstituted mono- or di- (C1-C30)alkylamino, a substituted or unsubstituted mono- or di- (C6-C30)arylamino, a substituted or unsubstituted (C1-C30)alkyl(C6-C30) arylamino, or a substituted or unsubstituted (C6-C30)aryl(3- to 30-membered) heteroarylamino;
R₂ to R₄, each independently, represent hydrogen, deuterium, a halogen, a cyano, a substituted or unsubstituted (C1-C30)alkyl, a substituted or unsubstituted (C6-C30) aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30) alkyl(C6-C30)arylsilyl, a substituted or unsubstituted (C1-C30)alkyldi(C6-C30) arylsilyl, a substituted or unsubstituted tri(C6-C30)arylsilyl, a substituted or unsubstituted mono- or di- (C1-C30)alkylamino, a substituted or unsubstituted mono- or di- (C6-C30)arylamino, or a substituted or unsubstituted (C1-C30)alkyl(C6-C30) arylamino; or R2 and R3 may be linked to each other to form a ring(s), or at least two of adjacent R₄'s may be linked to each other to form a ring(s); R₂ and R₃ may be the same or different, and each of R₄ may be the same or different;
with the proviso that when both X₉ and X₁₀ represent CR₄, R₄'s in X₉ and X₁₀ are not fused with each other to form a pyrrole ring, a thiophene ring, or a furan ring;
the heteroaryl(ene) contains at least one heteroatom selected from B, N, O, S, Si, and P; and
a represents an integer of 1 or 2; where a is an integer of 2, each of Ar may be the same or different.

2. The organic electroluminescent compound according to claim 1, wherein the substituents of the substituted alkyl(ene), the substituted aryl(ene), the substituted heteroaryl(ene), the substituted cycloalkyl(ene), the substituted alkoxy, the substituted trialkylsilyl, the substituted dialkylarylsilyl, the substituted alkyldiarylsilyl, the substituted triarylsilyl, the substituted mono- or di- alkylamino, the substituted mono- or di- arylamino, the substituted alkylarylamino, and the substituted arylheteroarylamino, each independently, are at least one selected from the group consisting of deuterium; a halogen; a cyano; a carboxyl; a nitro; a hydroxyl; a (C1-C30) alkyl; a halo(C1-C30)alkyl; a (C2-C30)alkenyl; a (C2-C30)alkynyl; a (C1-C30) alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered) heteroaryl unsubstituted or substituted with a (C6-C30)aryl(s); a (C6-C30) aryl unsubstituted or substituted with at least one of a (C1-C30)alkyl(s) and a (3- to 30-membered)heteroaryl(s); a tri(C1-C30)alkylsilyl; a tri(C6-C30)arylsilyl; a di(C1-C30) alkyl(C6-C30)arylsilyl; a (C1-C30)alkyldi(C6-C30)arylsilyl; an amino; a mono- or di- (C1-C30)alkylamino; a mono- or di- (C6-C30)arylamino unsubstituted or substituted with a (C1-C30)alkyl(s); a (C1-C30)alkyl(C6-C30)arylamino; a (C6-C30) aryl(3- to 30-membered)heteroarylamino unsubstituted or substituted with a (C6-C30) aryl(s); a (C1-C30)alkylcarbonyl; a (C1-C30)alkoxycarbonyl; a (C6-C30) arylcarbonyl; a di(C6-C30)arylboronyl; a di(C1-C30)alkylboronyl; a (C1-C30) alkyl(C6-C30)arylboronyl; a (C6-C30)aryl(C1-C30)alkyl; and a (C1-C30)alkyl(C6-C30) aryl.

3. The organic electroluminescent compound according to claim 1, wherein Ar represents a substituted or unsubstituted phenyl, a substituted or unsubstituted naphthyl, a substituted or unsubstituted biphenyl, a substituted or unsubstituted terphenyl, a substituted or unsubstituted fluorenyl, a substituted or unsubstituted anthracenyl, a substituted or unsubstituted fluoranthenyl, a substituted or unsubstituted triazinyl, a substituted or unsubstituted pyridyl, a substituted or unsubstituted pyrimidinyl, a substituted or unsubstituted benzofuropyrimidinyl, a substituted or unsubstituted indenopyrimidinyl, a substituted or unsubstituted benzothienopyrimidinyl, a substituted or unsubstituted acenaphthopyrimidinyl, a substituted or unsubstituted pyrimidoindole, a substituted or unsubstituted quinazolinyl, a substituted or unsubstituted benzoquinazolinyl, a substituted or unsubstituted quinoxalinyl, a substituted or unsubstituted benzoquinoxalinyl, a substituted or unsubstituted dibenzoquinoxalinyl, a substituted or unsubstituted quinolyl, a substituted or unsubstituted benzoquinolyl, a substituted or unsubstituted isoquinolyl, a substituted or unsubstituted benzoisoquinolyl, a substituted or unsubstituted benzothienoquinolyl, a substituted or unsubstituted benzofuroquinolyl, a substituted or unsubstituted triazolyl, a substituted or unsubstituted pyrazolyl, a substituted or unsubstituted carbazolyl, a substituted or unsubstituted dibenzothiophenyl, a substituted or unsubstituted benzothiophenyl, a substituted or unsubstituted dibenzofuranyl, a substituted or unsubstituted benzofuranyl, a substituted or unsubstituted naphthyridinyl, a substituted or unsubstituted benzothiophenyl, a substituted or unsubstituted phenanthroimidazolyl, a substituted or unsubstituted diphenylamino, a substituted or unsubstituted phenylbiphenylamino, a substituted or unsubstituted fluorenylphenylamino, a substituted or unsubstituted fluorenylbiphenylamino, a substituted or unsubstituted carbazolylphenylamino, a substituted or unsubstituted dibenzothiophenylphenylamino, a substituted or unsubstituted dibenzofuranylphenylamino, a substituted or unsubstituted benzofuropyrazinyl, a substituted or unsubstituted benzofuroquinoxalinyl, a substituted or unsubstituted benzothienoquinoxalinyl, a substituted or unsubstituted benzothienoquinazolinyl, a substituted or unsubstituted benzofuroquinazolinyl, a substituted or unsubstituted benzothienopyrazinyl, a substituted or unsubstituted naphthofuropyrazinyl, a substituted or unsubstituted naphthothienopyrazinyl, a substituted or unsubstituted naphthofuropyrimidinyl, a substituted or unsubstituted naphthothienopyrimidinyl, a substituted or unsubstituted spiro[fluorene-indenopyrimidin]yl, a substituted or unsubstituted spiro[fluorene-indenopyrazin]yl, a substituted or unsubstituted acenaphthopyrazinyl, a substituted or unsubstituted benzimidazotriazinyl, a substituted or unsubstituted pyridoindazolyi, a substituted or unsubstituted dibenzoquinazolinyl, a substituted or unsubstituted chromenoquinazolinyl, a substituted or unsubstituted thiochromenoquinazolinyl, or a substituted or unsubstituted dimethyl benzoperimidinyl.

4. The organic electroluminescent compound according to claim 1, wherein two of adjacent X₁ to X₁₂ are CR₄, and two adjacent R₄'s are fused in the form of any one of the following formulas 2 to 6 to form a ring, and the ring may present one or more in one compound represented by formula 1: 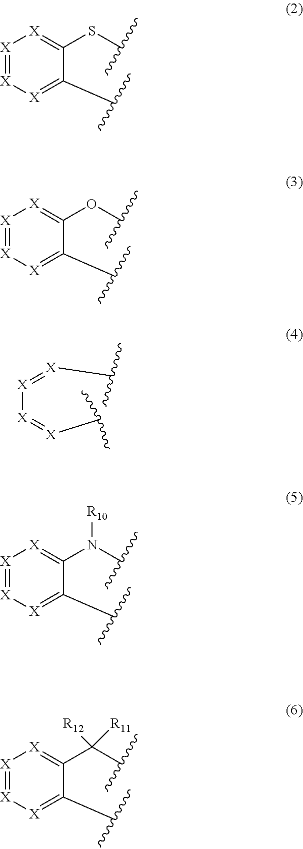
wherein,
X, each independently, represents N or CH;
R₁₀ represents hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30) alkyl(C6-C30)arylsilyl, a substituted or unsubstituted (C1-C30)alkyldi(C6-C30) arylsilyl, a substituted or unsubstituted tri(C6-C30)arylsilyl, a substituted or unsubstituted mono- or di- (C1-C30)alkylamino, a substituted or unsubstituted mono- or di- (C6-C30)arylam no, or a substituted or unsubstituted (C1-C30)alkyl(C6-C30) arylamino;
R₁₁ and R₁₂, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, or a substituted or unsubstituted (C3-C30)cycloalkyl; or R₁₁ and R₁₂ may be linked to each other to form a ring(s); and

[symbol]

5. The organic electroluminescent compound according to claim 1, wherein the compound represented by formula 1 is selected from the group consisting of the following compounds:

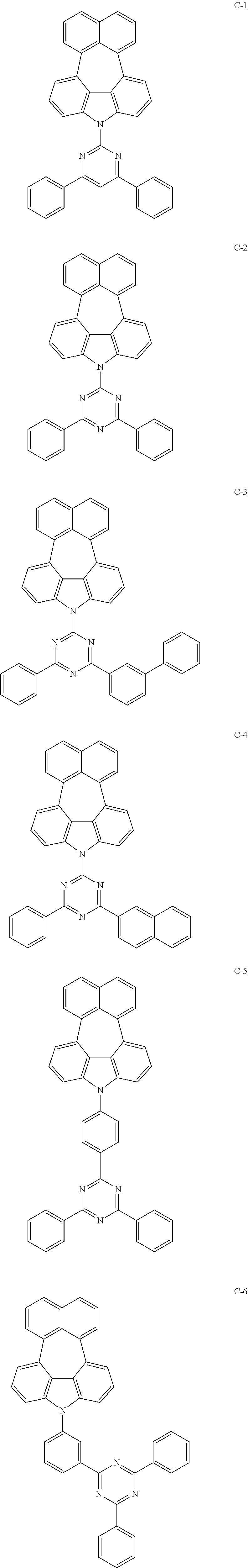 [SMILES: c1ccc(-c2cc(-c3ccccc3)nc(-n3c4cccc5c4c4c(cccc43)-c3cccc4cccc-5c34)n2)cc1 ; c1ccc(-c2nc(-c3ccccc3)nc(-n3c4cccc5c4c4c(cccc43)-c3cccc4cccc-5c34)n2)cc1 ; c1ccc(-c2cccc(-c3nc(-c4ccccc4)nc(-n4c5cccc6c5c5c(cccc54)-c4cccc5cccc-6c45)n3)c2)cc1 ; c1ccc(-c2nc(-c3ccc4ccccc4c3)nc(-n3c4cccc5c4c4c(cccc43)-c3cccc4cccc-5c34)n2)cc1 ; c1ccc(-c2nc(-c3ccccc3)nc(-c3ccc(-n4c5cccc6c5c5c(cccc54)-c4cccc5cccc-6c45)cc3)n2)cc1 ; c1ccc(-c2nc(-c3ccccc3)nc(-c3cccc(-n4c5cccc6c5c5c(cccc54)-c4cccc5cccc-6c45)c3)n2)cc1]

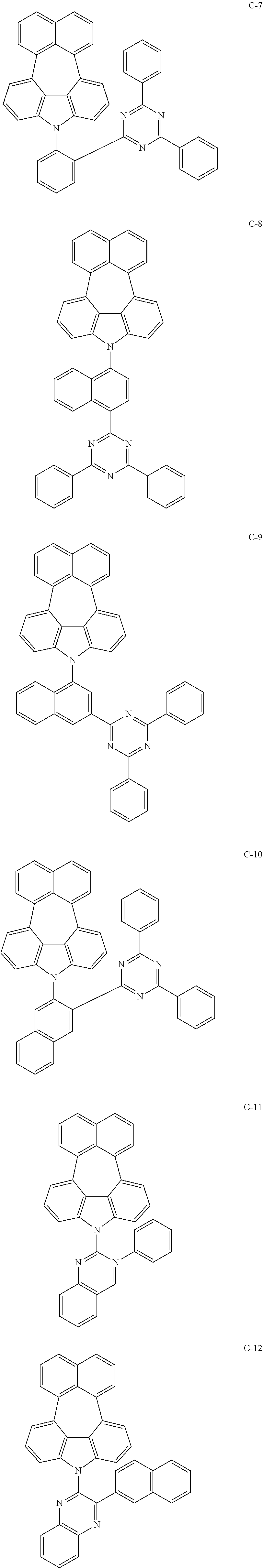

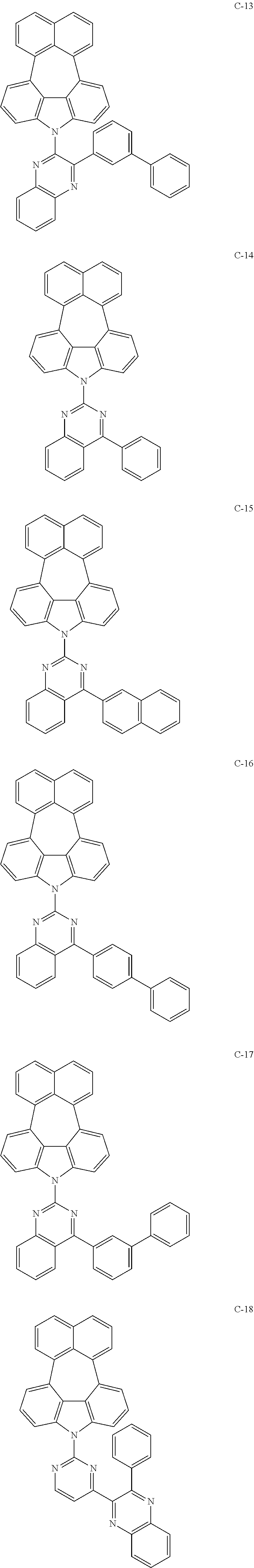 [SMILES: c1ccc(-c2cccc(-c3nc4ccccc4nc3-n3c4cccc5c4c4c(cccc43)-c3cccc4cccc-5c34)c2)cc1 ; c1ccc(-c2nc(-n3c4cccc5c4c4c(cccc43)-c3cccc4cccc-5c34)nc3ccccc23)cc1 ; c1ccc2cc(-c3nc(-n4c5cccc6c5c5c(cccc54)-c4cccc5cccc-6c45)nc4ccccc34)ccc2c1 ; c1ccc(-c2ccc(-c3nc(-n4c5cccc6c5c5c(cccc54)-c4cccc5cccc-6c45)nc4ccccc34)cc2)cc1 ; c1ccc(-c2cccc(-c3nc(-n4c5cccc6c5c5c(cccc54)-c4cccc5cccc-6c45)nc4ccccc34)c2)cc1 ; c1ccc(-c2nc3ccccc3nc2-c2ccnc(-n3c4cccc5c4c4c(cccc43)-c3cccc4cccc-5c34)n2)cc1]

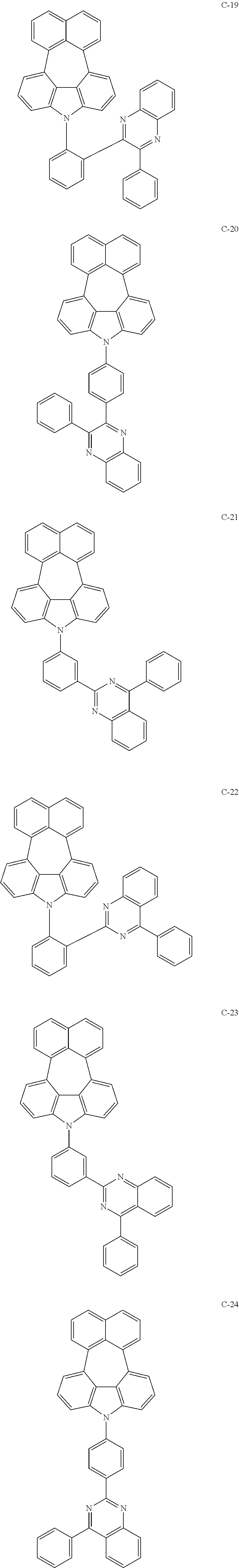 [SMILES: c1ccc(-c2nc3ccccc3nc2-c2ccccc2-n2c3cccc4c3c3c(cccc32)-c2cccc3cccc-4c23)cc1 ; c1ccc(-c2nc3ccccc3nc2-c2ccc(-n3c4cccc5c4c4c(cccc43)-c3cccc4cccc-5c34)cc2)cc1 ; c1ccc(-c2nc(-c3cccc(-n4c5cccc6c5c5c(cccc54)-c4cccc5cccc-6c45)c3)nc3ccccc23)cc1 ; c1ccc(-c2nc(-c3ccccc3-n3c4cccc5c4c4c(cccc43)-c3cccc4cccc-5c34)nc3ccccc23)cc1 ; c1ccc(-c2nc(-c3cccc(-n4c5cccc6c5c5c(cccc54)-c4cccc5cccc-6c45)c3)nc3ccccc23)cc1 ; c1ccc(-c2nc(-c3ccc(-n4c5cccc6c5c5c(cccc54)-c4cccc5cccc-6c45)cc3)nc3ccccc23)cc1]

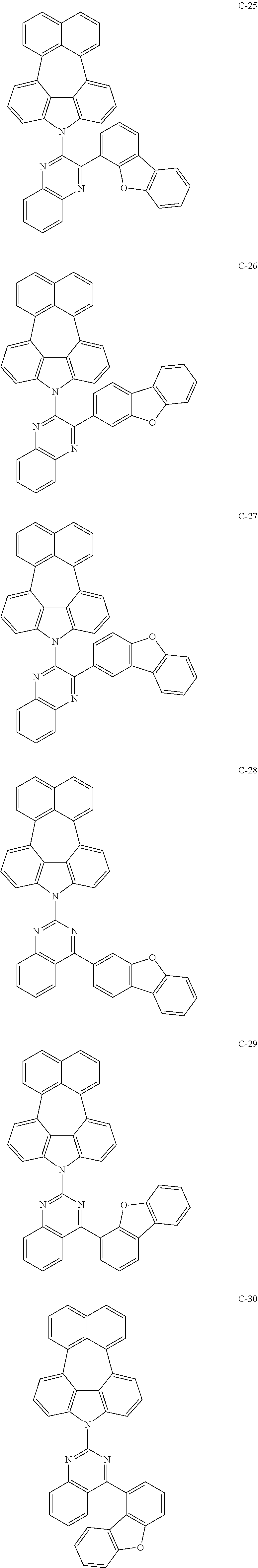

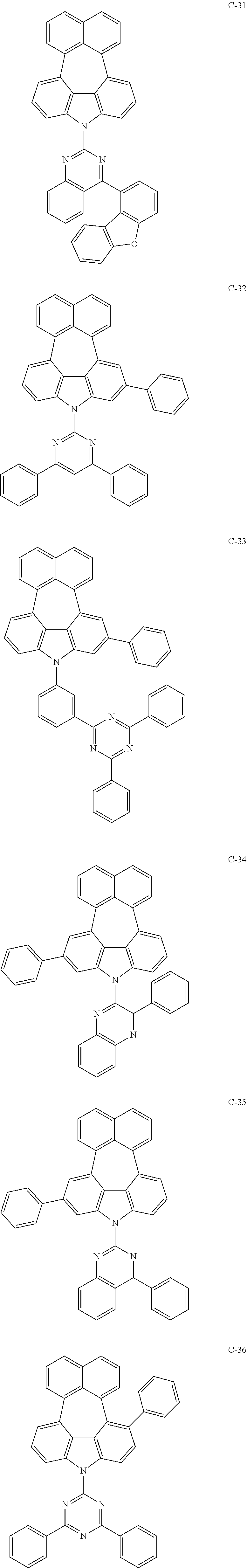

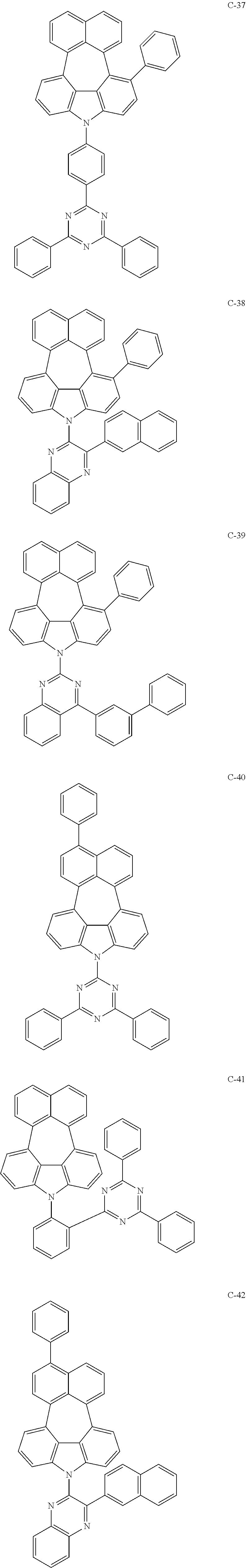

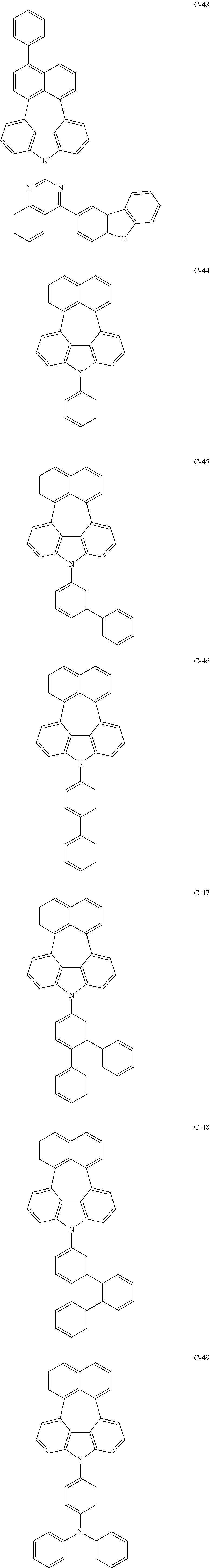

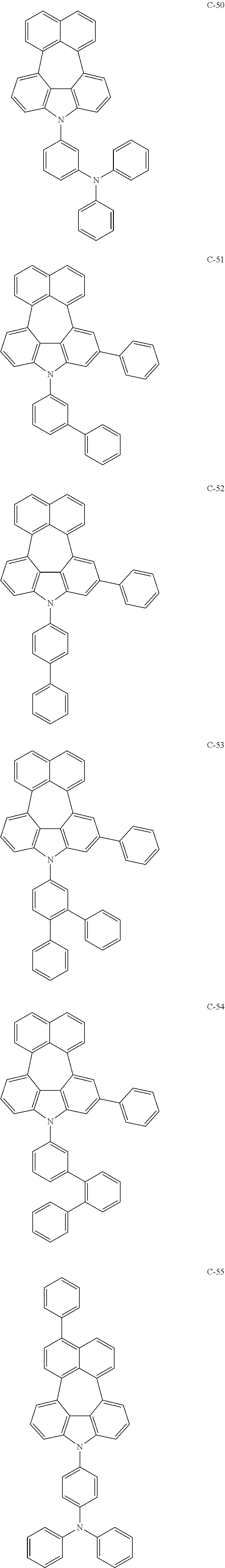 [SMILES: c1ccc(N(c2ccccc2)c2cccc(-n3c4cccc5c4c4c(cccc43)-c3cccc4cccc-5c34)c2)cc1 ; c1ccc(-c2cccc(-n3c4cccc5c4c4c(cc(-c6ccccc6)cc43)-c3cccc4cccc-5c34)c2)cc1 ; c1ccc(-c2ccc(-n3c4cccc5c4c4c(cc(-c6ccccc6)cc43)-c3cccc4cccc-5c34)cc2)cc1 ; c1ccc(-c2cc3c4c5c(cccc5n(-c5ccc(-c6ccccc6)c(-c6ccccc6)c5)c4c2)-c2cccc4cccc-3c24)cc1 ; c1ccc(-c2cc3c4c5c(cccc5n(-c5cccc(-c6ccccc6-c6ccccc6)c5)c4c2)-c2cccc4cccc-3c24)cc1 ; c1ccc(-c2ccc3c4c(cccc24)-c2cccc4c2c2c-3cccc2n4-c2ccc(N(c3ccccc3)c3ccccc3)cc2)cc1]

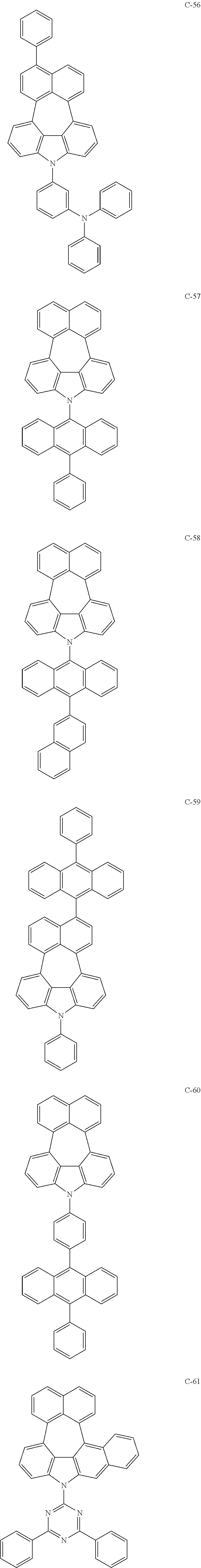 [SMILES: c1ccc(-c2ccc3c4c(cccc24)-c2cccc4c2c2c-3cccc2n4-c2cccc(N(c3ccccc3)c3ccccc3)c2)cc1 ; c1ccc(-c2c3ccccc3c(-n3c4cccc5c4c4c(cccc43)-c3cccc4cccc-5c34)c3ccccc23)cc1 ; c1ccc2cc(-c3c4ccccc4c(-n4c5cccc6c5c5c(cccc54)-c4cccc5cccc-6c45)c4ccccc34)ccc2c1 ; c1ccc(-c2c3ccccc3c(-c3ccc4c5c(cccc35)-c3cccc5c3c3c-4cccc3n5-c3ccccc3)c3ccccc23)cc1 ; c1ccc(-c2c3ccccc3c(-c3ccc(-n4c5cccc6c5c5c(cccc54)-c4cccc5cccc-6c45)cc3)c3ccccc23)cc1 ; c1ccc(-c2nc(-c3ccccc3)nc(-n3c4cccc5c4c4c(c6ccccc6cc43)-c3cccc4cccc-5c34)n2)cc1]

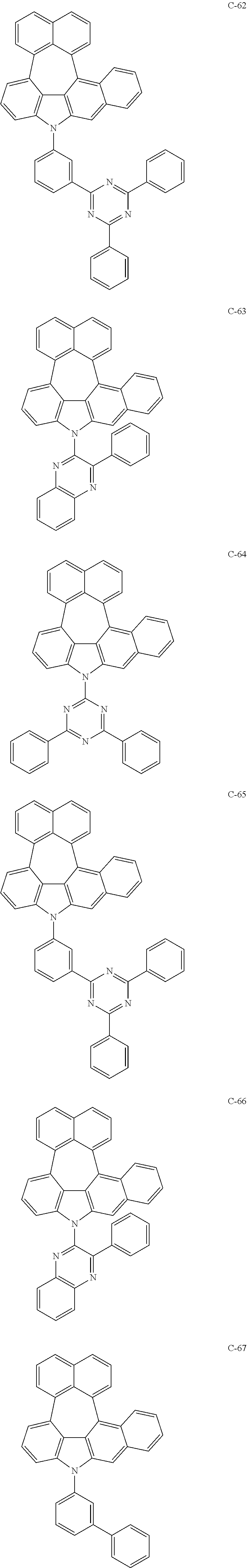

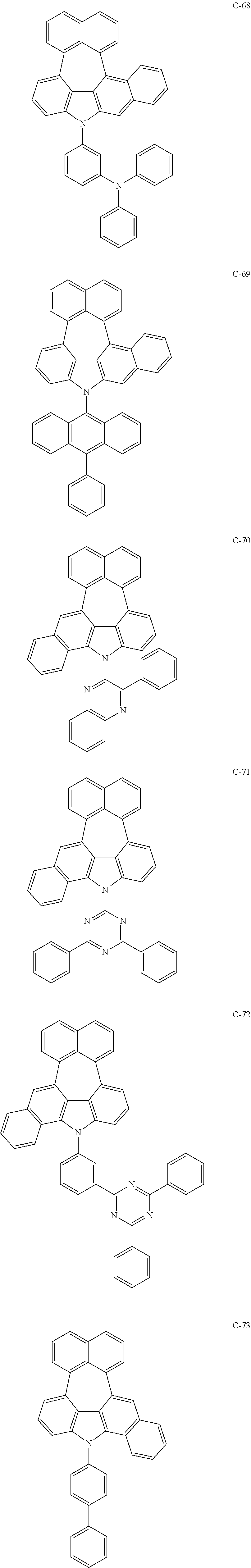

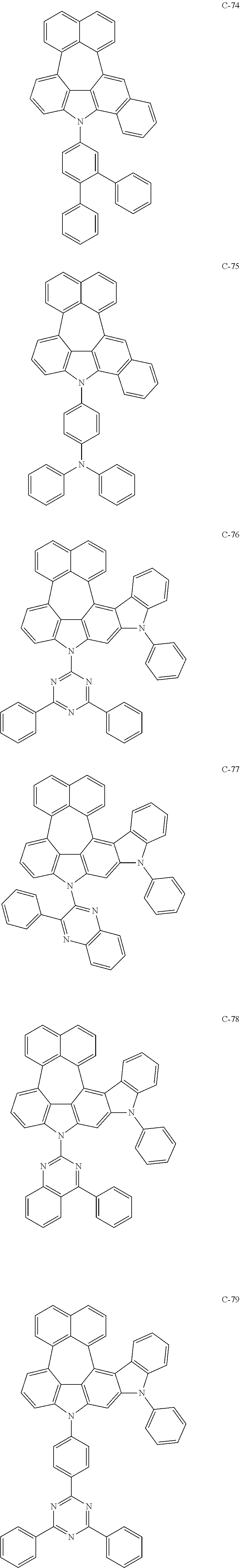

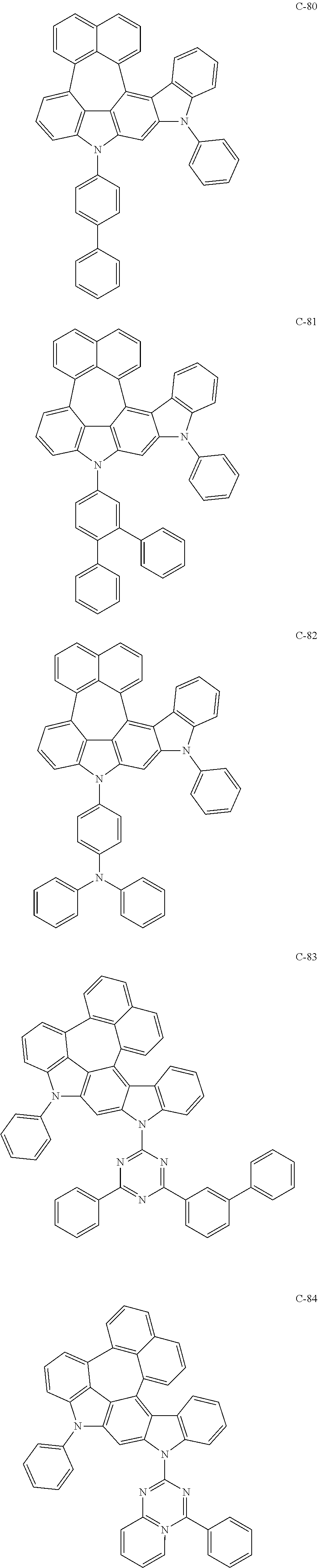

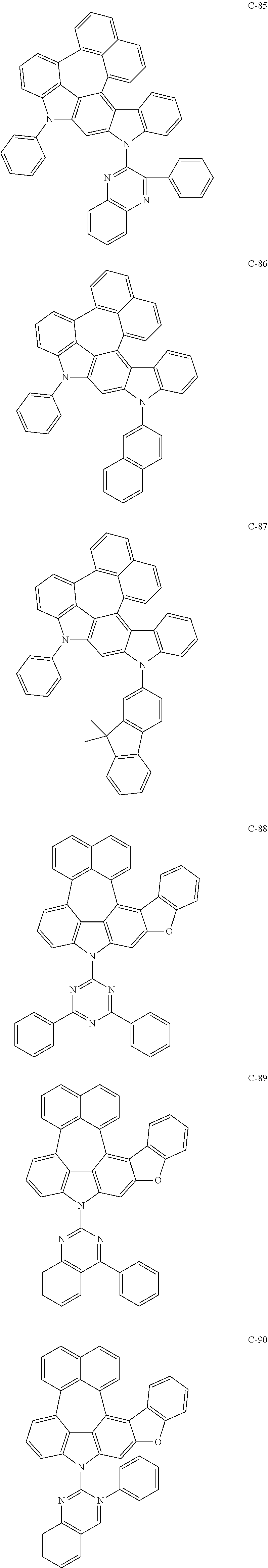

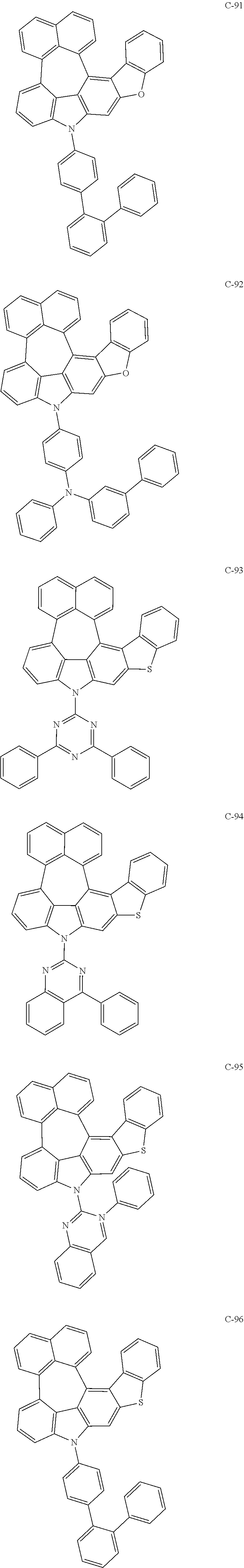

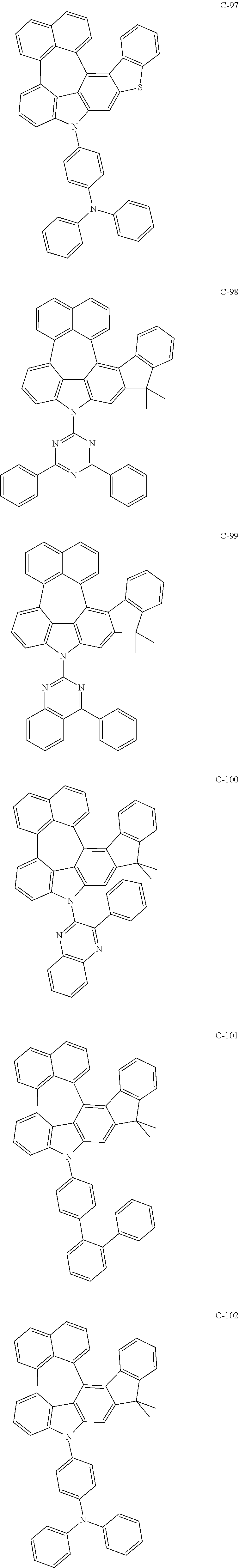

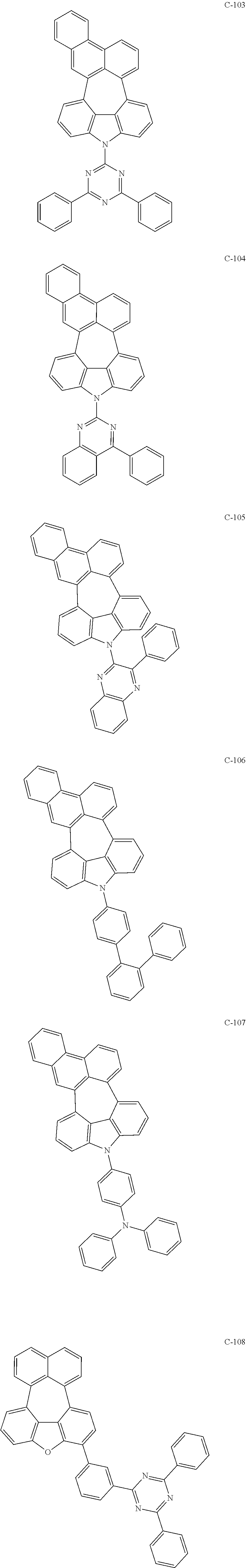 [SMILES: c1ccc(-c2nc(-c3ccccc3)nc(-n3c4cccc5c4c4c(cccc43)-c3cc4ccccc4c4cccc-5c34)n2)cc1 ; c1ccc(-c2nc(-n3c4cccc5c4c4c(cccc43)-c3cc4ccccc4c4cccc-5c34)nc3ccccc23)cc1 ; c1ccc(-c2nc3ccccc3nc2-n2c3cccc4c3c3c(cccc32)-c2cc3ccccc3c3cccc-4c23)cc1 ; c1ccc(-c2ccccc2-c2ccc(-n3c4cccc5c4c4c(cccc43)-c3cc4ccccc4c4cccc-5c34)cc2)cc1 ; c1ccc(N(c2ccccc2)c2ccc(-n3c4cccc5c4c4c(cccc43)-c3cc4ccccc4c4cccc-5c34)cc2)cc1 ; c1ccc(-c2nc(-c3ccccc3)nc(-c3cccc(-c4ccc5c6c4oc4cccc(c46)-c4cccc6cccc-5c46)c3)n2)cc1]

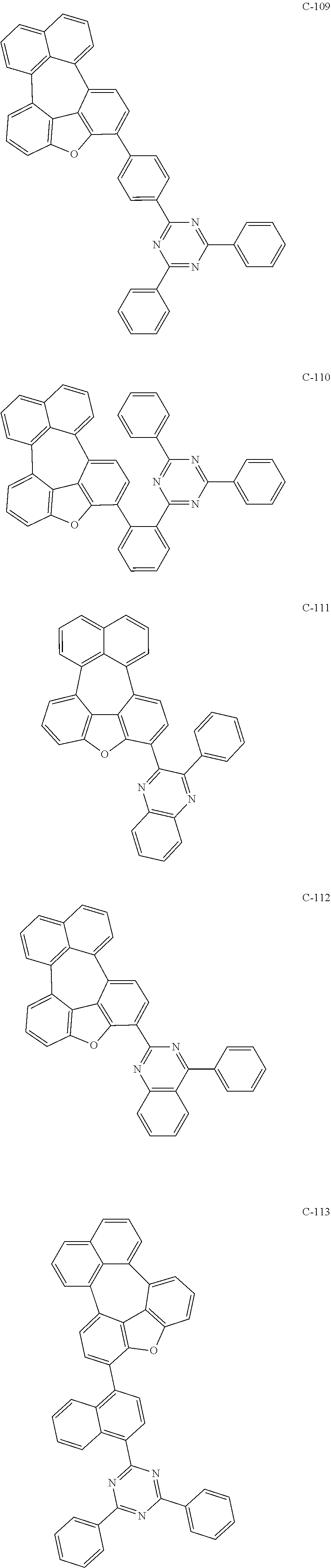 [SMILES: c1ccc(-c2nc(-c3ccccc3)nc(-c3ccc(-c4ccc5c6c4oc4cccc(c46)-c4cccc6cccc-5c46)cc3)n2)cc1 ; c1ccc(-c2nc(-c3ccccc3)nc(-c3ccccc3-c3ccc4c5c3oc3cccc(c35)-c3cccc5cccc-4c35)n2)cc1 ; c1ccc(-c2nc3ccccc3nc2-c2ccc3c4c2oc2cccc(c24)-c2cccc4cccc-3c24)cc1 ; c1ccc(-c2nc(-c3ccc4c5c3oc3cccc(c35)-c3cccc5cccc-4c35)nc3ccccc23)cc1 ; c1ccc(-c2nc(-c3ccccc3)nc(-c3ccc(-c4ccc5c6c4oc4cccc(c46)-c4cccc6cccc-5c46)c4ccccc34)n2)cc1]

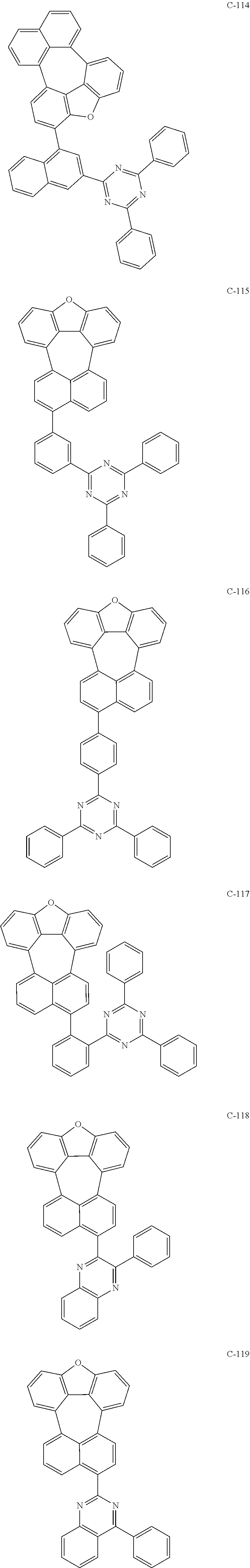 [SMILES: c1ccc(-c2nc(-c3ccccc3)nc(-c3cc(-c4ccc5c6c4oc4cccc(c46)-c4cccc6cccc-5c46)c4ccccc4c3)n2)cc1 ; c1ccc(-c2nc(-c3ccccc3)nc(-c3cccc(-c4ccc5c6c(cccc46)-c4cccc6oc7cccc-5c7c46)c3)n2)cc1 ; c1ccc(-c2nc(-c3ccccc3)nc(-c3ccc(-c4ccc5c6c(cccc46)-c4cccc6oc7cccc-5c7c46)cc3)n2)cc1 ; c1ccc(-c2nc(-c3ccccc3)nc(-c3ccccc3-c3ccc4c5c(cccc35)-c3cccc5oc6cccc-4c6c35)n2)cc1 ; c1ccc(-c2nc3ccccc3nc2-c2ccc3c4c(cccc24)-c2cccc4oc5cccc-3c5c24)cc1 ; c1ccc(-c2nc(-c3ccc4c5c(cccc35)-c3cccc5oc6cccc-4c6c35)nc3ccccc23)cc1]

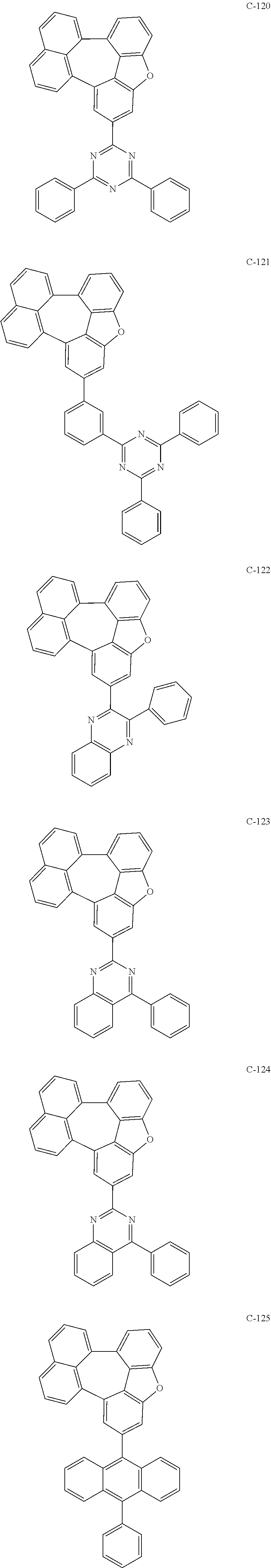 [SMILES: c1ccc(-c2nc(-c3ccccc3)nc(-c3cc4c5c(c3)oc3cccc(c35)-c3cccc5cccc-4c35)n2)cc1 ; c1ccc(-c2nc(-c3ccccc3)nc(-c3cccc(-c4cc5c6c(c4)oc4cccc(c46)-c4cccc6cccc-5c46)c3)n2)cc1 ; c1ccc(-c2nc3ccccc3nc2-c2cc3c4c(c2)oc2cccc(c24)-c2cccc4cccc-3c24)cc1 ; c1ccc(-c2nc(-c3cc4c5c(c3)oc3cccc(c35)-c3cccc5cccc-4c35)nc3ccccc23)cc1 ; c1ccc(-c2nc(-c3cc4c5c(c3)oc3cccc(c35)-c3cccc5cccc-4c35)nc3ccccc23)cc1 ; c1ccc(-c2c3ccccc3c(-c3cc4c5c(c3)oc3cccc(c35)-c3cccc5cccc-4c35)c3ccccc23)cc1]

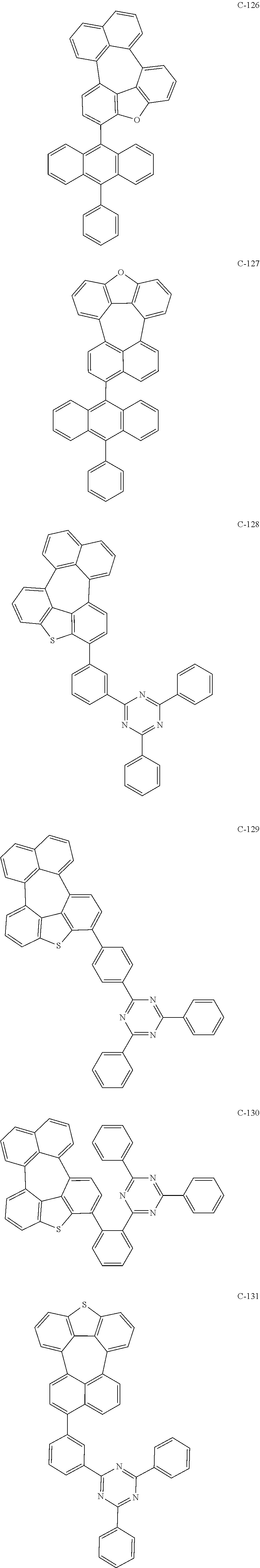 [SMILES: c1ccc(-c2c3ccccc3c(-c3ccc4c5c3oc3cccc(c35)-c3cccc5cccc-4c35)c3ccccc23)cc1 ; c1ccc(-c2c3ccccc3c(-c3ccc4c5c(cccc35)-c3cccc5oc6cccc-4c6c35)c3ccccc23)cc1 ; c1ccc(-c2nc(-c3ccccc3)nc(-c3cccc(-c4ccc5c6c4sc4cccc(c46)-c4cccc6cccc-5c46)c3)n2)cc1 ; c1ccc(-c2nc(-c3ccccc3)nc(-c3ccc(-c4ccc5c6c4sc4cccc(c46)-c4cccc6cccc-5c46)cc3)n2)cc1 ; c1ccc(-c2nc(-c3ccccc3)nc(-c3ccccc3-c3ccc4c5c3sc3cccc(c35)-c3cccc5cccc-4c35)n2)cc1 ; c1ccc(-c2nc(-c3ccccc3)nc(-c3cccc(-c4ccc5c6c(cccc46)-c4cccc6sc7cccc-5c7c46)c3)n2)cc1]

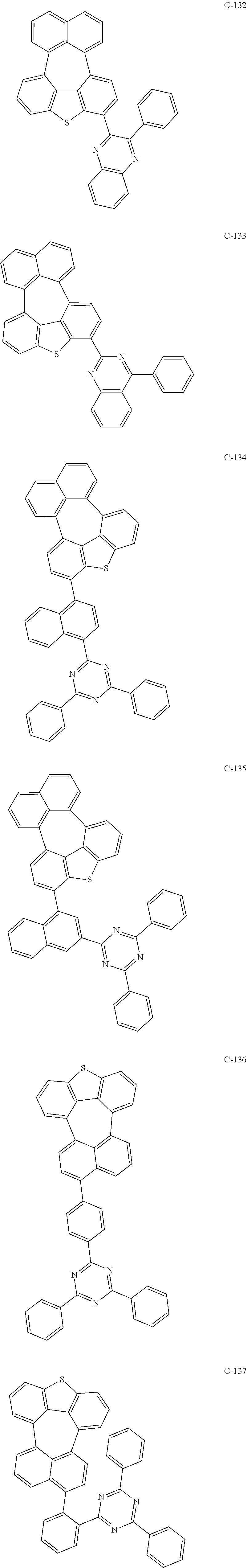 [SMILES: c1ccc(-c2nc3ccccc3nc2-c2ccc3c4c2sc2cccc(c24)-c2cccc4cccc-3c24)cc1 ; c1ccc(-c2nc(-c3ccc4c5c3sc3cccc(c35)-c3cccc5cccc-4c35)nc3ccccc23)cc1 ; c1ccc(-c2nc(-c3ccccc3)nc(-c3ccc(-c4ccc5c6c4sc4cccc(c46)-c4cccc6cccc-5c46)c4ccccc34)n2)cc1 ; c1ccc(-c2nc(-c3ccccc3)nc(-c3cc(-c4ccc5c6c4sc4cccc(c46)-c4cccc6cccc-5c46)c4ccccc4c3)n2)cc1 ; c1ccc(-c2nc(-c3ccccc3)nc(-c3ccc(-c4ccc5c6c(cccc46)-c4cccc6sc7cccc-5c7c46)cc3)n2)cc1 ; c1ccc(-c2nc(-c3ccccc3)nc(-c3ccccc3-c3ccc4c5c(cccc35)-c3cccc5sc6cccc-4c6c35)n2)cc1]

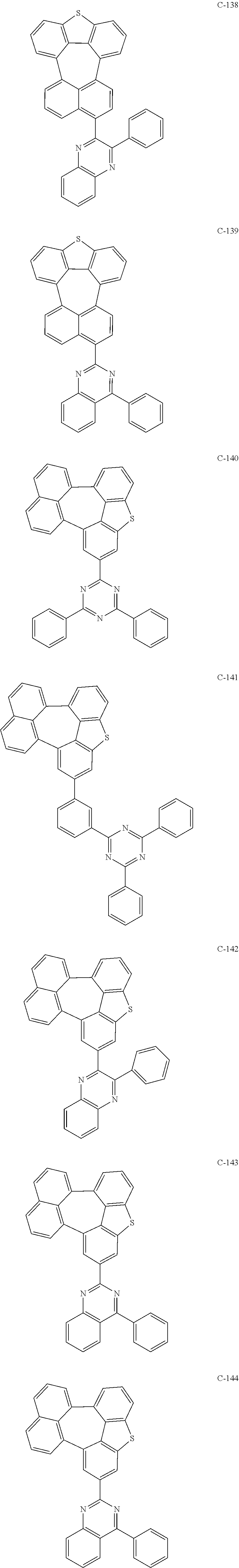

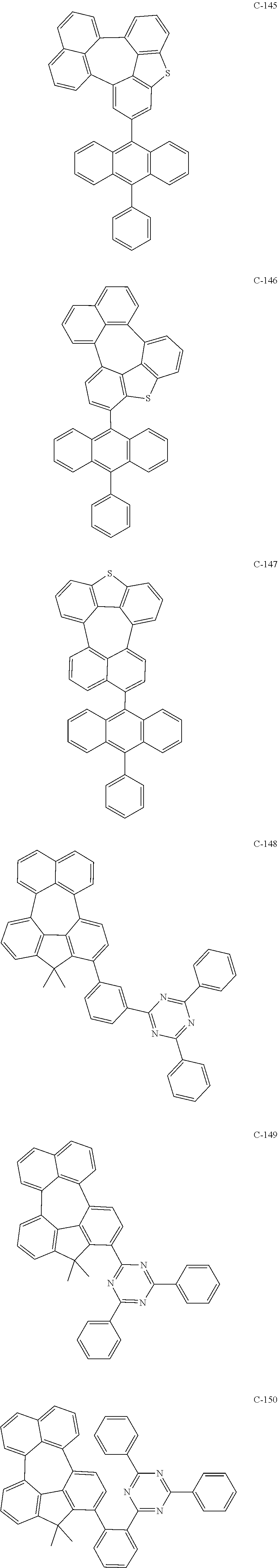 [SMILES: c1ccc(-c2c3ccccc3c(-c3cc4c5c(c3)sc3cccc(c35)-c3cccc5cccc-4c35)c3ccccc23)cc1 ; c1ccc(-c2c3ccccc3c(-c3ccc4c5c3sc3cccc(c35)-c3cccc5cccc-4c35)c3ccccc23)cc1 ; c1ccc(-c2c3ccccc3c(-c3ccc4c5c(cccc35)-c3cccc5sc6cccc-4c6c35)c3ccccc23)cc1 ; CC1(C)c2cccc3c2-c2c(ccc(-c4cccc(-c5nc(-c6ccccc6)nc(-c6ccccc6)n5)c4)c21)-c1cccc2cccc-3c12 ; CC1(C)c2cccc3c2-c2c(ccc(-c4nc(-c5ccccc5)nc(-c5ccccc5)n4)c21)-c1cccc2cccc-3c12 ; CC1(C)c2cccc3c2-c2c(ccc(-c4ccccc4-c4nc(-c5ccccc5)nc(-c5ccccc5)n4)c21)-c1cccc2cccc-3c12]

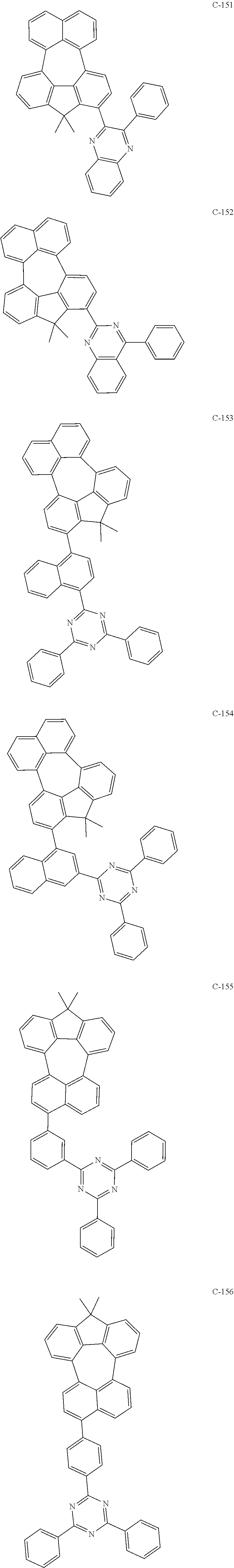

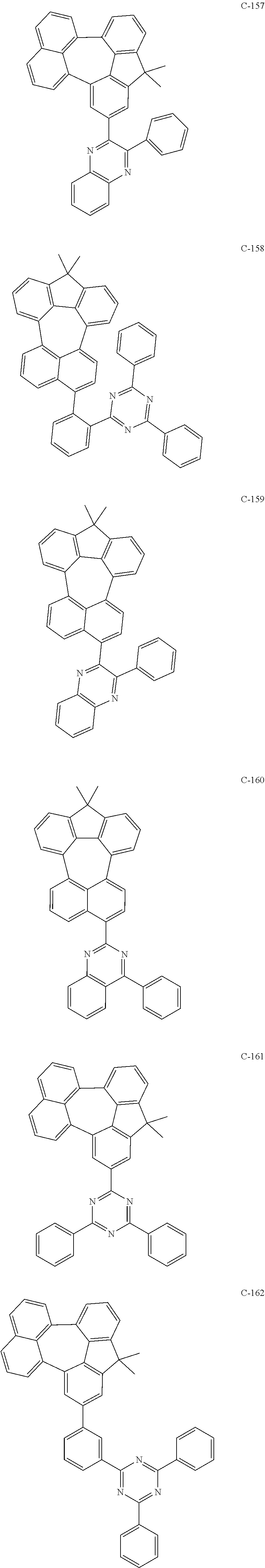 [SMILES: CC1(C)c2cccc3c2-c2c(cc(-c4nc5ccccc5nc4-c4ccccc4)cc21)-c1cccc2cccc-3c12 ; CC1(C)c2cccc3c2-c2c(cccc21)-c1ccc(-c2ccccc2-c2nc(-c4ccccc4)nc(-c4ccccc4)n2)c2cccc-3c12 ; CC1(C)c2cccc3c2-c2c(cccc21)-c1ccc(-c2nc4ccccc4nc2-c2ccccc2)c2cccc-3c12 ; CC1(C)c2cccc3c2-c2c(cccc21)-c1ccc(-c2nc(-c4ccccc4)c4ccccc4n2)c2cccc-3c12 ; CC1(C)c2cccc3c2-c2c(cc(-c4nc(-c5ccccc5)nc(-c5ccccc5)n4)cc21)-c1cccc2cccc-3c12 ; CC1(C)c2cccc3c2-c2c(cc(-c4cccc(-c5nc(-c6ccccc6)nc(-c6ccccc6)n5)c4)cc21)-c1cccc2cccc-3c12]

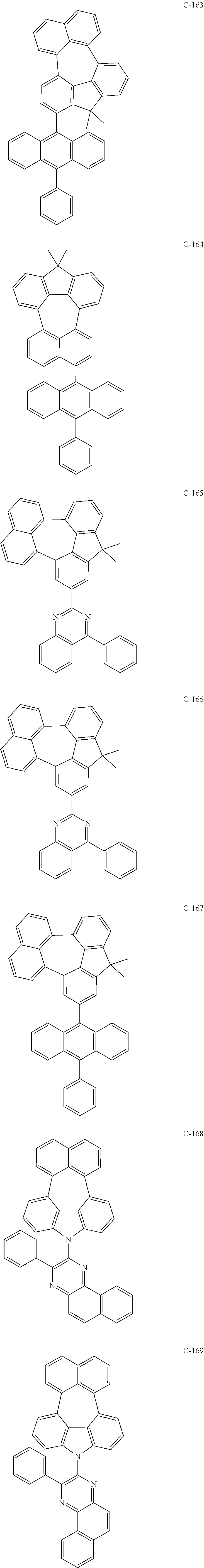

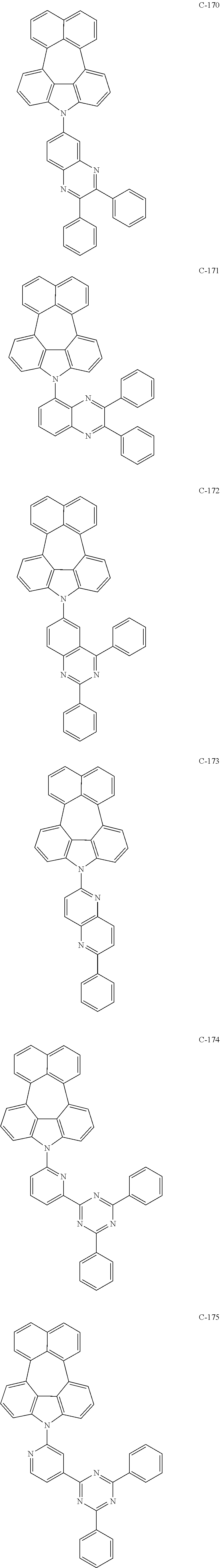

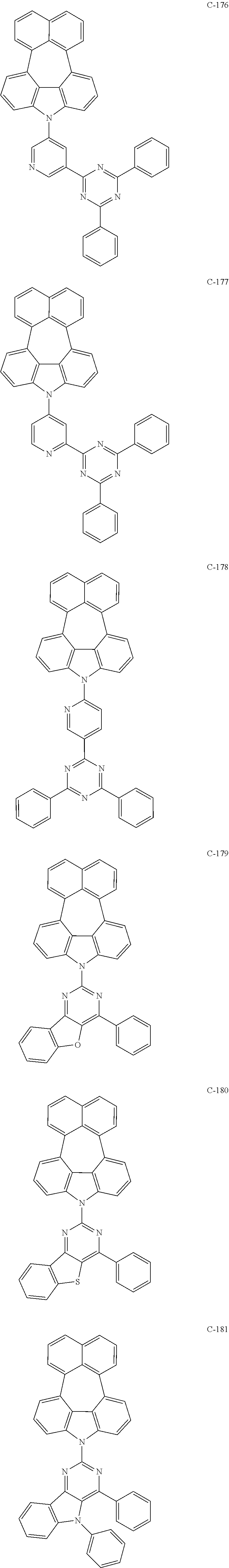 [SMILES: c1ccc(-c2nc(-c3ccccc3)nc(-c3cncc(-n4c5cccc6c5c5c(cccc54)-c4cccc5cccc-6c45)c3)n2)cc1 ; c1ccc(-c2nc(-c3ccccc3)nc(-c3cc(-n4c5cccc6c5c5c(cccc54)-c4cccc5cccc-6c45)ccn3)n2)cc1 ; c1ccc(-c2nc(-c3ccccc3)nc(-c3ccc(-n4c5cccc6c5c5c(cccc54)-c4cccc5cccc-6c45)nc3)n2)cc1 ; c1ccc(-c2nc(-n3c4cccc5c4c4c(cccc43)-c3cccc4cccc-5c34)nc3c2oc2ccccc23)cc1 ; c1ccc(-c2nc(-n3c4cccc5c4c4c(cccc43)-c3cccc4cccc-5c34)nc3c2sc2ccccc23)cc1 ; c1ccc(-c2nc(-n3c4cccc5c4c4c(cccc43)-c3cccc4cccc-5c34)nc3c4ccccc4n(-c4ccccc4)c23)cc1]

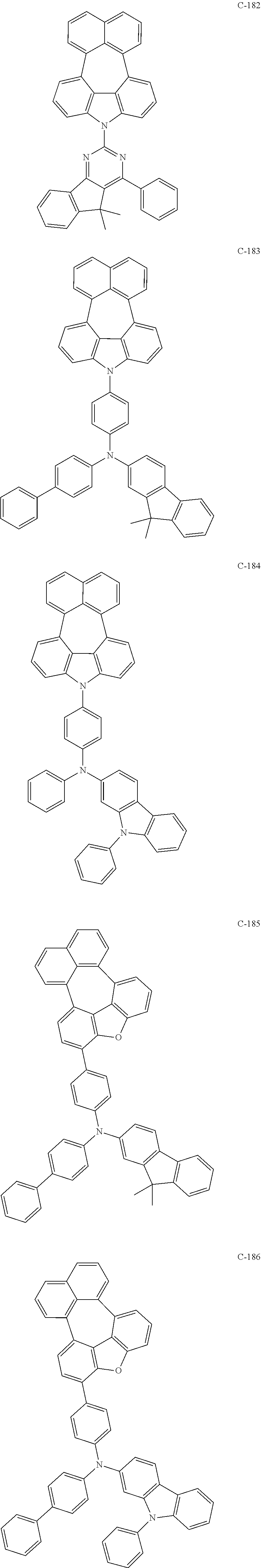 [SMILES: CC1(C)c2ccccc2-c2nc(-n3c4cccc5c4c4c(cccc43)-c3cccc4cccc-5c34)nc(-c3ccccc3)c21 ; CC1(C)c2ccccc2-c2ccc(N(c3ccc(-c4ccccc4)cc3)c3ccc(-n4c5cccc6c5c5c(cccc54)-c4cccc5cccc-6c45)cc3)cc21 ; c1ccc(N(c2ccc(-n3c4cccc5c4c4c(cccc43)-c3cccc4cccc-5c34)cc2)c2ccc3c4ccccc4n(-c4ccccc4)c3c2)cc1 ; CC1(C)c2ccccc2-c2ccc(N(c3ccc(-c4ccccc4)cc3)c3ccc(-c4ccc5c6c4oc4cccc(c46)-c4cccc6cccc-5c46)cc3)cc21 ; c1ccc(-c2ccc(N(c3ccc(-c4ccc5c6c4oc4cccc(c46)-c4cccc6cccc-5c46)cc3)c3ccc4c5ccccc5n(-c5ccccc5)c4c3)cc2)cc1]

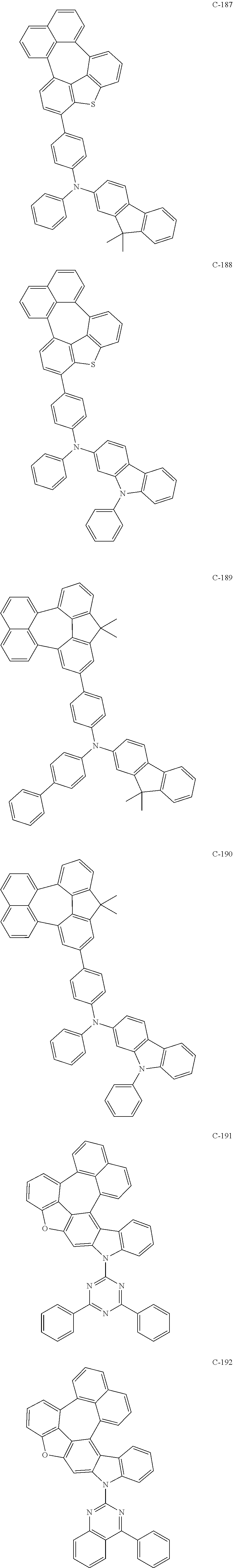

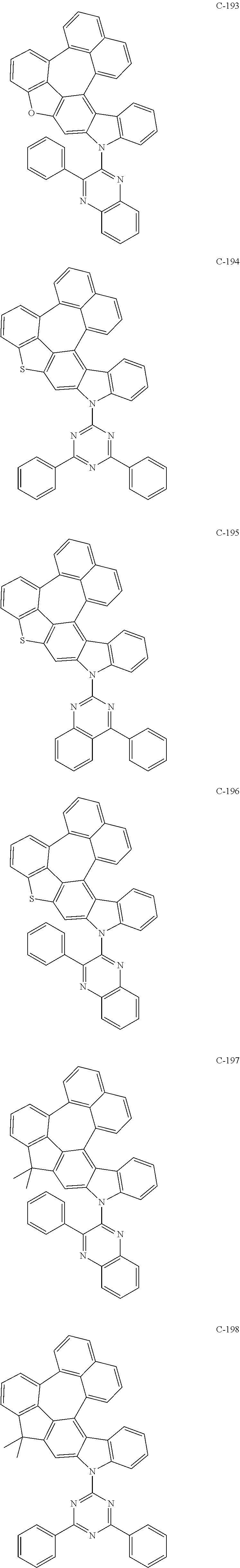

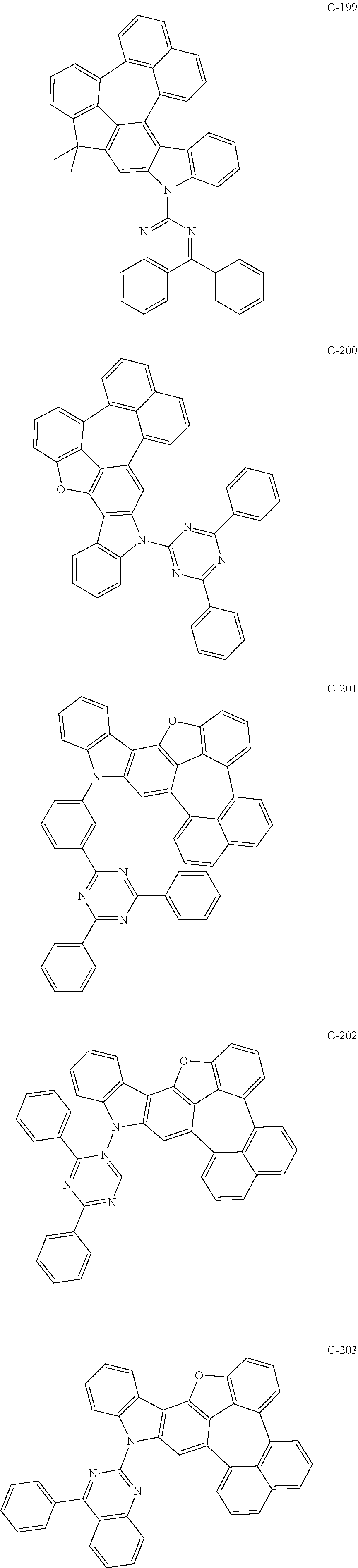

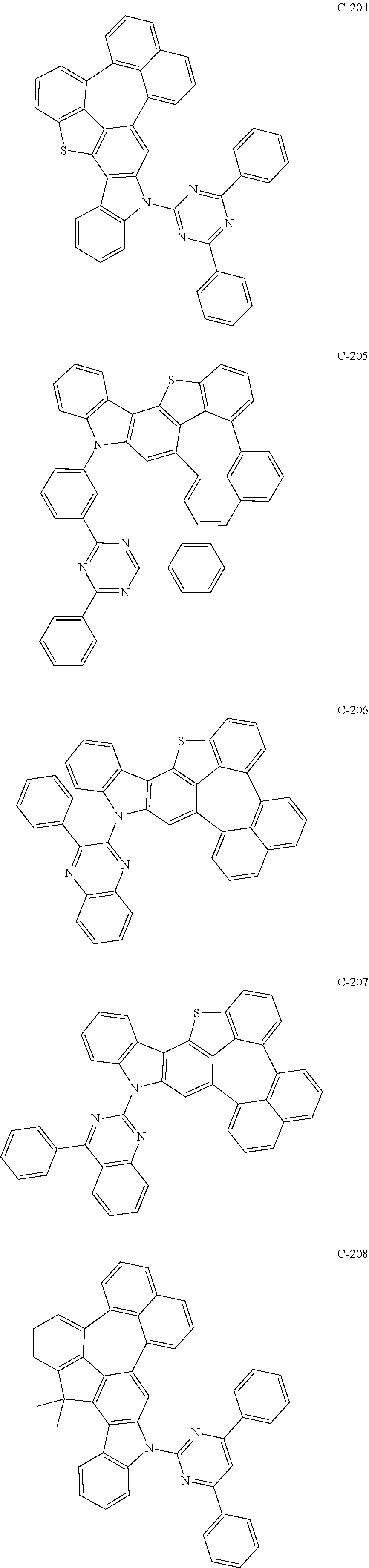 [SMILES: c1ccc(-c2nc(-c3ccccc3)nc(-n3c4ccccc4c4c5sc6cccc7c6c5c(cc43)-c3cccc4cccc-7c34)n2)cc1 ; c1ccc(-c2nc(-c3ccccc3)nc(-c3cccc(-n4c5ccccc5c5c6sc7cccc8c7c6c(cc54)-c4cccc5cccc-8c45)c3)n2)cc1 ; c1ccc(-c2nc3ccccc3nc2-n2c3ccccc3c3c4sc5cccc6c5c4c(cc32)-c2cccc3cccc-6c23)cc1 ; c1ccc(-c2nc(-n3c4ccccc4c4c5sc6cccc7c6c5c(cc43)-c3cccc4cccc-7c34)nc3ccccc23)cc1 ; CC1(C)c2cccc3c2-c2c(cc4c(c21)c1ccccc1n4-c1nc(-c2ccccc2)cc(-c2ccccc2)n1)-c1cccc2cccc-3c12]

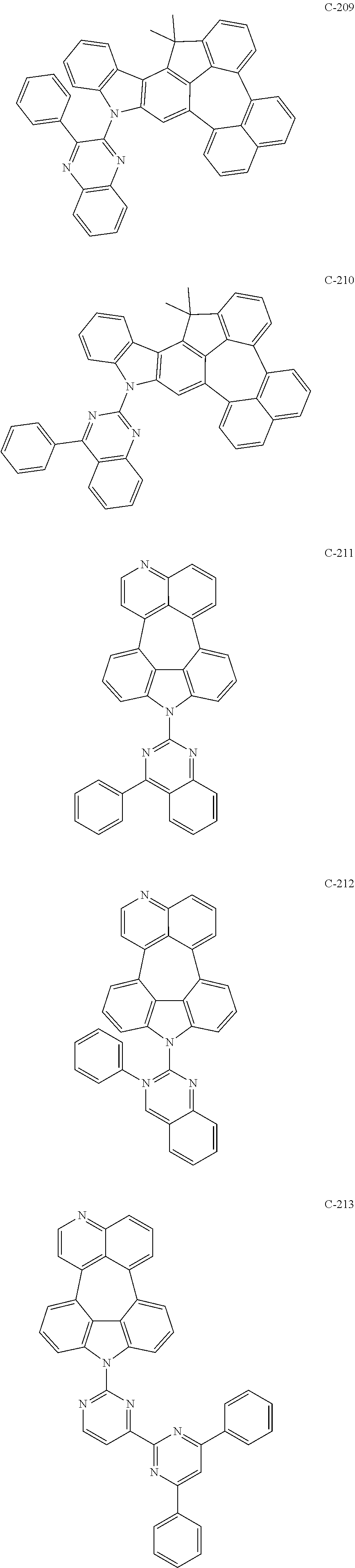

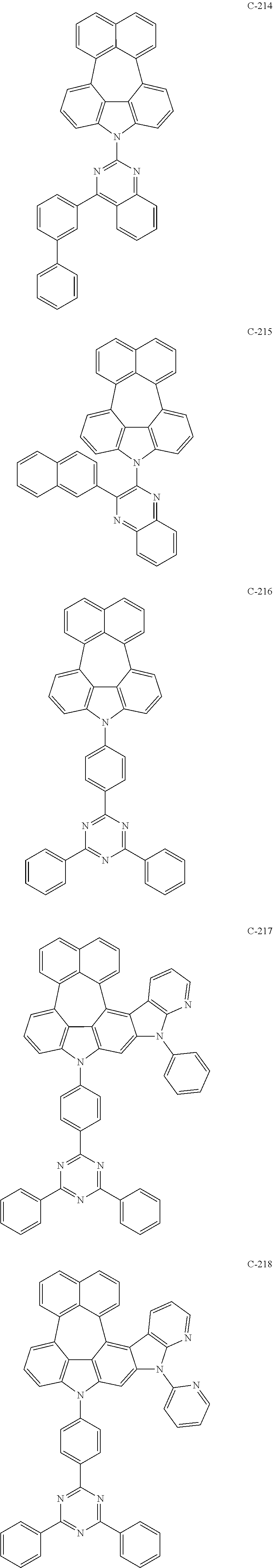 [SMILES: c1ccc(-c2cccc(-c3nc(-n4c5cccc6c5c5c(cccc54)-c4cccc5cccc-6c45)nc4ccccc34)c2)cc1 ; c1ccc2cc(-c3nc4ccccc4nc3-n3c4cccc5c4c4c(cccc43)-c3cccc4cccc-5c34)ccc2c1 ; c1ccc(-c2nc(-c3ccccc3)nc(-c3ccc(-n4c5cccc6c5c5c(cccc54)-c4cccc5cccc-6c45)cc3)n2)cc1 ; c1ccc(-c2nc(-c3ccccc3)nc(-c3ccc(-n4c5cccc6c5c5c(c7c8cccnc8n(-c8ccccc8)c7cc54)-c4cccc5cccc-6c45)cc3)n2)cc1 ; c1ccc(-c2nc(-c3ccccc3)nc(-c3ccc(-n4c5cccc6c5c5c(c7c8cccnc8n(-c8ccccn8)c7cc54)-c4cccc5cccc-6c45)cc3)n2)cc1]

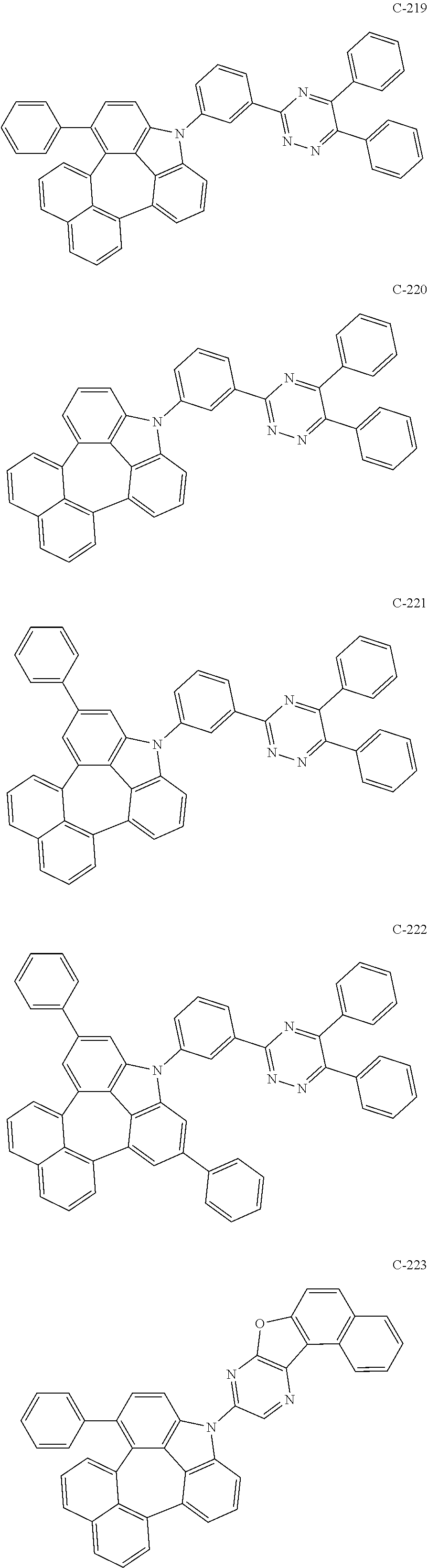 [SMILES: c1ccc(-c2ccc3c4c2-c2cccc5cccc(c25)-c2cccc(c24)n3-c2cccc(-c3nnc(-c4ccccc4)c(-c4ccccc4)n3)c2)cc1 ; c1ccc(-c2nnc(-c3cccc(-n4c5cccc6c5c5c(cccc54)-c4cccc5cccc-6c45)c3)nc2-c2ccccc2)cc1 ; c1ccc(-c2cc3c4c5c(cccc5n(-c5cccc(-c6nnc(-c7ccccc7)c(-c7ccccc7)n6)c5)c4c2)-c2cccc4cccc-3c24)cc1 ; c1ccc(-c2cc3c4c5c(cc(-c6ccccc6)cc5n(-c5cccc(-c6nnc(-c7ccccc7)c(-c7ccccc7)n6)c5)c4c2)-c2cccc4cccc-3c24)cc1 ; c1ccc(-c2ccc3c4c2-c2cccc5cccc(c25)-c2cccc(c24)n3-c2cnc3c(n2)oc2ccc4ccccc4c23)cc1]

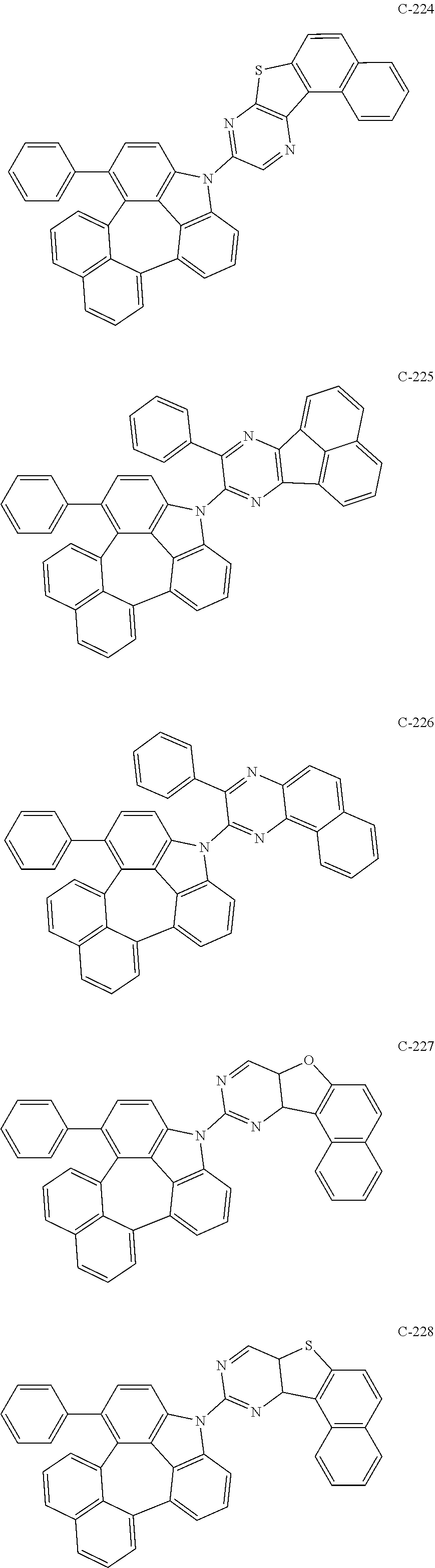 [SMILES: c1ccc(-c2ccc3c4c2-c2cccc5cccc(c25)-c2cccc(c24)n3-c2cnc3c(n2)sc2ccc4ccccc4c23)cc1 ; c1ccc(-c2ccc3c4c2-c2cccc5cccc(c25)-c2cccc(c24)n3-c2nc3c(nc2-c2ccccc2)-c2cccc4cccc-3c24)cc1 ; c1ccc(-c2ccc3c4c2-c2cccc5cccc(c25)-c2cccc(c24)n3-c2nc3c(ccc4ccccc43)nc2-c2ccccc2)cc1 ; C1=NC(n2c3cccc4c3c3c(c(-c5ccccc5)ccc32)-c2cccc3cccc-4c23)=NC2c3c(ccc4ccccc34)OC12 ; C1=NC(n2c3cccc4c3c3c(c(-c5ccccc5)ccc32)-c2cccc3cccc-4c23)=NC2c3c(ccc4ccccc34)SC12]

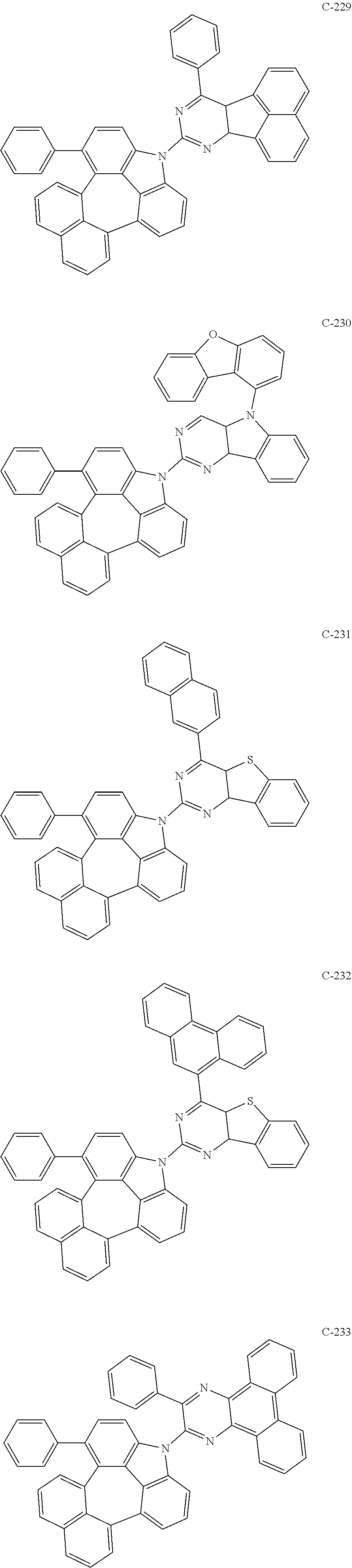 [SMILES: c1ccc(C2=NC(n3c4cccc5c4c4c(c(-c6ccccc6)ccc43)-c3cccc4cccc-5c34)=NC3c4cccc5cccc(c45)C23)cc1 ; C1=NC(n2c3cccc4c3c3c(c(-c5ccccc5)ccc32)-c2cccc3cccc-4c23)=NC2c3ccccc3N(c3cccc4oc5ccccc5c34)C12 ; c1ccc(-c2ccc3c4c2-c2cccc5cccc(c25)-c2cccc(c24)n3C2=NC3c4ccccc4SC3C(c3ccc4ccccc4c3)=N2)cc1 ; c1ccc(-c2ccc3c4c2-c2cccc5cccc(c25)-c2cccc(c24)n3C2=NC3c4ccccc4SC3C(c3cc4ccccc4c4ccccc34)=N2)cc1 ; c1ccc(-c2ccc3c4c2-c2cccc5cccc(c25)-c2cccc(c24)n3-c2nc3c4ccccc4c4ccccc4c3nc2-c2ccccc2)cc1]

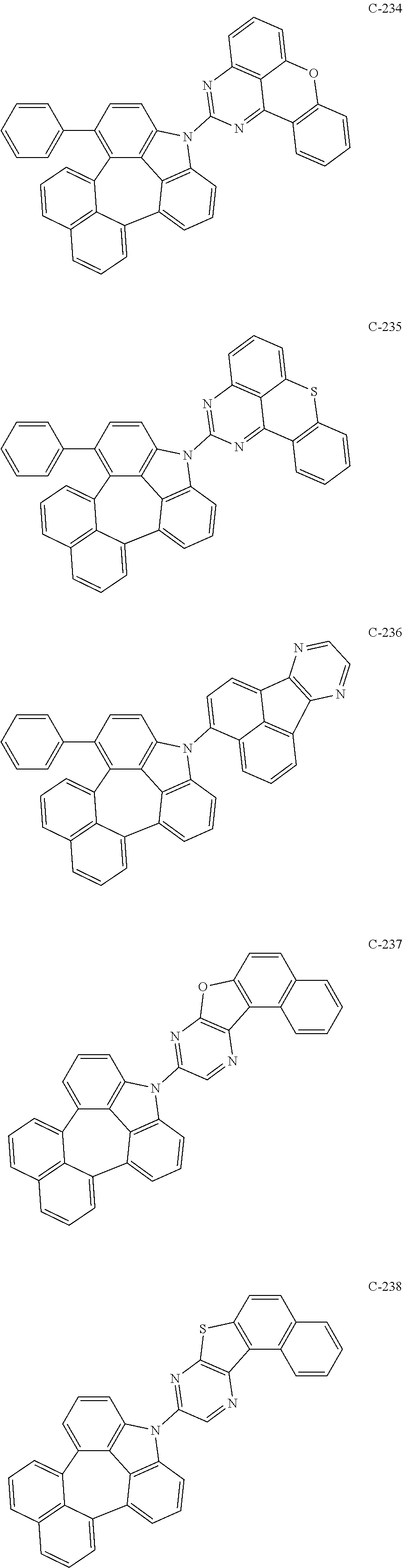 [SMILES: c1ccc(-c2ccc3c4c2-c2cccc5cccc(c25)-c2cccc(c24)n3-c2nc3c4c(cccc4n2)Oc2ccccc2-3)cc1 ; c1ccc(-c2ccc3c4c2-c2cccc5cccc(c25)-c2cccc(c24)n3-c2nc3c4c(cccc4n2)Sc2ccccc2-3)cc1 ; c1ccc(-c2ccc3c4c2-c2cccc5cccc(c25)-c2cccc(c24)n3-c2ccc3c4c(cccc24)-c2nccnc2-3)cc1 ; c1cc2c3c(cccc3c1)-c1cccc3c1c1c-2cccc1n3-c1cnc2c(n1)oc1ccc3ccccc3c12 ; c1cc2c3c(cccc3c1)-c1cccc3c1c1c-2cccc1n3-c1cnc2c(n1)sc1ccc3ccccc3c12]

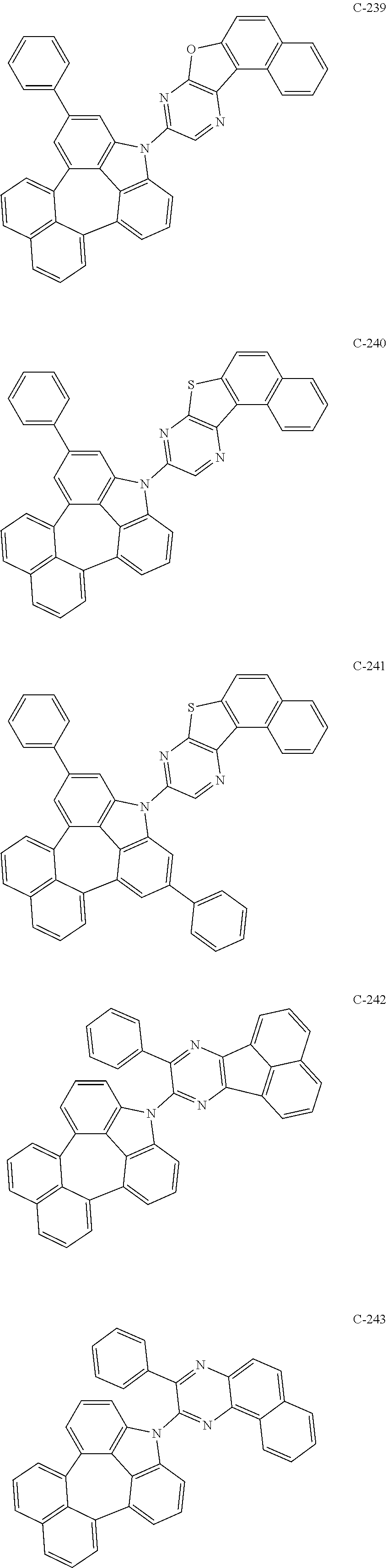 [SMILES: c1ccc(-c2cc3c4c5c(cccc5n(-c5cnc6c(n5)oc5ccc7ccccc7c56)c4c2)-c2cccc4cccc-3c24)cc1 ; c1ccc(-c2cc3c4c5c(cccc5n(-c5cnc6c(n5)sc5ccc7ccccc7c56)c4c2)-c2cccc4cccc-3c24)cc1 ; c1ccc(-c2cc3c4c5c(cc(-c6ccccc6)cc5n(-c5cnc6c(n5)sc5ccc7ccccc7c56)c4c2)-c2cccc4cccc-3c24)cc1 ; c1ccc(-c2nc3c(nc2-n2c4cccc5c4c4c(cccc42)-c2cccc4cccc-5c24)-c2cccc4cccc-3c24)cc1 ; c1ccc(-c2nc3ccc4ccccc4c3nc2-n2c3cccc4c3c3c(cccc32)-c2cccc3cccc-4c23)cc1]

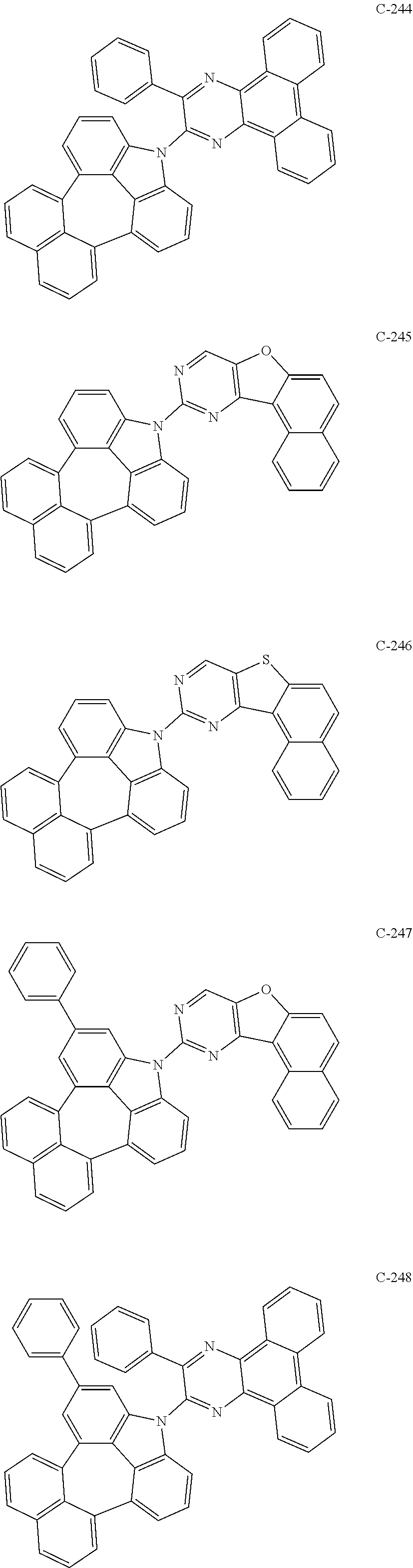 [SMILES: c1ccc(-c2nc3c4ccccc4c4ccccc4c3nc2-n2c3cccc4c3c3c(cccc32)-c2cccc3cccc-4c23)cc1 ; c1cc2c3c(cccc3c1)-c1cccc3c1c1c-2cccc1n3-c1ncc2oc3ccc4ccccc4c3c2n1 ; c1cc2c3c(cccc3c1)-c1cccc3c1c1c-2cccc1n3-c1ncc2sc3ccc4ccccc4c3c2n1 ; c1ccc(-c2cc3c4c5c(cccc5n(-c5ncc6oc7ccc8ccccc8c7c6n5)c4c2)-c2cccc4cccc-3c24)cc1 ; c1ccc(-c2cc3c4c5c(cccc5n(-c5nc6c7ccccc7c7ccccc7c6nc5-c5ccccc5)c4c2)-c2cccc4cccc-3c24)cc1]

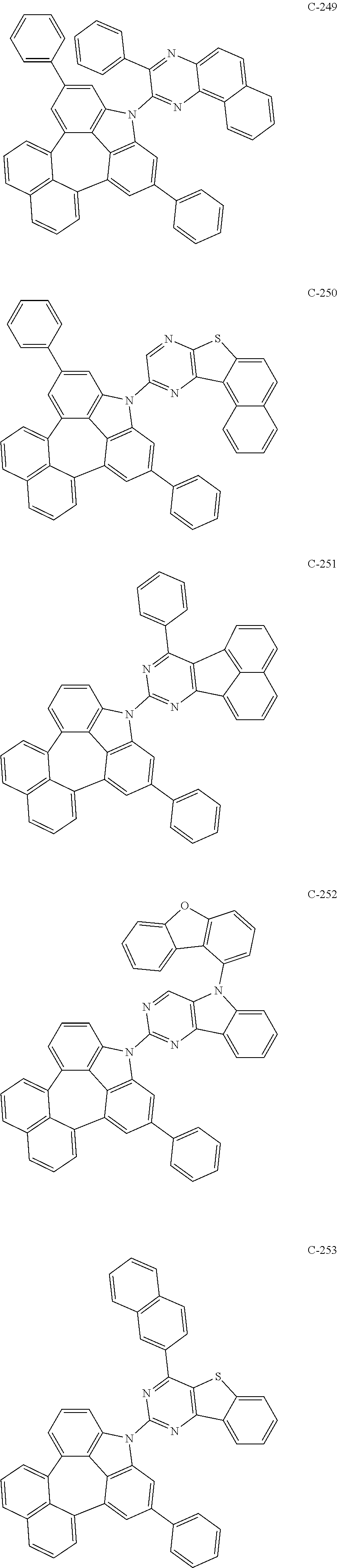 [SMILES: c1ccc(-c2cc3c4c5c(cc(-c6ccccc6)cc5n(-c5nc6c(ccc7ccccc76)nc5-c5ccccc5)c4c2)-c2cccc4cccc-3c24)cc1 ; c1ccc(-c2cc3c4c5c(cc(-c6ccccc6)cc5n(-c5cnc6sc7ccc8ccccc8c7c6n5)c4c2)-c2cccc4cccc-3c24)cc1 ; c1ccc(-c2cc3c4c5c(cccc5n(-c5nc(-c6ccccc6)c6c(n5)-c5cccc7cccc-6c57)c4c2)-c2cccc4cccc-3c24)cc1 ; c1ccc(-c2cc3c4c5c(cccc5n(-c5ncc6c(n5)c5ccccc5n6-c5cccc6oc7ccccc7c56)c4c2)-c2cccc4cccc-3c24)cc1 ; c1ccc(-c2cc3c4c5c(cccc5n(-c5nc(-c6ccc7ccccc7c6)c6sc7ccccc7c6n5)c4c2)-c2cccc4cccc-3c24)cc1]

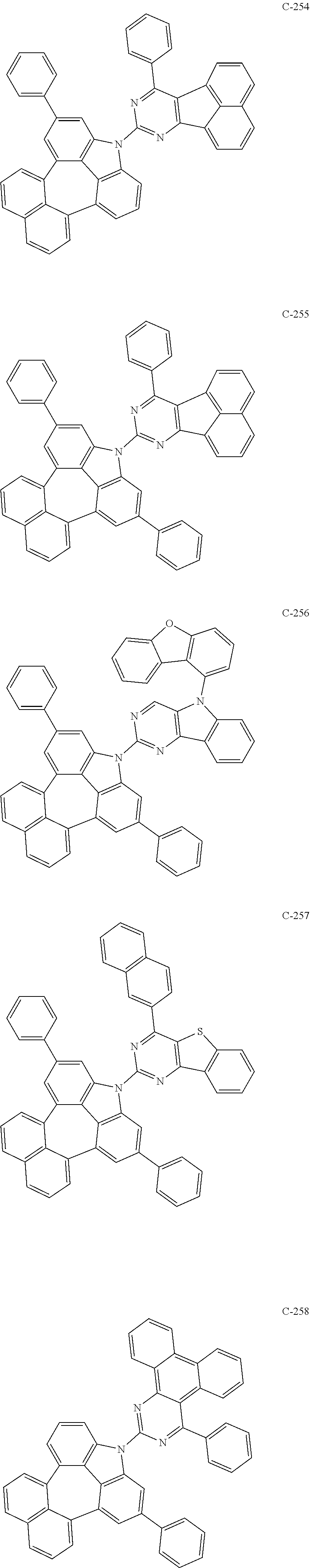

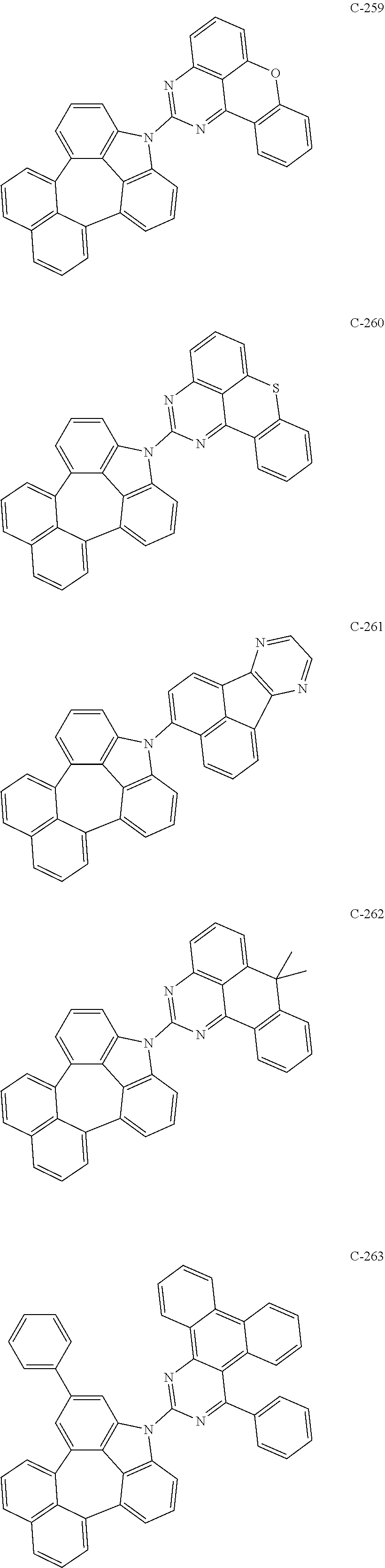 [SMILES: c1ccc2c(c1)Oc1cccc3nc(-n4c5cccc6c5c5c(cccc54)-c4cccc5cccc-6c45)nc-2c13 ; c1ccc2c(c1)Sc1cccc3nc(-n4c5cccc6c5c5c(cccc54)-c4cccc5cccc-6c45)nc-2c13 ; c1cc2c3c(cccc3c1)-c1cccc3c1c1c-2cccc1n3-c1ccc2c3c(cccc13)-c1nccnc1-2 ; CC1(C)c2ccccc2-c2nc(-n3c4cccc5c4c4c(cccc43)-c3cccc4cccc-5c34)nc3cccc1c23 ; c1ccc(-c2cc3c4c5c(cccc5n(-c5nc(-c6ccccc6)c6c7ccccc7c7ccccc7c6n5)c4c2)-c2cccc4cccc-3c24)cc1]

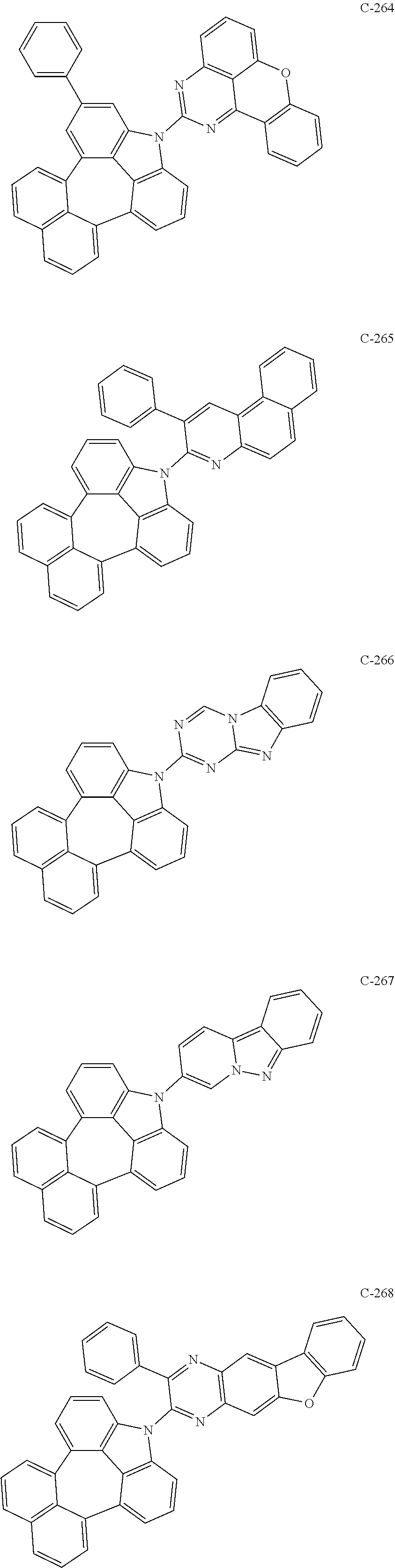 [SMILES: c1ccc(-c2cc3c4c5c(cccc5n(-c5nc6c7c(cccc7n5)Oc5ccccc5-6)c4c2)-c2cccc4cccc-3c24)cc1 ; c1ccc(-c2cc3c(ccc4ccccc43)nc2-n2c3cccc4c3c3c(cccc32)-c2cccc3cccc-4c23)cc1 ; c1cc2c3c(cccc3c1)-c1cccc3c1c1c-2cccc1n3-c1ncn2c(n1)nc1ccccc12 ; c1cc2c3c(cccc3c1)-c1cccc3c1c1c-2cccc1n3-c1ccc2c3ccccc3nn2c1 ; c1ccc(-c2nc3cc4c(cc3nc2-n2c3cccc5c3c3c(cccc32)-c2cccc3cccc-5c23)oc2ccccc24)cc1]

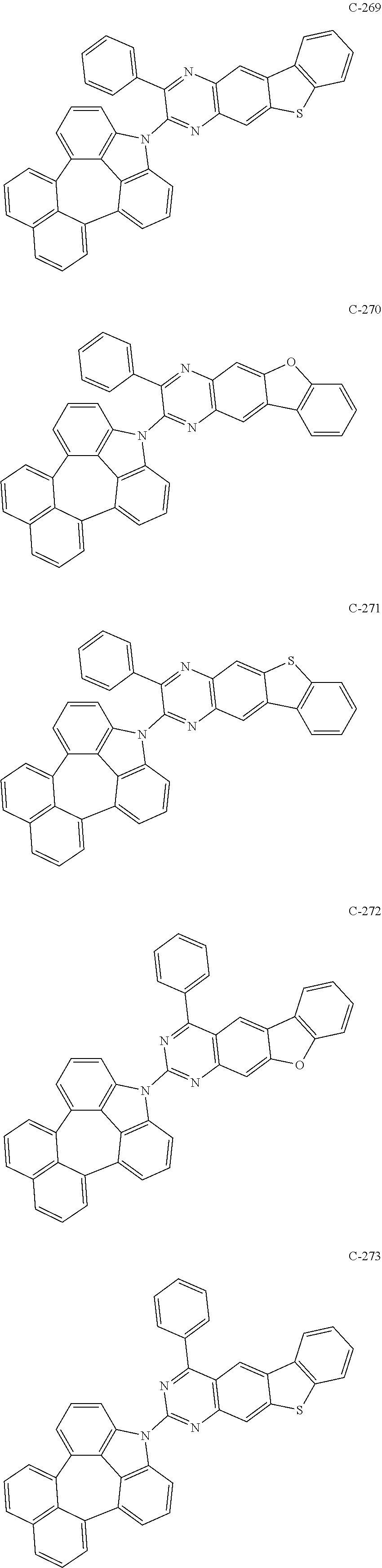 [SMILES: c1ccc(-c2nc3cc4c(cc3nc2-n2c3cccc5c3c3c(cccc32)-c2cccc3cccc-5c23)sc2ccccc24)cc1 ; c1ccc(-c2nc3cc4oc5ccccc5c4cc3nc2-n2c3cccc4c3c3c(cccc32)-c2cccc3cccc-4c23)cc1 ; c1ccc(-c2nc3cc4sc5ccccc5c4cc3nc2-n2c3cccc4c3c3c(cccc32)-c2cccc3cccc-4c23)cc1 ; c1ccc(-c2nc(-n3c4cccc5c4c4c(cccc43)-c3cccc4cccc-5c34)nc3cc4oc5ccccc5c4cc23)cc1 ; c1ccc(-c2nc(-n3c4cccc5c4c4c(cccc43)-c3cccc4cccc-5c34)nc3cc4sc5ccccc5c4cc23)cc1]

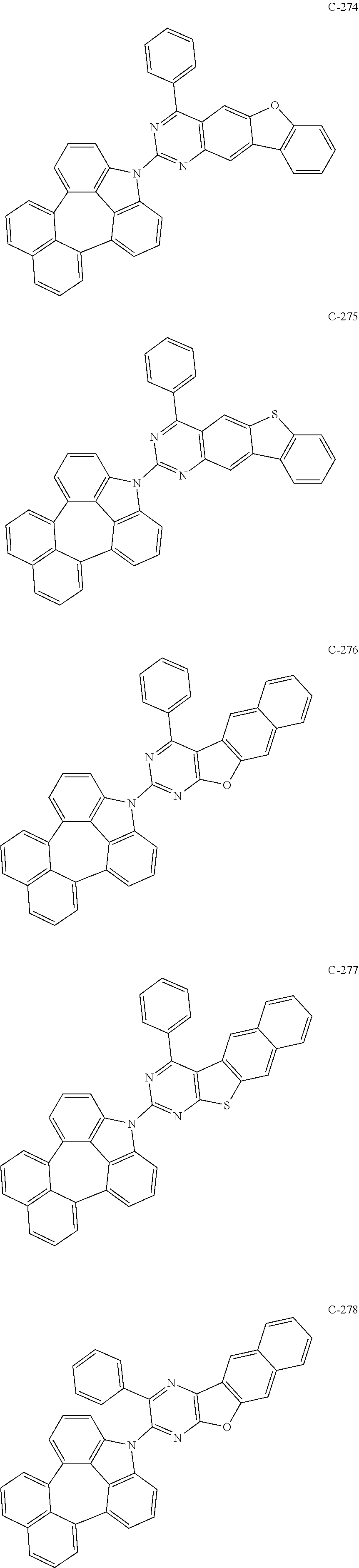 [SMILES: c1ccc(-c2nc(-n3c4cccc5c4c4c(cccc43)-c3cccc4cccc-5c34)nc3cc4c(cc23)oc2ccccc24)cc1 ; c1ccc(-c2nc(-n3c4cccc5c4c4c(cccc43)-c3cccc4cccc-5c34)nc3cc4c(cc23)sc2ccccc24)cc1 ; c1ccc(-c2nc(-n3c4cccc5c4c4c(cccc43)-c3cccc4cccc-5c34)nc3oc4cc5ccccc5cc4c23)cc1 ; c1ccc(-c2nc(-n3c4cccc5c4c4c(cccc43)-c3cccc4cccc-5c34)nc3sc4cc5ccccc5cc4c23)cc1 ; c1ccc(-c2nc3c(nc2-n2c4cccc5c4c4c(cccc42)-c2cccc4cccc-5c24)oc2cc4ccccc4cc23)cc1]

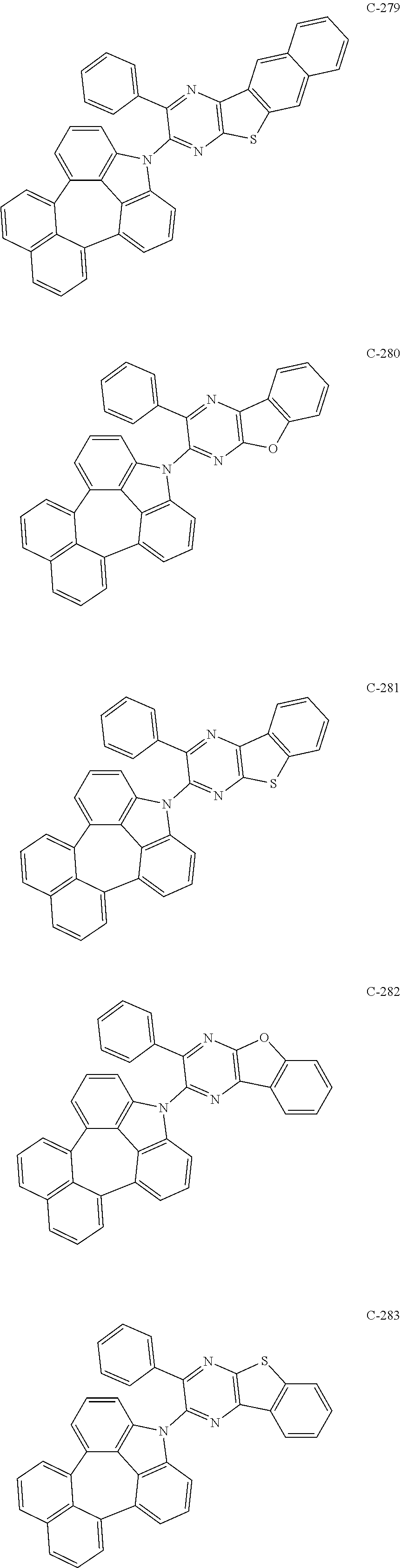 [SMILES: c1ccc(-c2nc3c(nc2-n2c4cccc5c4c4c(cccc42)-c2cccc4cccc-5c24)sc2cc4ccccc4cc23)cc1 ; c1ccc(-c2nc3c(nc2-n2c4cccc5c4c4c(cccc42)-c2cccc4cccc-5c24)oc2ccccc23)cc1 ; c1ccc(-c2nc3c(nc2-n2c4cccc5c4c4c(cccc42)-c2cccc4cccc-5c24)sc2ccccc23)cc1 ; c1ccc(-c2nc3oc4ccccc4c3nc2-n2c3cccc4c3c3c(cccc32)-c2cccc3cccc-4c23)cc1 ; c1ccc(-c2nc3sc4ccccc4c3nc2-n2c3cccc4c3c3c(cccc32)-c2cccc3cccc-4c23)cc1]

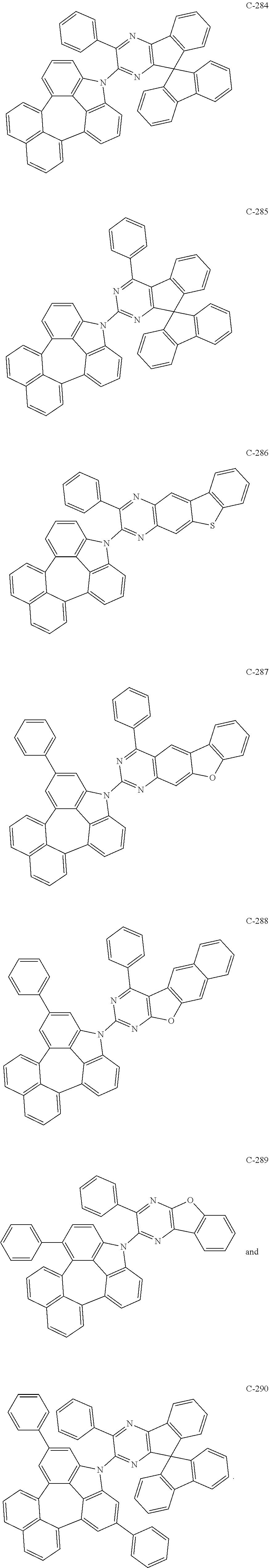

6. An organic electroluminescent material comprising the organic electroluminescent compound according to claim 1.

7. An organic electroluminescent device comprising the organic electroluminescent compound according to claim 1.

8. The organic electroluminescent device according to claim 7, wherein the organic electroluminescent compound is comprised in at least one of a light-emitting layer and an electron transport zone.
